(19) United States
(12) Patent Application Publication
(10) Pub. No.: US 2007/0149463 A1
(43) Pub. Date: Jun. 28, 2007

(54) Bifunctional macrolide heterocyclic compounds and methods of making and using the same

(76) Inventors: Adegboyega K. Oyelere, Marietta, GA (US); Jay J. Farmer, Lodi, NY (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/577,903

(22) PCT Filed: Oct. 29, 2004
(86) PCT No.: PCT/US04/36146

§ 371 Date: Jan. 17, 2007

Related U.S. Application Data
(60) Provisional application No. 60/515,909, filed on Oct. 30, 2003.

(51) Int. Cl. A61K 31/7052; C07H 17/08

(52) U.S. Cl. 514/28; 536/7.4

(57) ABSTRACT

The present invention relates generally to the field of anti-infective, anti-proliferative, anti-inflammatory, and prokinetic agents. More particularly, the invention relates to a family of bifunctional compounds useful as therapeutic agents. These compounds have both a macrolide ring and at least one heterocyclic moiety. The present invention further relates to processes for the preparation of such compounds, to intermediates useful in their preparation, to the use of the compounds as therapeutic agents, and to pharmaceuticals compositions containing them.

### RELATED APPLICATION

This application claims the benefit of and priority to U.S. patent application Ser. No. 60/515,909, filed Oct. 30, 2003, the disclosure of which is incorporated by reference herein.

### FIELD OF THE INVENTION

The present invention relates generally to the field of anti-infective, anti-proliferative, anti-inflammatory, and prokinetic agents, and more particularly, the invention relates to a family of bifunctional macrolide heterocyclic compounds useful as such agents.

### BACKGROUND

Since the discovery of penicillin in the 1920s and streptomycin in the 1940s, many new compounds have been discovered or specifically designed for use as antibiotic agents. It was once believed that infectious diseases could be completely controlled or eradicated with the use of such therapeutic agents. However, such beliefs have been challenged by the fact that strains of microorganisms resistant to currently effective therapeutic agents continue to evolve. Almost every antibiotic agent developed for clinical use has encountered problems with the emergence of resistant bacteria. For example, resistant strains of Gram-positive bacteria such as methicillin-resistant staphylocci, penicillin-resistant streptococci, and vancomycin-resistant enterococci have developed, and can cause serious and often time fatal results for patients infected with such resistant bacteria. Bacteria that are resistant to the macrolide antibiotics have developed. Also, Gram-negative strains of bacteria such as H. influenzae and M. catan-halis have been identified. See, e.g., F. D. Lowry, Antimicrobial resistance: the example of Staphylococcus aureus, J Clin. Invest., Vol. 111, No. 9, pp. 1265-1273 (2003); and Gold, H. S. and Moellering, R. C., Jr., Antimicrobial-drug resistance. N. Engl. J Med., vol. 335, 1445-53 (1996).

This problem of resistance is not limited to the area of anti-infective agents, because resistance has also been encountered with anti-proliferative agents used in cancer chemotherapy. Therefore, the need exists to develop new anti-infective and anti-proliferative agents that are both effective against resistant bacteria and strains of cells and against which bacteria and strains of cells are less likely to develop resistance.

Despite this problem of increasing antibiotic resistance, no new major classes of antibiotics have been developed for clinical use since the approval in the United States in 2000 of the oxazolidinone ring-containing antibiotic, N-[[(5S)-3-[3-fluoro-4-(4-morpholinyl)phenyl]-2-oxo-5-oxazolidinyl]methyl acetamide (see structure 1), which is known as linezolid and which is sold under the tradename Zyvox® (see compound A). See, R. C. Moellering, Jr., Linezolid: The First Oxazolidinone Antimicrobial, Annals of Internal Medicine, Vol. 138, No. 2, pp. 135-142 (2003).

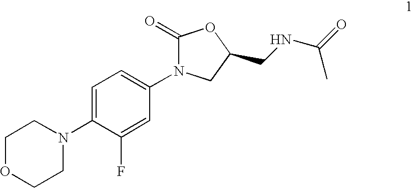

Linezolid was approved for use as an anti-bacterial agent active against Gram-positive organisms. However, linezolid-resistant strains of organisms are already being reported. See Tsiodras et al., Lancet, 2001, 358, 207; Gonzales et al., Lancet, 2001, 357, 1179; Zurenko et al., Proceedings Of The 39^{th} Annual Interscience Conference On Antibacterial Agents And Chemothierapy (ICAAC); San Francisco, Calif., USA, Sep. 26-29, 1999). However, investigators have been working to develop other effective linezolid derivatives. Research has indicated that the oxazolidinone ring could be important for linezolid's activity. The literature describes molecules having small groups substituted at the C-5 of the oxazolidinone ring, and early structure-activity relationships suggested that compounds with larger groups at the C-5 position were less active as anti-bacterial agents. As a consequence, investigators have been reluctant to place large substituents at the C-5 position of oxazolidinone rings in developing new anti-microbial agents.

Another class of antibiotics is the macrolides, which is so named for the 14- to 16-membered ring that is the major structural characteristic of this class of compounds. The first macrolide antibiotic to be developed was erythromycin, which was isolated from a soil sample from the Philippines in 1952. Even though erythromycin has been one of the most widely prescribed antibiotics, it has the disadvantages of relatively low bioavailability, gastrointestinal side effects, and a limited spectrum of activity. See Yong-Ji Wu, Highlights of Semi-synthetic Developments from Erythromycin A, Current Pharni. Design 6, pp. 181-223 (2000), and Yong-Ji Wu and Wei-uo Su, Recent Developments on Ketolides and Macrolides, Curr. Med. Chem., 8(14), pp.1727-1758 (2001).

In the search for new therapeutic agents, pharmaceutical researchers have tried combining or linking various portions of antibiotic molecules. However, this approach has met with limited success.

U.S. Pat. No. 5,693,791, to Truett, issued Dec. 2, 1997 describes an antibiotic of the formula:
A—L—B
wherein A and B are antibiotics selected from the group consisting of sulfonamides, penicillins, cephalosporins, quinolones, chloramphenicol, erytiromycin (i.e., a macrolide antibiotic), metronidzole, tetracyclines, and aminoglycosides. L is a linker formed from a difunctional linking agent.

PCT publication No. WO 99/63937, to Advanced Medicine, Inc., published Dec. 16, 1999, describes multi-inding compounds useful as antibiotics that are of the following formula:
(L)ₚ(X)_{q}
wherein L is selected from the group consisting of a macrolide antibiotic, an aminoglycoside, lincosamide, oxazolidinone, streptogramin, tetracycline, or another compound that binds to bacterial ribosomal RNA and/or to one or more proteins involved in ribosomal protein synthesis in the bacterium. In the formula, p is an integer from 2-10, q is an integer from 1-20, and X is a linker.

U.S. Pat. No. 6,034,069, to Or et al., issued Mar. 7, 2000 depicts a series of 3′-N-modified 6-O-substituted erythromycin ketolide derivatives such as shown below. R, R¹, and R² are selected from the group consisting of a variety of groups, including aryl-alkoxy-heteroaryl-alkylene. Rₚ is H or a hydroxy protecting group. W is absent or is O, NH, or NCH₃. R^{w} is H or an optionally substituted alkyl group.

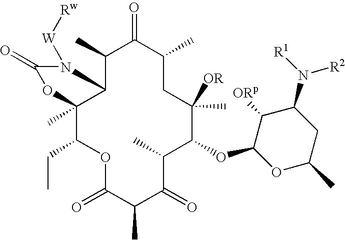

Notwithstanding the foregoing, there is an ongoing need for new anti-infective and anti-proliferative agents. In the present invention it has been found that new macrolide heterocyclic agents, including agents with relatively large substituents at the C5 position of an oxazolidinone ring and at similar positions in other heterocyclic rings can be prepared having good activity. Furthermore, because many anti-infective and anti-proliferative agents have utility as anti-inflammatory agents and also as prokinetic (gastrointestinal modulatory) agents, there is also an ongoing need for new compounds useful as anti-inflammatory and prokinetic agents. The present invention provides compounds which meet these needs.

### SUMMARY OF THE INVENTION

The present invention provides compounds useful as anti-infective agents and/or anti-proliferative agents, for example anti-microbial agents, antibacterial agents, antibiotic agents, anti-fungal agents, anti-parasitic agents, anti-viral agents, and chemotherapeutic agents. The present invention also provides compounds useful as anti-inflammatory agents, and/or prokinetic (i.e. gastrointestinal modulatory) agents. The present invention also provides pharmaceutically acceptable salts, ester, or prodrugs thereof.

The present invention provides compounds having both a macrolide ring and at least one heterocyclic moiety having the formula: 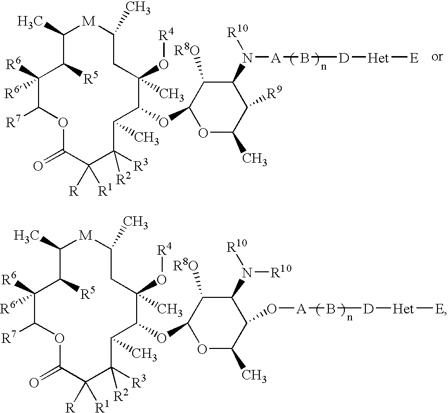
or a stereoisomer, or pharmaceutically acceptable salt, ester or prodrug thereof, wherein D-Het is selected from the group consisting of: 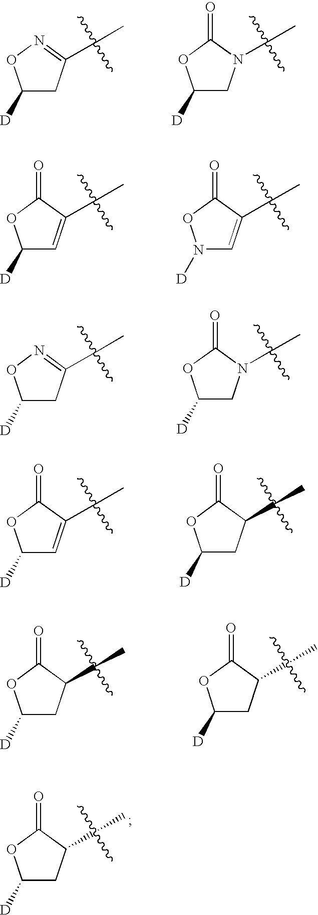
B is selected from the group consisting of:
   —C(O)NH—, —C(S)NH—, —NHC(O)—, —NHC(S)—, —S(O)₂NH—, —NHS(O)₂—, —OC(O)NH—, —OC(S)NH—, —NHC(O)NH—, —NHC(S)NH—, —NHC(O)O—, —NHC(S)O—, and —NR¹¹—;
n is 0 or 1, and the variables A, D, E, M, R, R¹R², R³, R⁴, R⁵, R⁶, R⁶′, R⁷, R⁸, R⁹, and R¹⁰ can be selected from the group consisting of the respective chemical moieties later defined in the detailed description.

In addition, the invention provides methods of synthesizing the foregoing compounds and useful chemical intermediates for synthesizing the foregoing compounds. Following synthesis, a therapeutically effective amount of one or more of the compounds can be formulated with a pharmaceutically acceptable carrier for administration to a mammal for use as an anti-cancer, anti-microbial, anti-biotic, anti-fungal, anti-parasitic or anti-viral agent, or to treat a proliferative disease, an inflammatory disease or a gastrointestinal motility disorder. Accordingly, the compounds or the formulations can be administered, for example, via oral, parenteral, or topical routes, to provide an effective amount of the compound to the mammal.

These and other aspects and embodiments of the invention can be more fully understood by reference to the following detailed description and claims.

### DETAILED DESCRIPTION OF THE INVENTION

The present invention provides a family of compounds that can be used as anti-proliferative agents and/or anti-infective agents. The compounds may be used without limitation, for example, as anti-cancer, anti-microbial, anti-bacterial, anti-fungal, anti-parasitic and/or anti-viral agents. Further, the present invention provides a family of compounds that can be used without limitation as anti-inflammatory agents, for example, for use in treating chronic inflammatory airway diseases, and/or as prokinetic agents, for example, for use in treating gastrointestinal motility disorders such as gastroesophageal reflux disease, gastroparesis (diabetic and post surgical), irritable bowel syndrome, and constipation.

The compounds described herein may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, and geometric isomeric forms of a structure are intended, unless the specific stereochemistry or isomeric form is specifically indicated. All processes used to prepare compounds of the present invention and intermediates made therein are considered to be part of the present invention. All tautomers of shown or described compounds are also considered to be part of the present invention.

1. Definitions

The term “substituted,” as used herein, means that any one or more hydrogens on the 25 designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N). The present invention, in general, does not cover groups such as N-halo, S(O)H, and SO₂H.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R³) occurs more than one time in any constituent or formula for a compound, its definition at each occurrence is independent of its definition at every other occurrence. Thus, for example, if a group is shown to be substituted with 0-2 R³ moieties, then the group may optionally be substituted with up to two R³ moieties and R³ at each occurrence is selected independently from the definition of R³. Also, combinations of substituents and/or variables are permissible, but only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent.

Combinations of substituents and/or variables are permissible, but only if such combinations result in stable compounds.

In cases wherein there are nitrogens in the compounds of the present invention, these can be converted to N-oxides by treatment with an oxidizing agent (e.g., MCPBA and/or hydrogen peroxides) to afford other compounds of the present invention. Thus, all shown and claimed nitrogens are considered to cover both the shown nitrogen and its N-oxide (N→O) derivative.

As used herein, “alkyl” is intended to include both branched and straight—Chain saturated aliphatic hydrocarbon groups having the specified number of carbon atoms. C₁₆ allryl is intended to include C₁, C₂, C₃, C₄, C₅, and C₆ alkyl groups. C₁₋₈ alkyl is intended to include C₁, C₂, C₃, C₄, C₅, C₆, C₇, and C₈ alkyl groups. Examples of alkyl include, but are not limited to, methyl, ethyl, n-propyl, i-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bonds that may occur in any stable point along the chain, such as ethenyl and propenyl. C₂₋₆ alkenyl is intended to include C₂, C₃, C₄, C5, and C₆ alkenyl groups. C₂₋₈ alkenyl is intended to include C₂, C₃, C₄, C₅, C₆, C₇, and C₈ alkenyl groups.

As used herein, “alkynyl” is intended to include hydrocarbon chains of either straight or branched configuration and one or more triple carbon-carbon bonds that may occur in any stable point along the chain, such as ethynyl and propynyl. C₂₋₆ alkynyl is intended to include C₂, C₃, C₄, Cs, and C₆ alkynyl groups. C₂₋₈ alkynyl is intended to include C₂, C₃, C₄, C₅, C₆, C₇, and C8 alkynyl groups.

As used herein, “cycloalkyl” is intended to include saturated ring groups, such as cyclopropyl, cyclobutyl, or cyclopentyl. C₃₋₈ cycloalkyl is intended to include C₃, C₄, C₅, C₆, C₇, and C₈ cycloalkyl groups.

As used herein, “halo” or “halogen” refers to fluoro, chloro, bromo, and iodo. “Counterion” is used to represent a small, negatively charged species such as chloride, bromide, hydroxide, acetate, and sulfate.

As used herein, “haloalkyl” is intended to include both branched and straight—Chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl.

As used herein, “alkoxy” refers to an alkyl group as defined above with the indicated number of carbon atoms attached through an oxygen bridge. C₁₋₆ alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “alkylthio” refers to an alkyl group as defined above with the indicated number of carbon atoms attached through an sulfur bridge. C₁₋₆ alkylthio, is intended to include C₁, C₂, C₃, C₄, C₅, and C₆ alkylthio groups. C₁₋₈ alkylthio, is intended to include C₁, C₂, C₃, C₄, C₅, C₆, C₇, and C₈ alkylthio groups.

As used herein, “carbocycle” or “carbocyclic ring” is intended to mean, unless otherwise specified, any stable 3, 4, 5, 6, 7, 8, 9, 10, 11, or 12-membered monocyclic, bicyclic or tricyclic ring, any of which may be saturated, unsaturated, or aromatic, recognizing that rings with certain numbers of members cannot be bicyclic or tricyclic, e.g., a 3-membered ring can only be a monocyclic ring. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl. As shown above, bridged rings are also included in the definition of carbocycle (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, the term “heterocycle” means, unless otherwise stated, a stable 3, 4, 5, 6, 7, 8, 9, 10, 11, or 12-membered monocyclic, bicyclic or tricyclic heterocyclic ring (recognizing that rings with certain numbers of members cannot be bicyclic or tricyclic, e.g., a 3-membered ring can only be a monocyclic ring), which is saturated, unsaturated, or aromatic, and consists of carbon atoms and one or more ring heteroatoms, e.g., 1 or 1-2 or 1-3 or 1-4 or 1-5 or 1-6 heteroatoms, independently selected from the group consisting of nitrogen, oxygen, and sulfur, and including any bicyclic group in which any of the above-defined heterocyclic rings is fused to a second ring (e.g., a benzene ring). The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “aromatic heterocycle” or “heteroaryl” is intended to mean a stable 5, 6, 7, 8, 9, 10, 11, or 12-membered monocyclic or bicyclic heterocyclic aromatic ring (recognizing that rings with certain numbers of members cannot be a bicyclic aromatic, e.g., a 5-membered ring can only be a monocyclic aromatic ring), which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoirnidazolyl, thiophenyl, triazinyl, 1,2,3-triazolyl, 1,2,4-triazolyl, 1,2,5-triazolyl, 1,3,4-triazolyl, and xanthenyl.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by malting acid or base salts thereof. Examples of pharmaceutically acceptable salts include, but are not limited to, mineral or organic acid salts of basic residues such as amines; ailcali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluene sulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, 1445.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent. It is preferred that the presently recited compounds do not contain a N-halo, S(O)₂H, or S(O)H group.

As used herein, “treating” or “treatment” means the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

As used herein, “mammal” refers to human and non-human patients.

As used herein, the term “therapeutically effective amount” refers to an amount of a compound, or a combination of compounds, of the present invention effective when administered alone or in combination as an anti-proliferative and/or anti-infective agent. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased anti-proliferative and/or anti-infective effect, or some other beneficial effect of the combination compared with the individual components.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

2. Compounds of the Invention

In one aspect, the invention provides compounds having the formula: 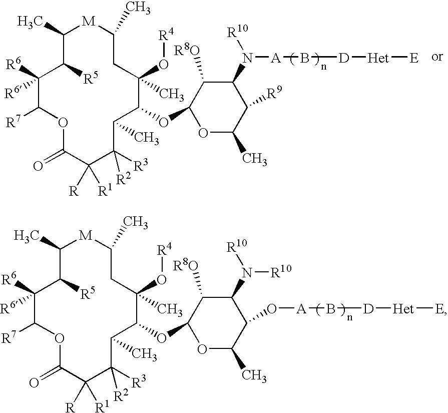
or pharmaceutically acceptable salt, ester or prodrug thereof,
wherein
D-Het is selected from the group consisting of: 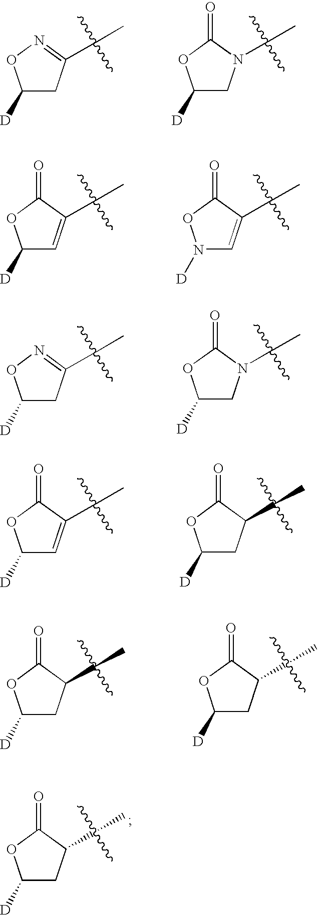
A is selected from the group consisting of:
   a) carbonyl, b) C₁₋₆ alkyl, c) C₂₋₆ alkenyl d) —C(O)—C₁₋₆ alkyl, and e) —C(O)—C₂₋₆ alkenyl,
   wherein
      i) 0-2 carbon atoms of the C₁₋₆ alkyl and C₂₋₆ alkenyl groups in any of b)-e) optionally are replaced by a moiety selected from the group consisting of O, S(O)p, and NR^{11,} and
      ii) any of b)-e) optionally is substituted with one or more R¹² groups;
B is selected from the group consisting of:
   a) —C(O)NH—, b) —C(S)NH—, c) —NHC(O)—, d) —NHC(S)—, e) —S(O)₂NH—, f) —NHS(O)₂—, g) —OC(O)NH—, h) —OC(S)NH—, i) —NHC(O)NH—, j) —NHC(S)NH—, k) —NHC(O)O—, l) —NHC(S)O—, and m) —NR¹¹—;
n is 0 or 1;
D is selected from the group consisting of:
   a) —CH₂—, b) —C(O)—, c) —C(S)—, d) —C(═NOR¹¹)—, e) —CH₂CH₂—, f) —OCH₂—, g) —SCH₂—, h) —S(O)CH₂—, i) —S(O)₂CH₂—, j) —NR¹¹CH₂—, k) —C(O)CH₂—, l) —C(S)CH₂—, and m) —C(═NOR¹¹)CH₂—;
E is selected from the group consisting of: 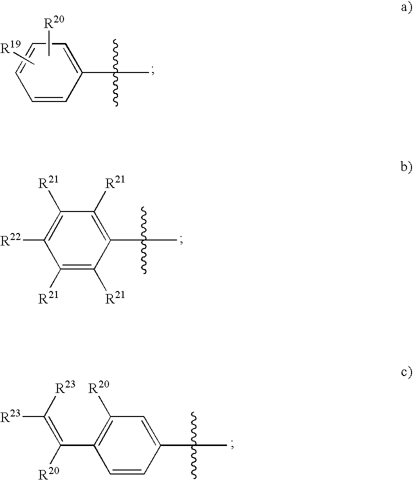
   d) 5-10 membered saturated, unsaturated, or aromatic heterocycle containing one or more heteroatoms selected from the group consisting of nitrogen, oxygen, and sur, and optionally substituted with one or more R¹² groups;
   e) C₅₋₁₀ saturated, unsaturated, or aromatic carbocycle, optionally substituted with one or more R¹² groups;
   f) C₁₋₈ alkyl,
   g) C₂₋₈ alkenyl,
   h) C₂₋₈ alkynyl,
   i) C₁₋₈ alkoxy,
   j) C₁₋₈ alkylthio,
   k) C₁₋₈ acyl,
   l) S(O)ᵣR¹¹; and
   m) hydrogen,
   wherein any of f)-k) optionally is substituted with
      i) one or more R¹² groups;
      ii) 5-6 membered saturated, unsaturated, or aromatic heterocycle containing one or more heteroatoms selected from the group consisting of nitrogen, oxygen, and sulfur, and optionally substituted with one or more R¹² groups; or
      iii) C₅₋₁₀ saturated, unsaturated, or aromatic carbocycle, optionally substituted with one or more R¹² groups;
M is selected from the group consisting of:
   a) —C(O)—, b) —C(═NOR¹¹)—, c) —CH(—OR¹¹)—, d) —NR¹¹—CH₂—, e) —CH₂—NR¹¹—, f) —CH(NR¹¹R¹¹)—, g) —C(═NNR¹¹R¹¹)—, h) —NR¹¹—C(O)—, i) —C(O)NR¹¹—, and j) —C(═NR¹¹)—;
R is selected from the group consisting of H and C₁₋₆ alkyl;
R¹¹is selected from the group consisting of:
   a) H, b) Cl, c) F, d) Br, e) I, f) —NR¹¹R¹¹ g) —NR¹¹C(O)R¹¹, h) —OR¹¹, i) —OC(O)R¹¹, j) —OC(O)OR¹¹, k) —OC(O)NR¹¹R¹¹, l) —O—C₁₋₆ alcyl-R¹², m) —OC(O)—C₁₋₆ alkyl-R¹², n) —OC(O)O—C₁₋₆ alkyl-R¹², o) —OC(O)NR¹¹-C₁₋₆ alkyl-R¹², p) C₁₋₆ alkyl, q) C₁₋₆ alkenyl, r) C₁₋₆ alkynyl,
      wherein any of l)-r) optionally is substituted with one or more R¹² groups;
R² is H;
R³ is selected from the group consisting of:
   a) H, b) —OR¹¹s, c) —O—C₁₋₆ alkyl-R¹², d) —OC(O)R¹¹, e) —OC(O)—C₁₋₆ alkyl-R¹², f) —OC(O)OR¹¹, g) —OC(O)O—C₁₋₆ alkyl-R¹², h) —OC(O)NR¹¹R¹¹, i) —OC(O)NR¹¹—C₁₋₆ alkyl-R¹², and 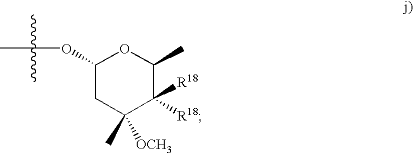
alternatively, R² and R³ taken together form a carbonyl group;
R⁴ is selected from the group consisting of:
   a) H, b) R¹¹, c) —C(O)R¹¹d) —C(O)OR¹¹e) —C(O)NR¹¹R¹¹, f) —C₁₋₆ alkyl-G—R¹¹, g) —C₂₋₆ alkenyl-G—R¹¹, and h) —C₂₋₆ alkynyl-G—R¹¹;
alternatively R³ and R⁴, taken together with the atoms to which they are bonded, form: 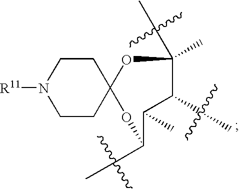
G is selected from the group consisting of:
   a) —C(O)—, b) —C(O)O—, c) —C(O)NR11—, d) —C(═NR¹¹)—, e) —C(═NR¹¹)O—, f) —C(═NR¹¹)NR¹¹—, g) —OC(O)—, h) —OC(O)O—, i) —OC(O)NR¹¹—, j) —NR¹¹C(O)—, k) —NR¹¹C(O)O—, l) —NR¹¹C(O)NR¹¹—, m) —NR¹¹C(═NR¹¹)NR¹¹—, and o) —S(O)^{p}—;
R⁵ is selected from the group consisting of:
   a) R¹¹, b) —OR¹¹, c) —NR¹¹R¹¹, d) —O—C₁₋₆ alkyl-R¹², e) —C(O)—R¹¹, f) —C(O)—C₁₋₆ alkyl-R¹², g) —OC(O)—R¹¹, h) —OC(O)—G₁₋₆ alkyl-R¹², i) —OC(O)O—R¹¹, j) —OC(O)O—C₁₋₆ alkyl-R¹², k) —OC(O)NR¹¹R¹¹, l) —OC(O)NR¹¹-C₁₋₆ alkyl-R¹², m) —C(O)—C₂₋₆ alkenyl-R¹², and n) —C(O)—C₂₋₆ alkynyl-R¹²;
alternatively, R⁴ and R⁵, taken together with the atoms to which they are bonded, form: 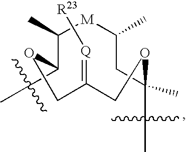
   wherein
      Q is CH or N, and
      R²³ is —OR¹¹, or R¹¹;
R⁶ is selected from the group consisting of:
   a) —OR¹¹, b) —C₁₋₆ alkoxy-R12, c) —C(O)R¹¹, d) —OC(O)R¹¹, e) —OC(O)OR¹¹, f) —OC(O)NR¹¹R¹¹, and g) —NR¹¹R¹¹;
alternatively, R⁵ and R⁶ taken together with the atoms to which they are attached form a 5-membered ring by attachment to each other through a linker selected from the group consisting of:
   a) —OC(R¹²)₂O—, b) —OC(O)O—, c) —OC(O)NR₁₁—, d) —NR¹¹C(O)O—, e) —OC(O)NOR¹¹—, f) —NOR¹¹—C(O)O—, g) —OC(O)NNR¹¹R¹¹—, h) —NNR¹¹R¹¹—C(O)O—, i) —OC(O)C(R¹²)₂—, j) —C(R¹²)₂C(O)O—, k) —OC(S)O—l) —OC(S)NR¹¹—, m) —NR¹¹C(S)O—, n) —OC(S)NOR¹¹—, o) —NOR¹¹—C(S)O—, p) —OC(S)NNR¹¹R¹¹—, q) —NNR¹¹R¹¹—C(S)O—, r) —OC(S)C(R¹²)₂—, and s) —C(R¹²)₂C(S)O—;
alternatively, M, R⁵, and R⁶ taken together with the atoms to which they are attached form: 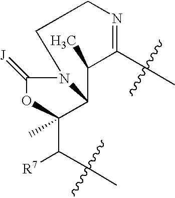
   wherein J is selected from the group consisting of O and NR¹¹;

R6′ is selected from the group consisting of
a) —H, b) —C₁₋₄ alkyl, c) C₂₋₄ alkenyl, which can be further substituted with C₁₋₂ alkyl or one or more halogens, d) C₂₋₄ alkynyl, which can be further substituted with C₁₋₂ alicyl or one or more halogens, e) aryl or heteroaryl, which can be further substituted with C₁₋₂ alkyl or one or more halogens, f) —C(O)H, g) —COOH, h) —CN, i) —COOR¹¹, j) —C(O)NR¹¹R¹¹, k) —C(O)R¹¹, and l) —C(O)SR¹¹, wherein b) is further substituted with one or more substituents selected from the group consisting of aa) —OR¹¹, bb) halogen, cc) —SR¹¹, dd) C₁₋₂ alkyl, which can be further substituted with halogen, hydroxyl, C₁₋₆ alkoxy, or amino, ee) —OR¹¹, ff) —SR¹¹, gg) —NR¹¹R¹¹, hh) —CN, ii) —NO₂, jj) —NC(O)R¹¹, kk) —COOR¹¹, ll) —N₃, mm) ═N—O—R¹¹, nn) ═NR¹¹, oo) ═N—NR¹¹R¹¹, pp) ═N—NH—C(O)R¹¹, and qq) ═N—NH—C(O)NR¹¹R¹¹;
alternatively R6 and R⁶′are talken together with the atom to which they are attached to form an epoxide, a carbonyl, an olefin, or a substituted olefin, or a C₃-C₇ carbocyclic, carbonate, or carbamate, wherein the nitrogen of said carbamate can be turther substituted with a C₁-C₆ alkyl;
R⁷ is selected from the group consisting of:
   a) C₁₋₆ alkyl, b) C₂₋₆ alkenyl, and c) C₂₋₆ alkynyl,
      wherein any of a)-c) optionally is substituted with one or more R¹² groups;
R⁸ is selected from the group consisting of H and —C(O)R¹¹;
R⁹ is selected from the group consisting of H, OH, and —OR¹¹;
R¹⁰ is selected from the group consisting of:
   a) H, b) R¹¹, c) —C₁₋₆ alkyl-G—R¹², d) —C₂₋₆ alkenyl-G—R¹², and e) —C₂₋₆ alkynyl-G—R¹²,
      wherein the C₁₋₆-alkyl, C₂₋₆ alkenyl, and C₂₋₆ alkynyl group in any of c)-e) optionally is substituted with one or more R¹² groups;
R¹¹, at each occurrence, independently is selected from the group consisting of:
   a) H, b) C₁₋₆ alkyl, c) C₂₋₆ alkenyl, d) C₂₋₆ alkynyl, e) C₆₋₁₀ saturated, unsaturated, or aromatic carbocycle, f) 3-12 membered saturated, unsaturated, or aromatic heterocycle containing one or more heteroatoms selected from the group consisting of nitrogen, oxygen, and sulfir, g) —C(O)—C₁₋₆ alkyl, h) —C(O)—C₂₋₆ alkenyl, i) —C(O)—C₂₋₆ alkynyl, j) —C(O)—C₆₋₁₀ saturated, unsaturate or aromatic carbocycle, kc) —C(O)—3-12 membered saturated, unsaturated, or aromatic heterocycle containing one or more heteroatoms selected from the group consisting of nitrogen, oxygen, sulfur, 1) —C(O)O—C₁₋₆ allyl, m) —C(O)O—C₂₋₆ alkenyl, n) —C(O)O—C₂₋₆ alkynyl, o) —C(O)O—C₆₋₁₀ saturated, unsaturated, or aromatic carbocycle, p) —C(O)O-3-12 membered saturated, unsaturated, or aromatic heterocycle containing one or more heteroatoms selected from the group consisting of nitrogen, oxygen, and sulfur, and q) —C(O)NR¹³R¹³,
      wherein any of b)-p) optionally is substituted with one or more R¹² groups,

alternatively, NR¹¹R¹¹ forms a 3-7 membered saturated, unsaturated or aromatic ring including the nitrogen atom to which the R¹¹ groups are bonded and optionally one or more moieties selected from the group consisting of: O, S(O)ₚ, and NR¹⁵;
R¹² is selected from the group consisting of:
   a) R¹⁴, b) C₁₋₈ alkyl, c) C₂₋₈ alkenyl, d) C₂₋₈ alkynyl, e) C₃₋₁₂ saturated, unsaturated, or aromatic carbocycle, f) 3-12 membered saturated, unsaturated, or aromatic heterocycle containing one or more heteroatoms selected from the group consisting of nitrogen, oxygen, and sulfur, and g) —NR¹⁵C(O)OR¹⁵,
      wherein any of b)-f) optionally is substituted with one or more R¹⁴ groups;
R¹³, at each occurrence, independently is selected from the group consisting of:
   a) H, b) C₁₋₆ allyl, c) C₂₋₆ alkenyl, d) C₂₋₆ alkynyl, e) C₃₋₁₀ saturated, unsaturated, or aromatic carbocycle, and f) 3-10 membered saturated, unsaturated, or aromatic heterocycle containing one or more heteroatoms selected from the group consisting of nitrogen, oxygen, and sulfur,
      wherein any of b)-f) optionally is substituted with one or more moieties selected from the group consisting of:
         carbonyl; formyl; F; Cl; Br; I; CN; NO₂; OR¹⁵; —S(O)ₚR¹⁵; —C(O)R^{15;} —C(O)OR¹⁵; —OC(O)R¹⁵; —C(O)NR¹⁵R¹⁵; —OC(O)NR¹⁵R¹⁵; —C(═NR¹⁵)R¹⁵; —C(R¹⁵)(R¹⁵)OR¹⁵; —C(R¹⁵)₂OC(O)R¹⁵; —C(R¹⁵)(OR¹⁵)(CH₂)ᵣNR¹⁵R¹⁵; —NR¹⁵R¹⁵; —NR¹⁵OR¹⁵; —NR¹⁵C(O)R¹⁵; —NR¹⁵C(O)OR¹⁵; —NR¹⁵C(O)NR¹⁵R¹⁵; —NR¹⁵S(O)ᵣR¹⁵; —C(OR¹⁵)(OR¹⁵)R¹⁵; —C(R¹⁵)₂NR¹⁵R¹⁵; ═NR¹⁵; —C(S)NR¹⁵R¹⁵; —NR¹⁵C(S)R¹⁵; —OC(S)NR¹⁵R¹⁵; —NR¹⁵C(S)OR¹⁵; —NR¹⁵C(S)NR¹⁵R¹⁵; —SC(O)R¹⁵; C₁₋₈ alkyl, C₂₋₈ alkenyl; C₂₋₈ alkynyl; C₁₋₈ alkoxy; C₁₋₈ alkylthio; C₁₋₈ acyl; saturated, unsaturated, or aromatic C₃₋₁₀ carbocycle; and saturated, unsaturated, or aromatic 3-10 membered heterocycle containing one or more heteroatoms selected from the group consisting of nitrogen, oxygen, and sulfur,

alternatively, NR¹³R¹³ forms a 3-10 membered saturated, unsaturated or aromatic ring including the nitrogen atom to which the R¹³ groups are attached and optionally one or more moieties selected from the group consisting of O, S(O)ₚ, NR¹⁵, and N;
alternatively, CR¹³R¹³ forms a carbonyl group;
R¹⁴, at each occurrence, is selected from the group consisting of:
   a) H, b) carbonyl, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣNR¹³(CR¹³R¹³)ₜR⁶, k) (CR¹³R¹³)ᵣOR¹⁶, l) (CR¹³R¹³)ᵣS(O)ₚ(CR¹³R¹³)ₜR¹⁶, m) (CR¹³R¹³)ᵣC(O)(CR¹³R¹³)ₜR¹⁶, n) (CR¹³R¹³)ᵣOC(O)(CR¹³R¹³)ₜR¹⁶, o) (CR¹³R¹³)ᵣSC(O)(CR¹³R¹³)ₜR¹⁶, p) (CR¹³R¹³)ᵣC(O)O(CR¹³R¹³)ₜR¹⁶, q) (CR¹³R¹³)ᵣNR¹³C(O)(CR¹³R¹³)ₜR¹⁶, r) (CR¹³R¹³)ᵣC(O)NR¹³(CR¹³R¹³)ₜR¹⁶, s) (CR¹³R¹³)ᵣC(═NR¹³)(CR¹³R¹³)ₜR¹⁶, t) (CR¹³R¹³)ᵣC(═NNR¹³R¹³)(CR¹³R¹³)ₜR¹⁶, u) (CR¹³R¹³)ᵣC(═NNR¹³C(O)R¹³)(CR¹³R¹³)ₜR¹⁶, v) (CR¹³R¹³)ᵣC(═NOR¹⁶)(CR¹³R¹³)ₜR¹⁶, w) (CR¹³R¹³)ᵣNR¹³C(O)O(CR¹³R¹³)ₜR¹⁶, x) (CR¹³R¹³)ᵣOC(O)NR¹³(CR¹³R¹³)ₜR¹⁶, y) (CR¹³R¹³)ᵣNR¹³C(O)NR¹³(CR¹³R¹³)ₜR¹⁶, z) (CR¹³R¹³)ᵣNR¹³S(O)ₚ(CR¹³R¹³)ₜR¹⁶, aa) (CR¹³R¹³)ᵣS(O)ₚNR¹³(CR¹³R¹³)ₜR¹⁶, bb) (CR¹³R¹³)ᵣNR¹³S(O)ₚNR¹³(CR¹³R¹³)ₜR¹⁶, cc) (CR¹³R¹³)ᵣNR¹³R¹³, dd) C₁₋₆ alkyl, ee) C₂₋₆ alkenyl, ff) C₂₋₆ alkynyl, gg) (CR¹³R¹³)ᵣ—C₃₋₁₀ saturated, unsaturated, or aromatic carbocycle, and hh) (CR¹³R¹³)ᵣ-3-10 membered saturated, unsaturated, or aromatic heterocycle containing one or more heteroatoms selected from the group consisting of nitrogen, oxygen, and sulfur,
      wherein any of dd)-hh) optionally is substituted with one or more R¹⁶ groups;
alternatively, two R¹⁴ groups may form —O(CH₂)ₛO—;
R¹⁵ is selected from the group consisting of:
   a) H, b) C₁₋₆ alkyl, c) C₂₋₆ alkenyl, d) C₂₋₆ alkynyl, e) C₃₋₁₀ saturated, unsaturated, or aromatic carbocycle, f) 3-10 membered saturated, unsaturated, or aromatic heterocycle containing one or more heteroatoms selected from the group consisting of nitrogen, oxygen, and sulfur, g) —C(O)—C₁₋₆ alkyl,
   h) —C(O)—C₁₋₆ alkenyl, g) —C(O)—C₁₋₆ alkynyl, i) —C(O)—C₃₋₁₀ saturated, unsaturated, or aromatic carbocycle, and j) —C(O)-3-10 membered saturated, unsaturated, or aromatic heterocycle containing one or more heteroatoms selected from the group consisting of nitrogen, oxygen, and sulfur,
      wherein any of b)-j) optionally is substituted with one or more moieties selected from the group consisting of H; F; Cl; Br; I; CN; NO₂; OH; NH₂; NH(C₁₋₆ alkyl); N(C₁₋₆ alkyl)₂; C₁₋₆ alkoxy; aryl; substituted aryl;

heteroaryl; substituted heteroaryl; and C₁₋₆ alkyl, optionally substituted with one or more moieties selected from the group consisting of aryl, substituted aryl, heteroaryl, substituted heteroaryl, F, Cl, Br, I, CN, NO₂, and OH;
R¹⁶, at each occurrence, independently is selected from the group consisting of:
   a) R¹⁷, b) C₁₆ alkyl, c) C₂₋₆ alkenyl, d) C₂₋₆ alkynyl, e) —C₃₋₁₀ saturated, unsaturated, or aromatic carbocycle, and f) -3-10 membered saturated, unsaturated, or aromatic heterocycle containing one or more heteroatoms selected from the group consisting of nitrogen, oxygen, and sulfur,
      wherein any of b)-f) optionally is substituted with one or more R¹⁷ groups;
R¹⁷, at each occurrence, independently is selected from the group consisting of:
   a) H, b) carbonyl, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣNR¹³R¹³, k) (CR¹³R¹³)ᵣOR¹¹, l) (CR¹³R¹³)ᵣS(O)ₚR¹³, m) (CR¹³R¹³)ᵣC(O)R¹³, n) (CR¹³R¹³)ᵣC(O)OR¹³, o) (CR¹³R¹³)ᵣOC(O)R^{13,} p) (CR¹³R¹³)ᵣNR¹³C(O)R¹³, q) (CR¹³R¹³)ᵣC(O)NR¹³R¹³, r) (CR¹³R¹³)ᵣC(═NR¹³)R¹³, s) (CR¹³R¹³)ᵣNR¹³C(O)NR¹³R¹³, t) (CR¹³R¹³)ᵣNR¹³S(O)ₚR¹³, u) (CR¹³R¹³)ᵣS(O)ₚNR¹³R¹³, v) (CR¹³R¹³)ᵣNR¹³S(O)ₚNR¹³R¹³, w) C₁₋₆alkyl, x) C₂₋₆ alkenyl, y) C₂₋₆ alkynyl, z) (CR¹³R¹³)ᵣ—C3-10 saturated, unsaturated, or aromatic carbocycle, and aa) (CR¹³R¹³)ᵣ-3-10 membered saturated, unsaturated, or aromatic heterocycle containing one or more heteroatoms selected from the group consisting of nitrogen, oxygen, and sulfur,
      wherein any of w)-aa) optionally is substituted with one or more moieties selected from the group consisting of R¹³; F; Cl; Br; I; CN; NO₂;
      —OR¹³; —NH₂; —NH(C₁₋₆ alkyl); —N(C₁₋₆ alkyl)₂; C₁₋₆ alcoxy; C₁₋₆ alkylthio; and C₁₋₆ acyl;
R¹⁸, at each occurrence, independently is selected from the group consisting of:
   a) H, b) OR¹⁵, c) —O—C₁₋₆ alkyl-OC(O)R¹⁵, d) —O—C₁₋₆ alkyl-OC(O)OR¹⁵, e) —O—C₁₋₆ alkyl-OC(O)NR¹⁵R¹⁵, f) —O—C₁₋₆ alkyl-C(O)NR¹⁵R¹⁵, g) —O—C₁₋₆ alkyl-NR¹⁵C(O)R¹⁵, h) —O—C₁₋₆ alkyl-NR¹⁵C(O)OR¹⁵, i) —0—C₁₋₆ alkyl-NR¹⁵C(O)NR¹⁵R¹⁵, j) —O—C₁₋₆ allyl-NR¹⁵C(═NH)NR15R¹⁵, k) —O—C₁₋₆ alkyl-S(O)ₚR¹⁵, l) —O—C₂₋₆ alkenyl-OC(O)R¹⁵, m) —O—C₂₋₆ alkenyl-OC(O)OR¹⁵, n) —O—C₂₋₆ alkenyl-OC(O)NR¹⁵R¹⁵, o) —O—C₂₋₆ alkenyl-C(O)NR¹⁵R¹⁵, p) —O—C₂₋₆ alkenyl-NR¹⁵C(O)R¹⁵, q) —O—C₂6 alkenyl-NR¹⁵C(O)OR¹⁵, r) —O—C₂₋₆ alkenyl-NR¹⁵C(O)NR¹⁵R¹⁵ , s) —O—C₂₋₆ alkenyl-NR¹⁵C(═NH)NR¹⁵R¹⁵, t) —O—C₂.₆ alkenyl-S(O)ₚR¹⁵, u) —O—C₂₋₆ alkynyl-OC(O)R¹⁵, v) —O—C₂₋₆ alkynyl-OC(O)OR¹⁵, W) —O—C₂₋₆ alkynyl-OC(O)NR¹⁵R¹⁵, x) —O—C₂₋₆ alkynyl-C(O)NR¹⁵R¹⁵, y) —O—C₂₋₆ alkynyl-NR¹⁵C(O)R¹⁵, z) —O—C₂₋₆ alkynyl-NR¹⁵C(O)OR¹⁵, aa) —O—C₂₋₆ alkynyl-NR¹⁵C(O)NR¹⁵R¹⁵, bb) —O—C₂₋₆ alkynyl-NR¹⁵C(═NH)NR¹⁵R¹⁵, cc) —O—C₂₋₆ alkynyl-S(O)ₚR¹⁵; and dd) —NR¹⁵R¹⁵;
alternatively, two R¹⁸ groups taken together form ═O, ═NOR¹⁵, or ═NNR¹⁵R¹⁵;
R¹⁹ is R¹²;
R²⁰ is selected from the group consisting of:
   a) R¹³, b) F, c) cl, d) Br, e) I, f) CN. g) NO₂, and h) —OR¹¹;
alternatively, R¹⁹ and R²⁰ taken together are —O(CH₂)ᵣO—;
R²¹, at each occurrence, independently is selected from the group consisting of:
   a) H, b) F, c) Cl, d) Br, e) I, f) CN, g) —OR¹¹, h) NO₂, i) —NR¹¹R¹¹, j) C₁₋₆ alkyl, k) C₁₋₆ acyl, and 1) C₁₋₆ alkoxy;
R²² is selected from the group consisting of:
   a) C₁₋₆ alkyl, b) C₂₋₆ alkenyl, c) C₂₋₆ alkynyl, d) C₁₋₆ acyl, e) C₁₋₆ alkcoxy, f) C₁₋₆ alkylthio, g) saturated, unsaturated, or aromatic C₅₋₁₀ carbocycle, h) saturated, unsaturated, or aromatic 5-10 membered heterocycle containing one or more heteroatoms selected from the group consisting of nitrogen, oxygen, and sulfur, i) —O—C₁₋₆ alkyl-saturated, unsaturated, or aromatic 5-10 membered heterocycle containing one or more heteroatoms selected from the group consisting of nitrogen, oxygen, and sulfur, j) —NR¹¹—C₁₋₆ alkyl-saturated, unsaturated, or aromatic 5-10 membered heterocycle containing one or more heteroatoms selected from the group consisting of nitrogen, oxygen, and sulfur, k) saturated, unsaturated, or aromatic 10-membered bicyclic ring system optionally containing one or more heteroatoms selected from the group consisting of nitrogen, oxygen, and sulfur, 1) saturated, unsaturated, or aromatic 13-membered tricyclic ring system optionally containing one or more heteroatoms selected from the group consisting of nitrogen, oxygen, and sulfur, m) —OR₁₁, n) —NR¹¹R¹¹, o) S(O)ᵣR¹¹, and p) R²¹,
      wherein any of a)-l) optionally is substituted with one or more R¹² groups;
alternatively, R²² and one R²¹ group, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;
R²³ at each occurrence, independently is selected from the group consisting of:
   a) hydrogen; b) an electron-withdrawing group; c) aryl; d) substituted aryl; e) heteroaryl; f) substituted heteroaryl; and g) C₁₋₆ allyl, optionally substituted with one or more R¹² groups;
alternatively, any R²³ and any R²⁰, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;
p, at each occurrence, is selected from the group consisting of 0, 1, and 2;
r, at each occurrence, is selected from the group consisting of 0, 1, and 2;
s, at each occurrence, is selected from the group consisting of 1, 2, 3, or 4;
t, at each occurrence, is selected from the group consisting of 0, 1, or 2;
u, at each occurrence, is selected from the group consisting of 1, 2, 3, 4, or 5; and,
v, at each occurrence, is selected from the group consisting of 0, 1, 2, or 3.

Embodiments of the foregoing compounds include those compounds having the formula: 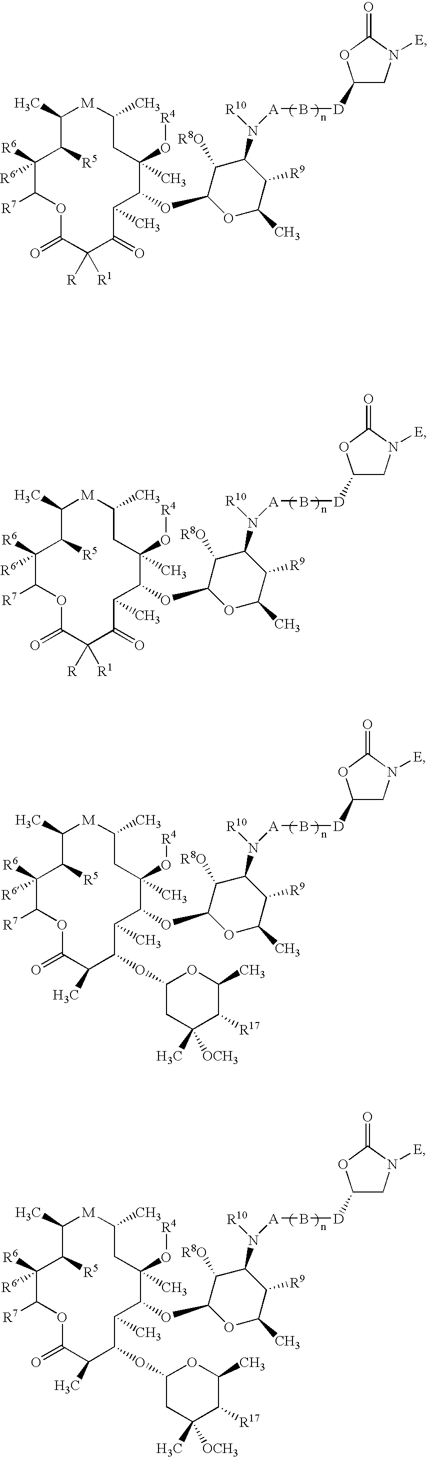 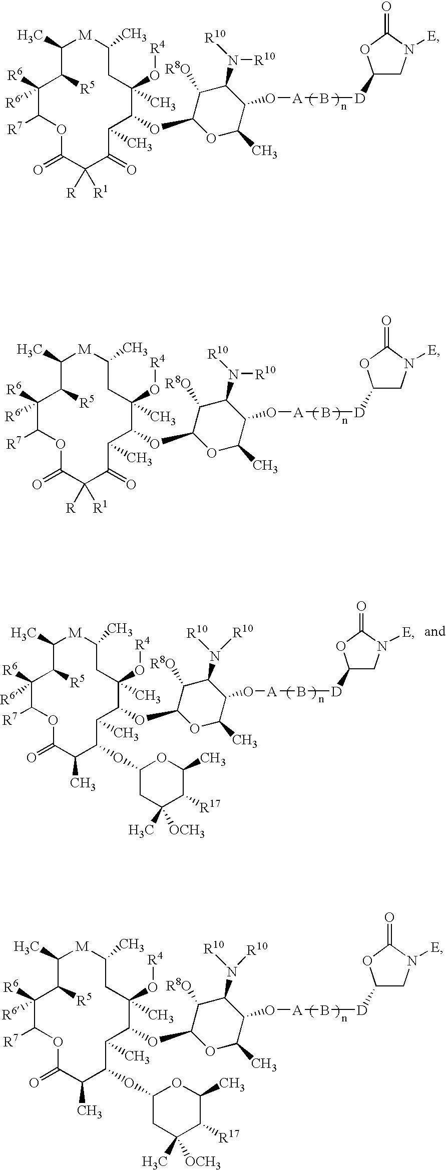
or a pharmaceutically acceptable salt, ester, or prodrug thereof, wherein A, B, n, D, E, R, R¹, R⁴, R⁵, R⁶, R⁶′, R7, R⁸, R⁹, and R¹⁰ are as defined hereinabove.

Other embodirnents of the foregoing compounds include those compounds having the formula: 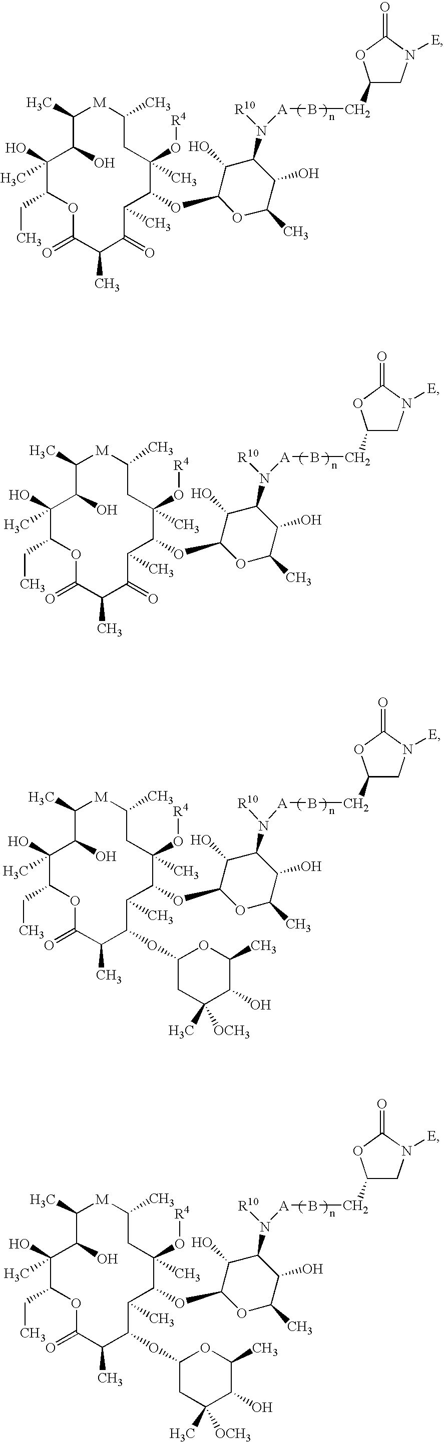 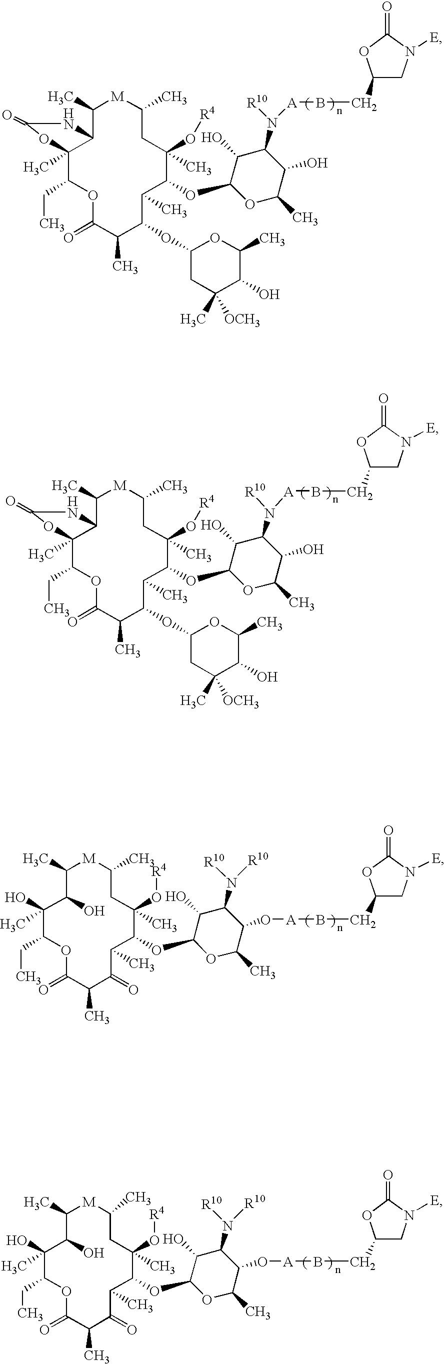 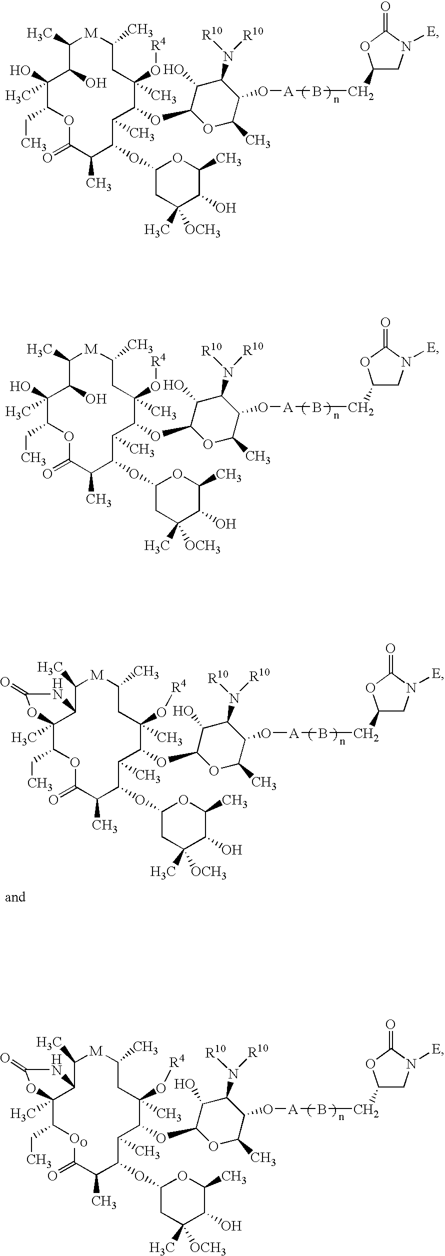
or a pharmaceutically acceptable salt, ester, or prodrug therefore, wherein A, B, n, E, R⁴, and R¹⁰ are as defined hereinabove.

Still other embodiments of the foregoing compounds include those compounds having the formula selected from the group consisting of: 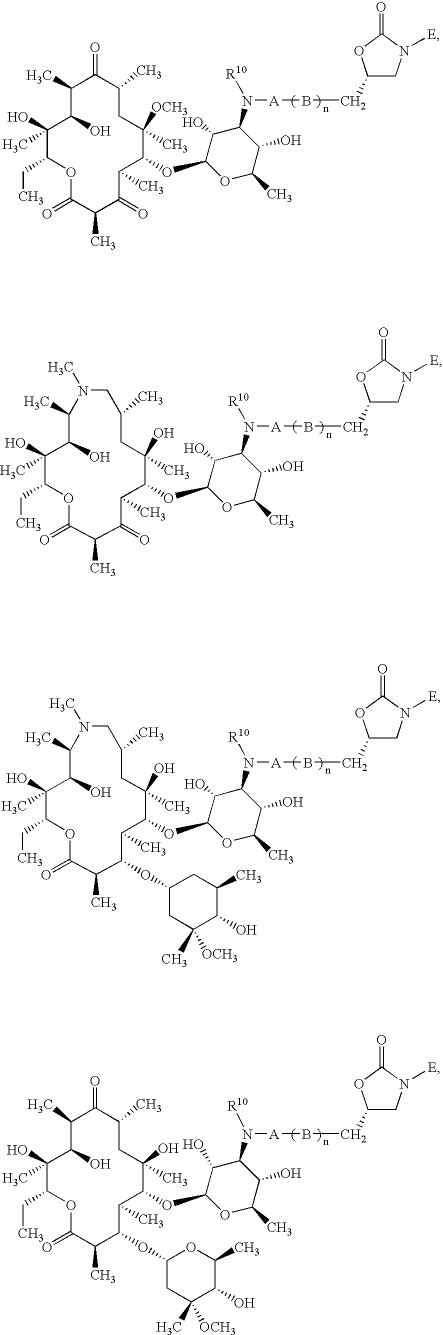 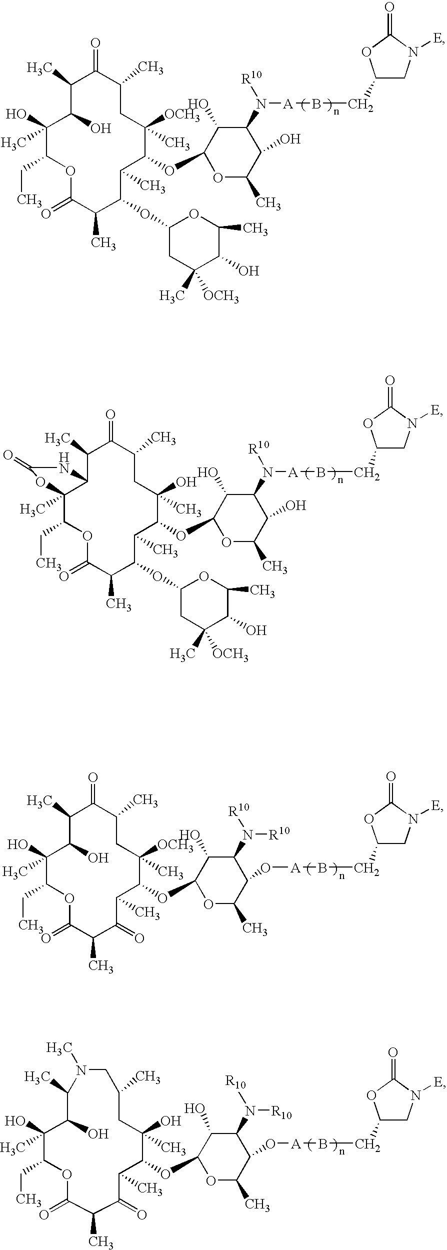 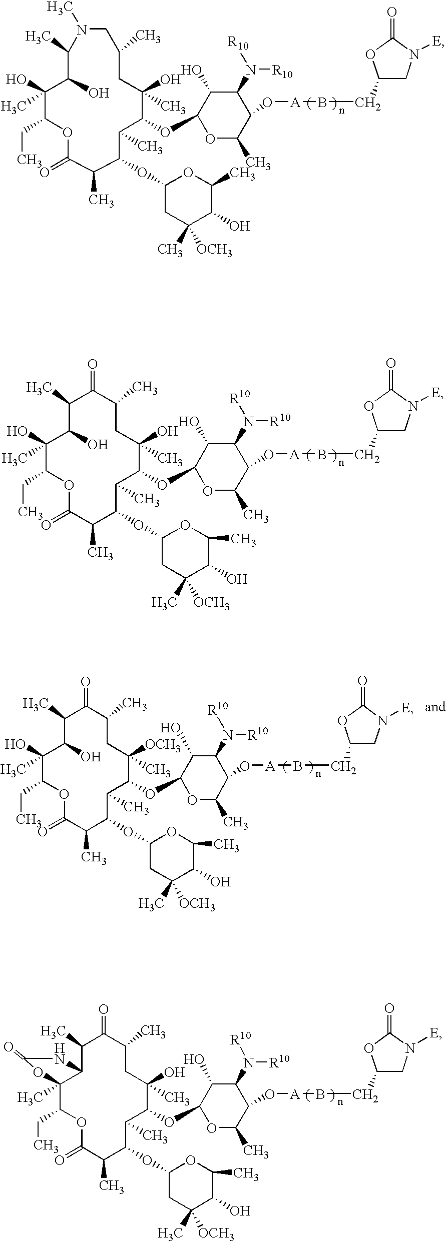
or a pharmaceutically acceptable salt, ester, or prodrug thereof, wherein A, B, n, E, and R¹⁰ are as defined hereinabove.

In certain embodiments of the invention, n is 1. In other embodiments, A-(B)ₙ-D has the formula A—C(O)NH—D, A—SO₂NH—D, or A—C(S)NH—D.

Other embodiments of the invention include compounds having the formula: 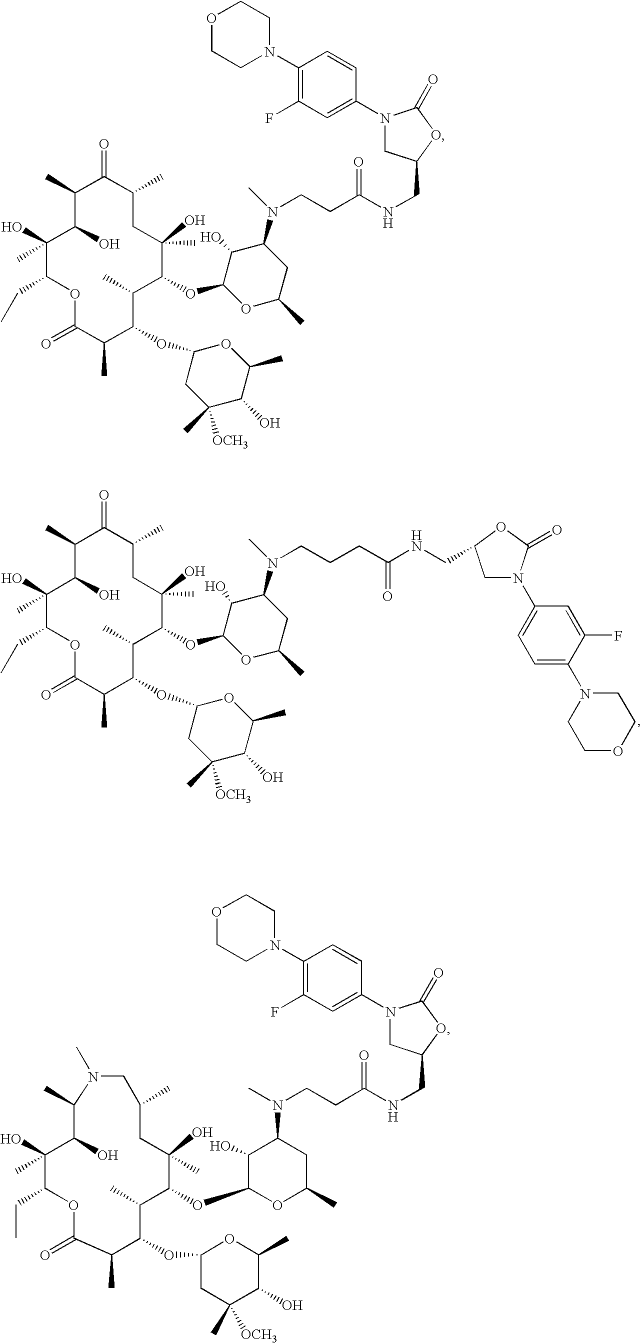 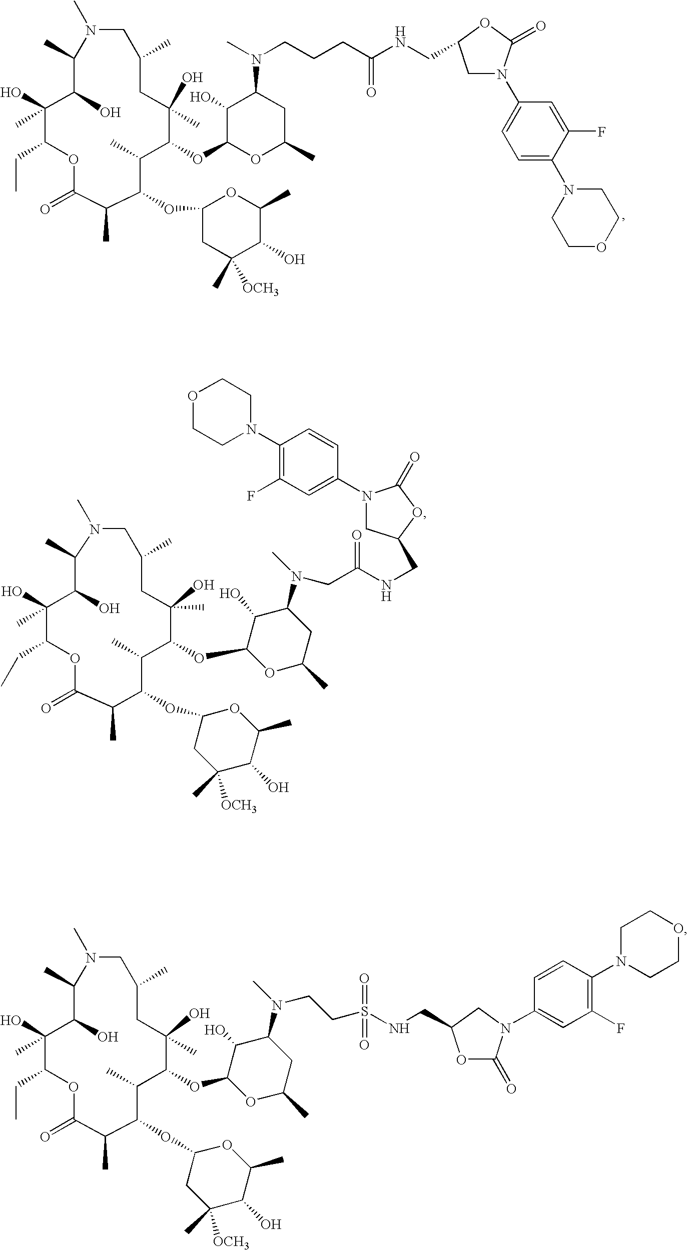 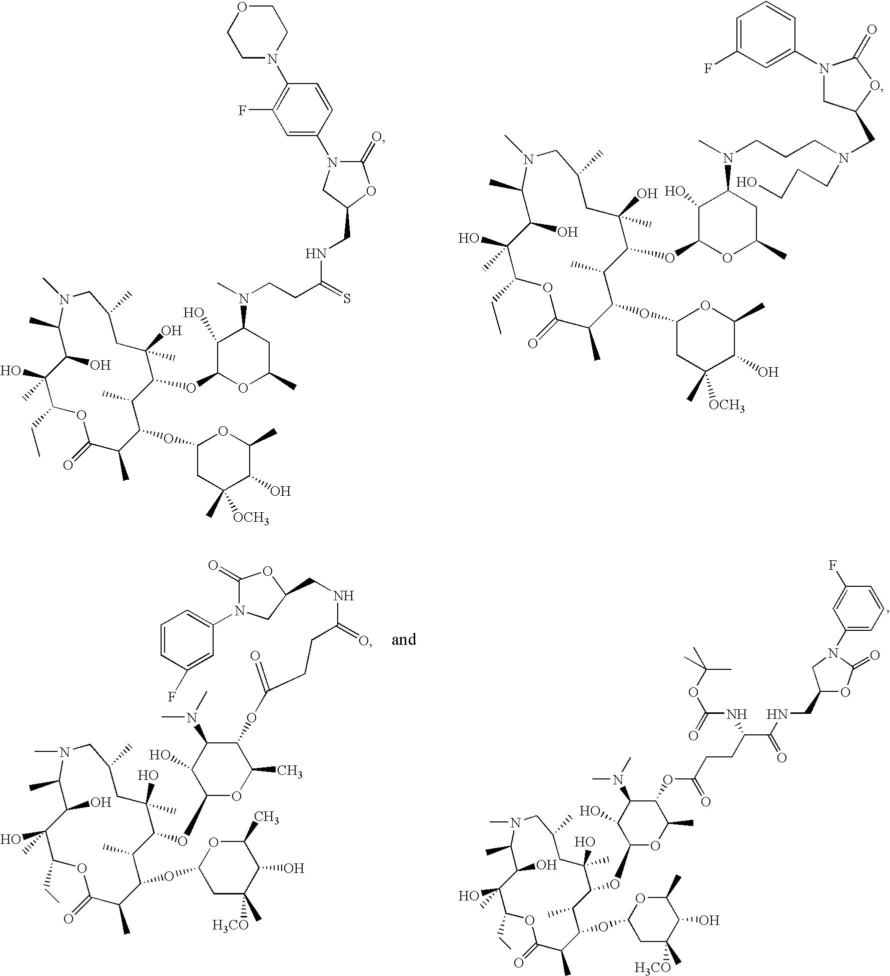
or a pharmaceutically acceptable salt, ester, or prodrug thereof.

In another aspect, the invention provides a pharmaceutical composition comprising a therapeutically effective amount of one or more of the foregoing compounds and a pharmaceutically acceptable carrier. In yet another aspect, the invention provides a method for treating a microbial infection, a fungal infection, a parasitic disease, a proliferative disease, a viral infection, an inflammatory disease, or a gastrointestinal motility disorder in a mammal by administering effective amounts of the compounds of the invention or pharmaceutical compositions of the invention. In embodiments of this aspect, the compounds are administered orally, parentally, or topically. In still another aspect, the invention provides methods for synthesizing any one of the foregoing compounds. In another aspect, the invention provides a medical device, for example, a medical stent, which contains or is coated with one or more of the foregoing compounds.

The invention further provides a family of hybrid antibiotics comprising at least a portion of an heterocyclic side—chain linked via a non-aromatic linker to a macrolide. Exemplary macrolides, linkers, and heterocyclic side—chains useful in the synthesis of the antibiotics include, but are not limited to, the chemical moieties shown below.
Macrolides 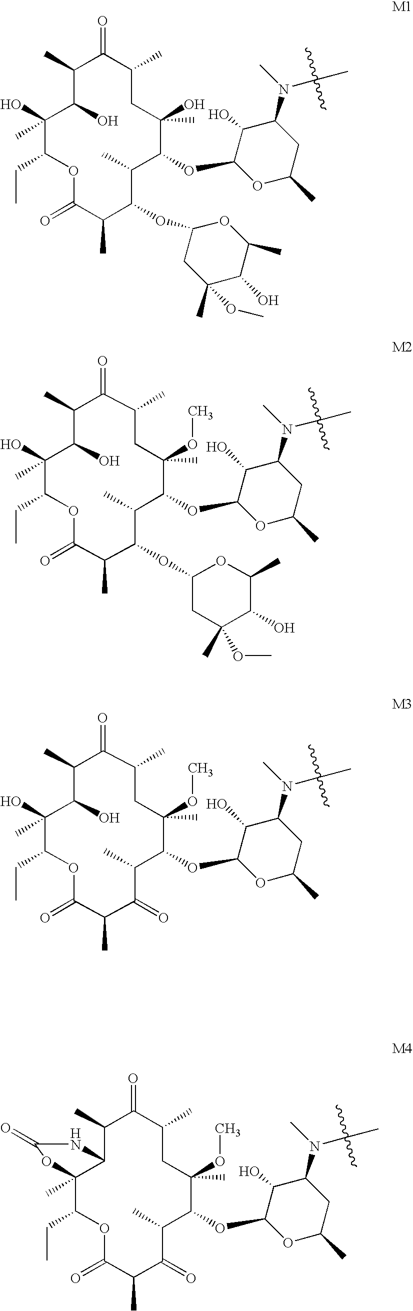 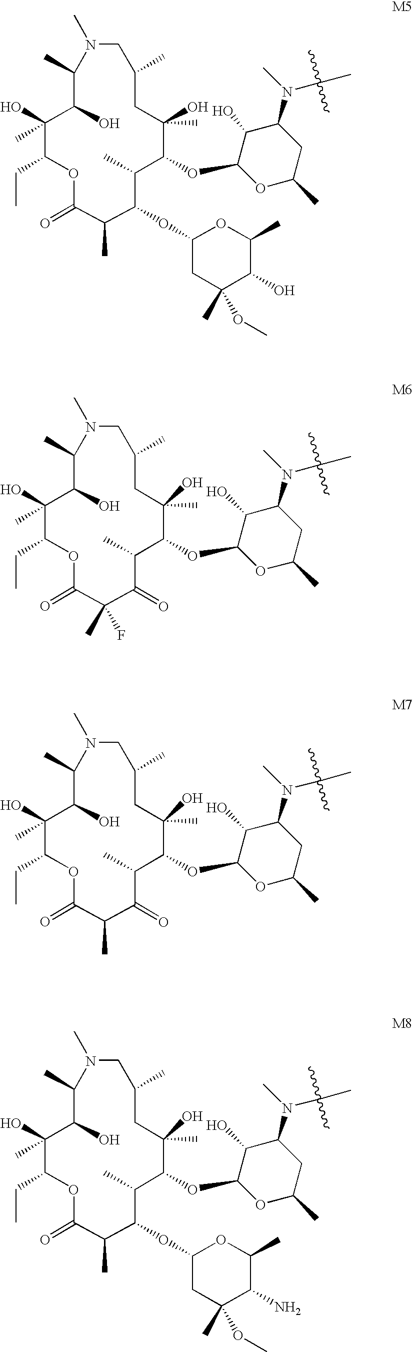 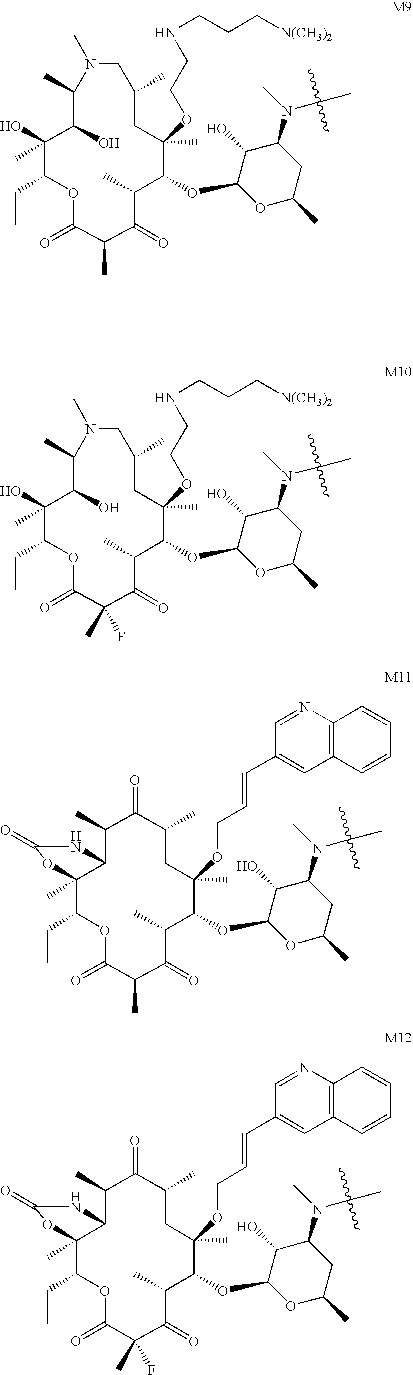 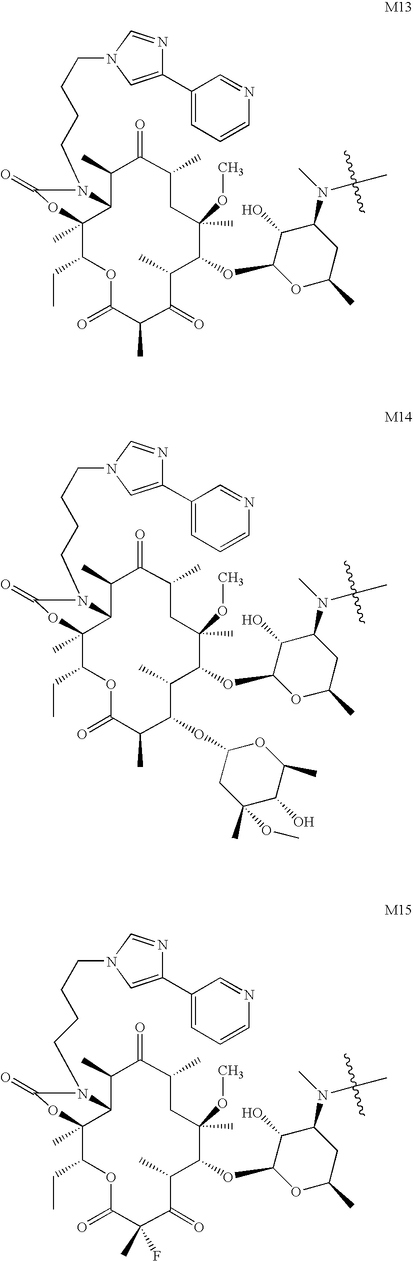 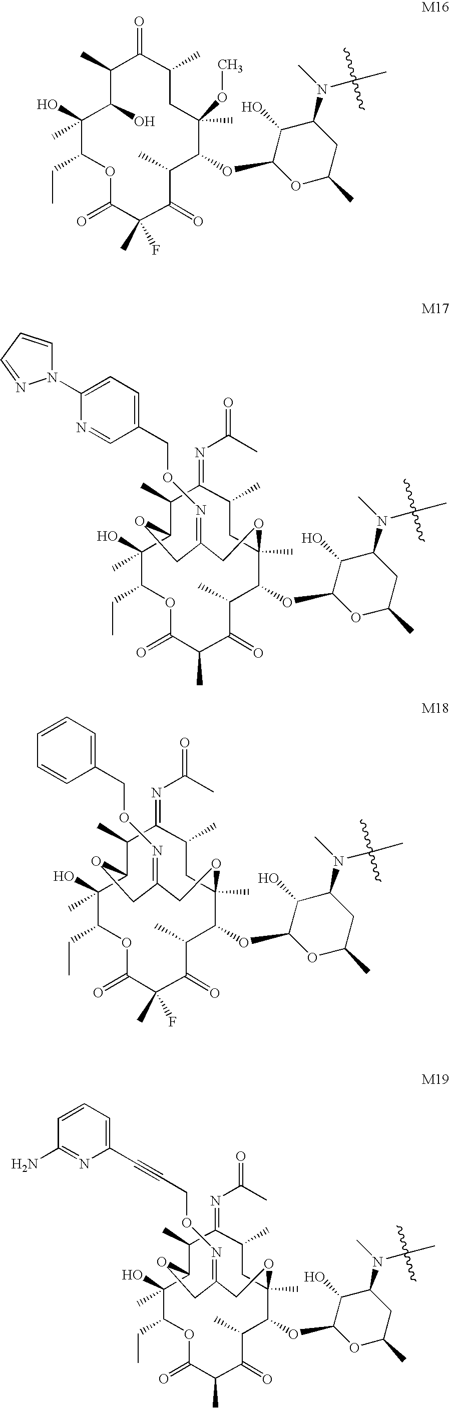 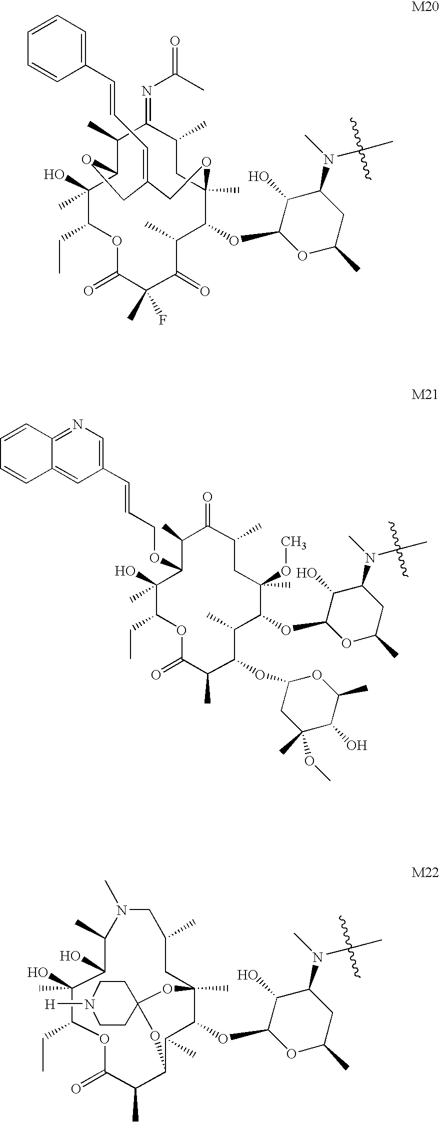
Linkers

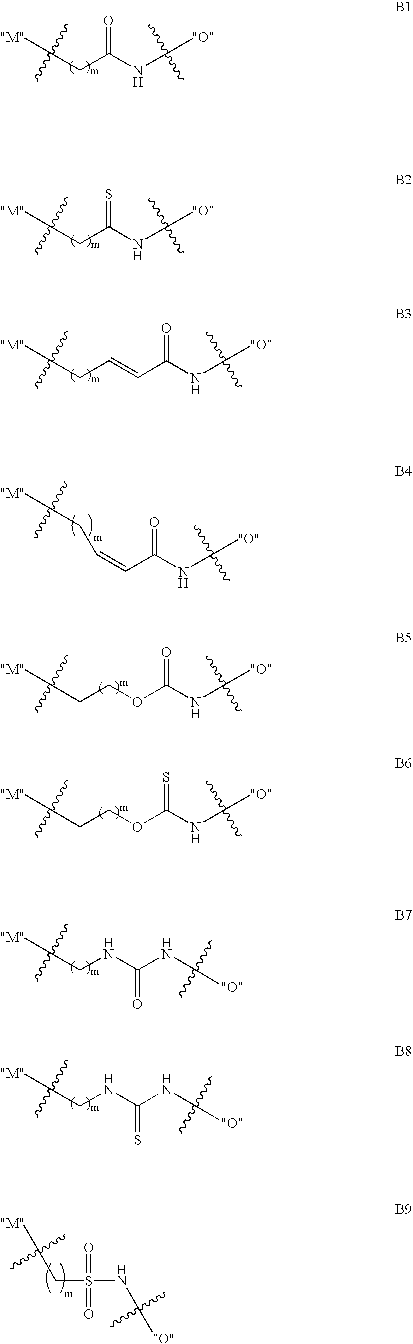

For the above linker groups, it should be understood that “M” and “O” are included to depict the orientation of the linker group with respect to the other structures that define the compounds of the invention. More specifically, “M” denotes the portion of the compound that includes the macrolide, and “O” denotes the portion of the compound that includes the heterocyclic side-chain.
Heterocyclic Side-Chains

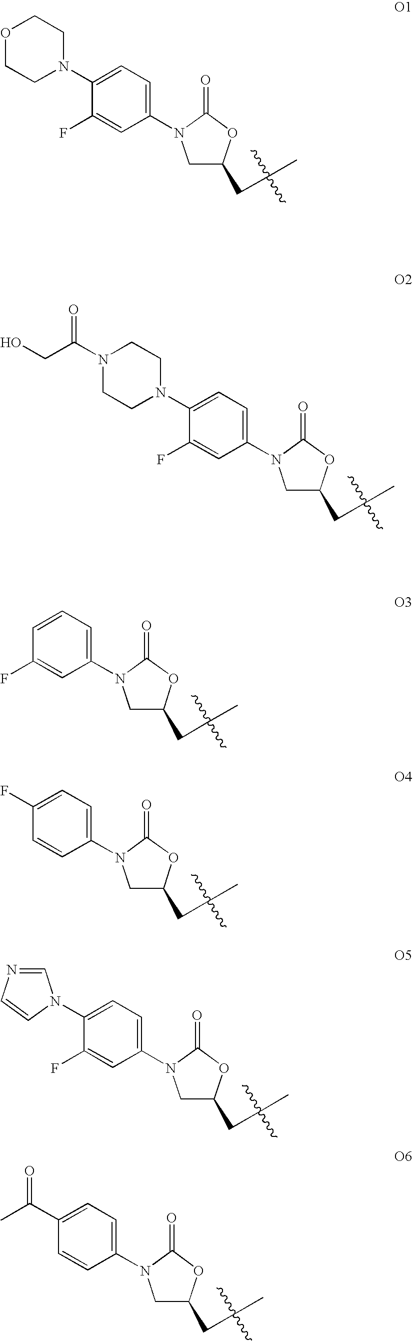

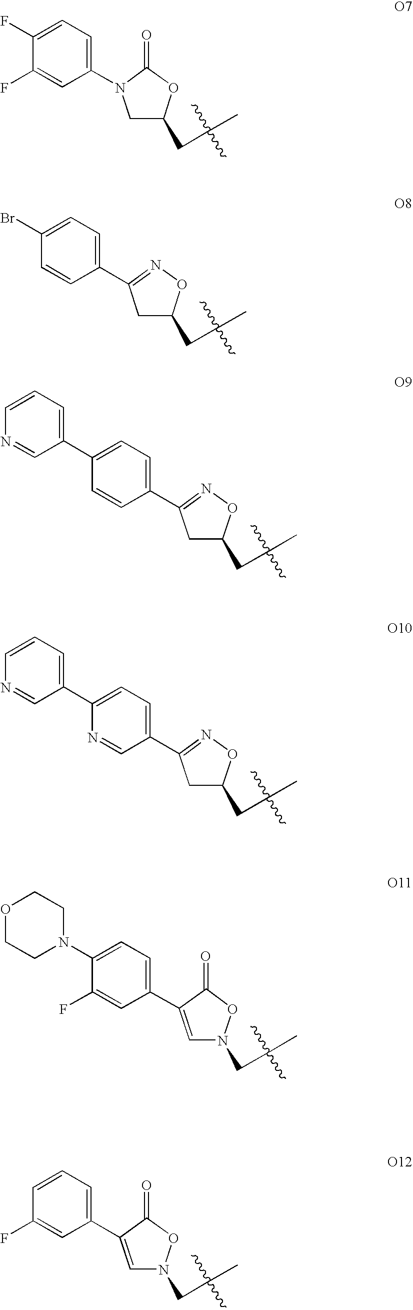

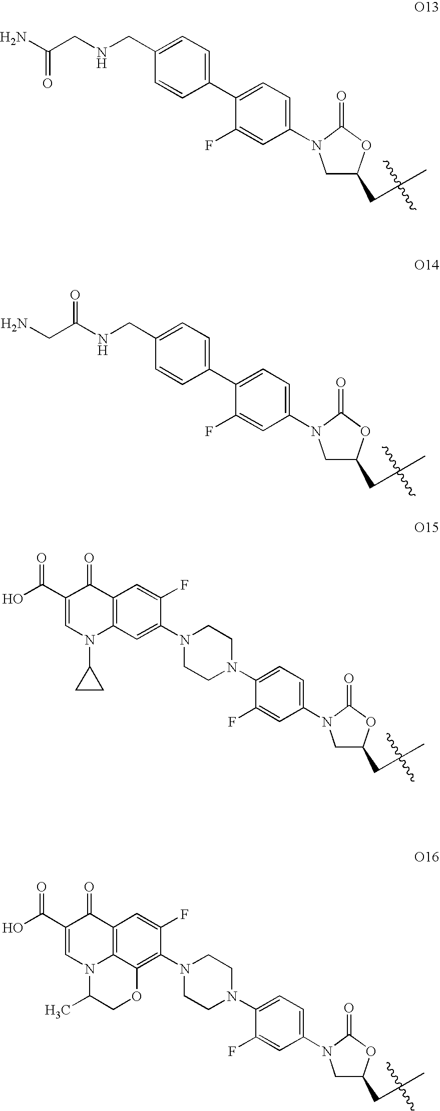

An exemplary scheme showing the linkage of a macrolide to a heterocyclic side-chain via a linker is set below, where m can be 1, 2, 3, or 4:

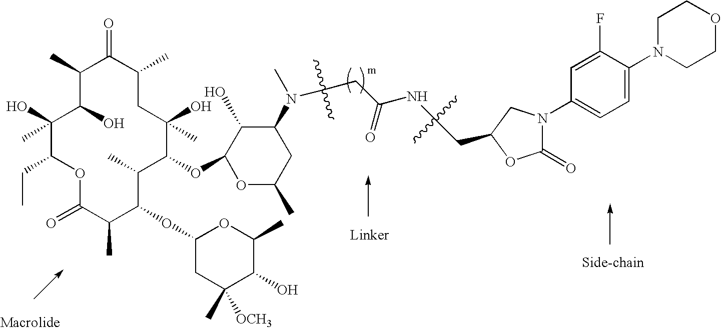

The above exemplary scheme can be represented as follows:
M—(CH₂)^{m}—B—O
wherein M is a macrolide selected from the group consisting of M1 through M22, as shown above, B is a linker selected from the group consisting of B1 through B9 as shown above, O is a heterocyclic side chain selected from the group consisting of O1 through O16 as shown above, and m is an integer from 1-4.

The various macrolides can be linked via the linkers to the heterocyclic side-chain using conventional chemistries known in the art, such as those discussed above. By using the various combinations of chemical moieties provided, the skilled artisan may synthesize one or more of the exemplary compounds of the present invention. A non-limiting example of a compound that one can make based on these exemplary moieties would be wherein the macrolide moiety is selected from M1, the linker moiety is selected from B1 with the variable length chain portion being selected from m=1, and the heterocyclic side chain moiety is selected from O1. One skilled in the art would recognize that these illustrated moieties alone can be combined to describe 12,672 unique compounds, i.e. [22 macrolides]×[9 linkers]×[4 chain lengths for each linker]×[16 heterocyclic side chains]=12, 672.

3. Synthesis of the Compounds of the Invention

In another aspect, the invention provides methods for making the compounds of the invention. The following schemes depict some exemplary chemistries available for synthesizing the compounds of the invention. It will be appreciated, however, that the desired compounds may be synthesized using other alternative chemistries known in the art.

The dimethyl amino group of the desosamine sugar of macrolide antibiotics can be monodemethylated to produce the corresponding secondary amine (U.S. Pat. No. 3,725,385, Flynn et al. (1954) J. AM. CHEM. SOC. 76: 3121; Ku et al. (1997) BIOORG. MED. CHEM. LETT. 7: 1203; Stenmark et al. (2000) J. ORG. CHEM. 65: 3875). For example, desosamine derivative 1 is available from the degradation of erythromycin. The same chemistry can be employed to produce amine 2 from azithromycin.

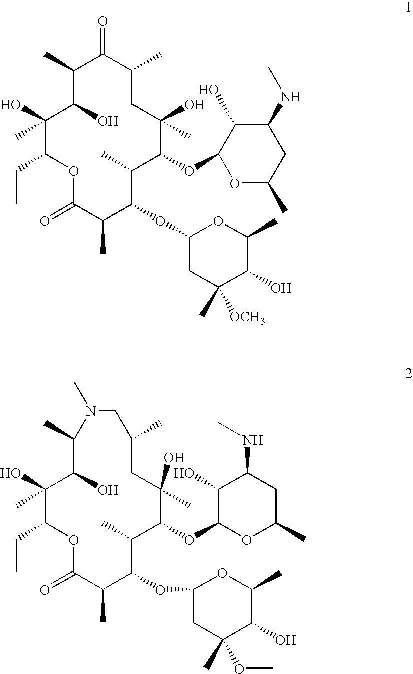

Scheme 1 illustrates how amines 1 and 2 can be alkylated with appropriate electrophiles to produce compounds of the present invention that employ linker group L1. Known amine 3 (for a synthesis see: Brickner et al. (1996) J. MED. CHEM. 39: 673) was acylated with the appropriate bromoacids to afford bromides 4 and 5. Bromides 4 and 5 were then used to allkylate amine 1 to afford targets 6 and 7, and amine 2 to yield 8 and 9. Compounds of type 6-9 can be converted to thioamide targets of linker type L2 by their reaction with Lawesson's reagent or P₂S₅, or many other reagent combinations known to those skilled in the art. It will be understood that a variety of amines similar to 3 could be employed in the chemistry of Scheme 1 (and all subsequent schemes) to provide compounds of the present invention. It is intended that these are within the scope of the present invention.

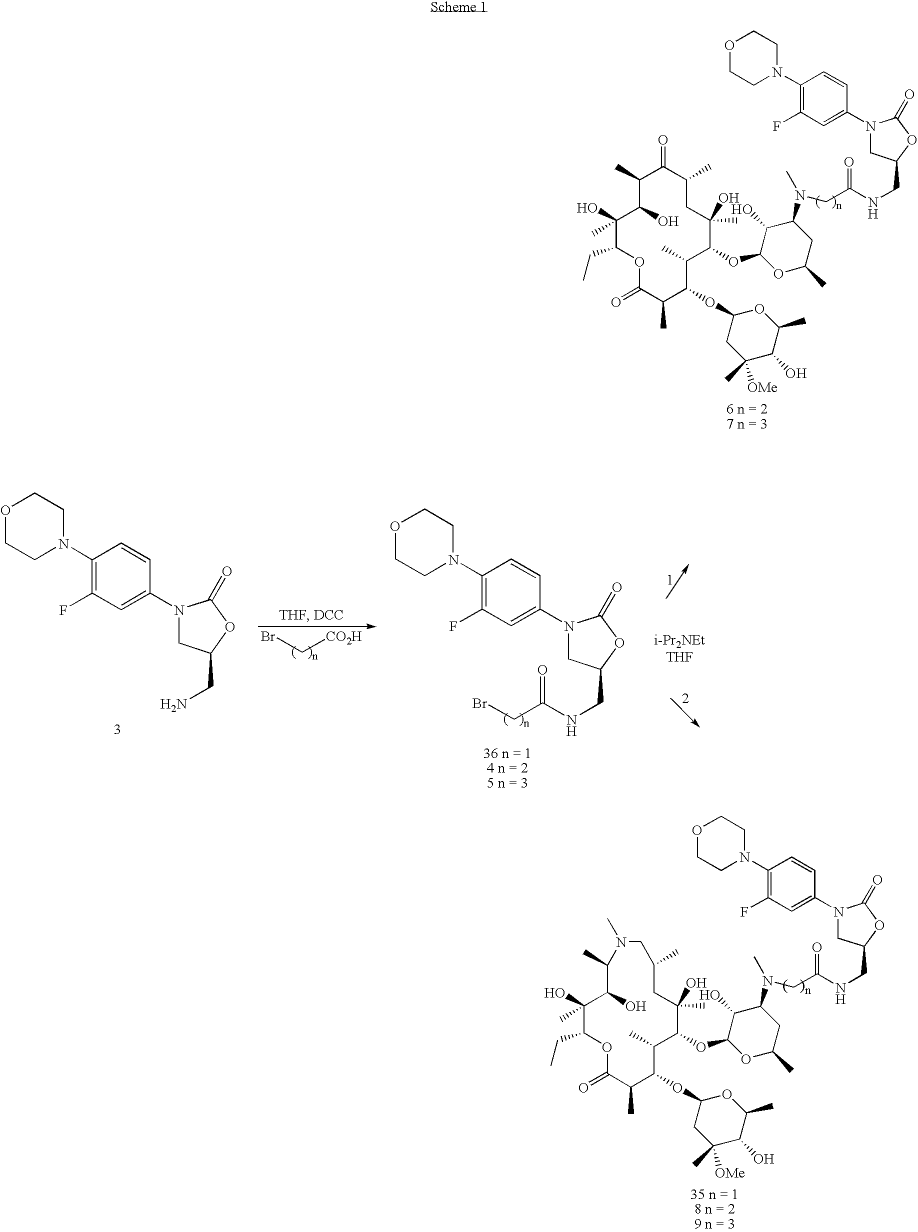

Compounds containing linker group L3 can be made for example by the chemistry shown in Scheme 2. Oxazolidinone amines of type 3 be acylated with commercially available bromoacid 10, or the known bromoacid 11 (Bellassoued et al. (1985) TETRAHEDRON 41: 1299), to afford intermediates of type 12 and 13. Alkylation of amines of type 1 and 2 (as non-limiting examples) will provide derivatives of type 14-17.

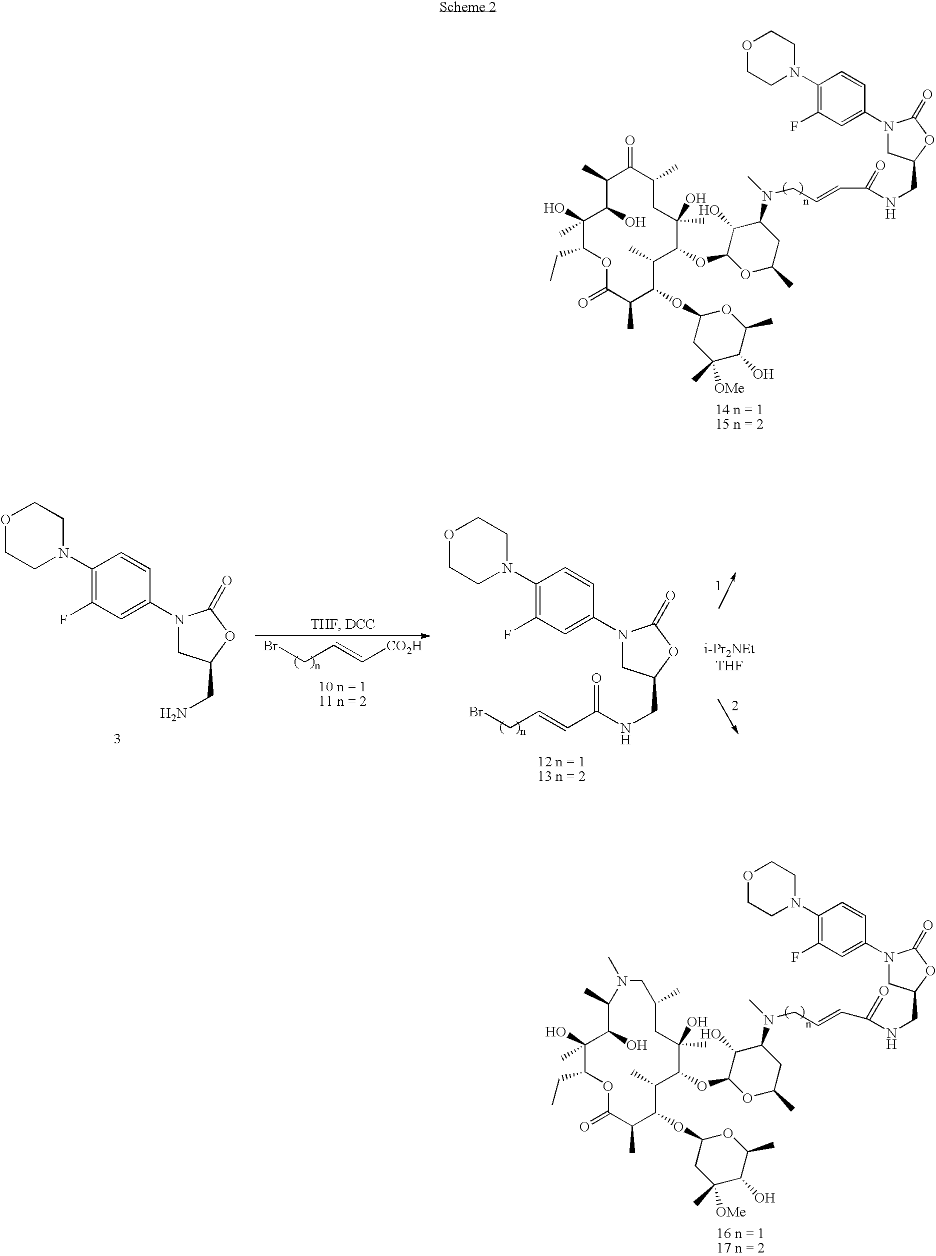

Scheme 3 depicts how cis olefin of targets such as 22-25 (with linker group L4) can be made from acetylenic amides of type 20 and 21. Amines of type 3 can be acylated with the known acetylenic acids 18 (Tsou et al. (2001) J. MED. CHEM. 44: 2719) and 19 (Carey et al. (2001) J. ORG. CHEM. 66: 2526) to afford alkynes 20 and 21 respectively. Hydrogenation of 20 and 21 with hydrogen and Lindlar's catalyst (or other conditions such as diimide reduction) will provide cis olefin intermediates that can be desilylated with tetrabutylammonium fluoride (or acidic conditions such as a mixture of acetic acid in tetrahydrofuran and water) to afford the corresponding alcohols. These alcohols can be converted to tosylates (or other electrophilic species such as a halides) using chemistry well known in the art. These intermediates can serve as alkylating agents for amines such as (but not limited to) 1 and 2 to afford targets 22-25.

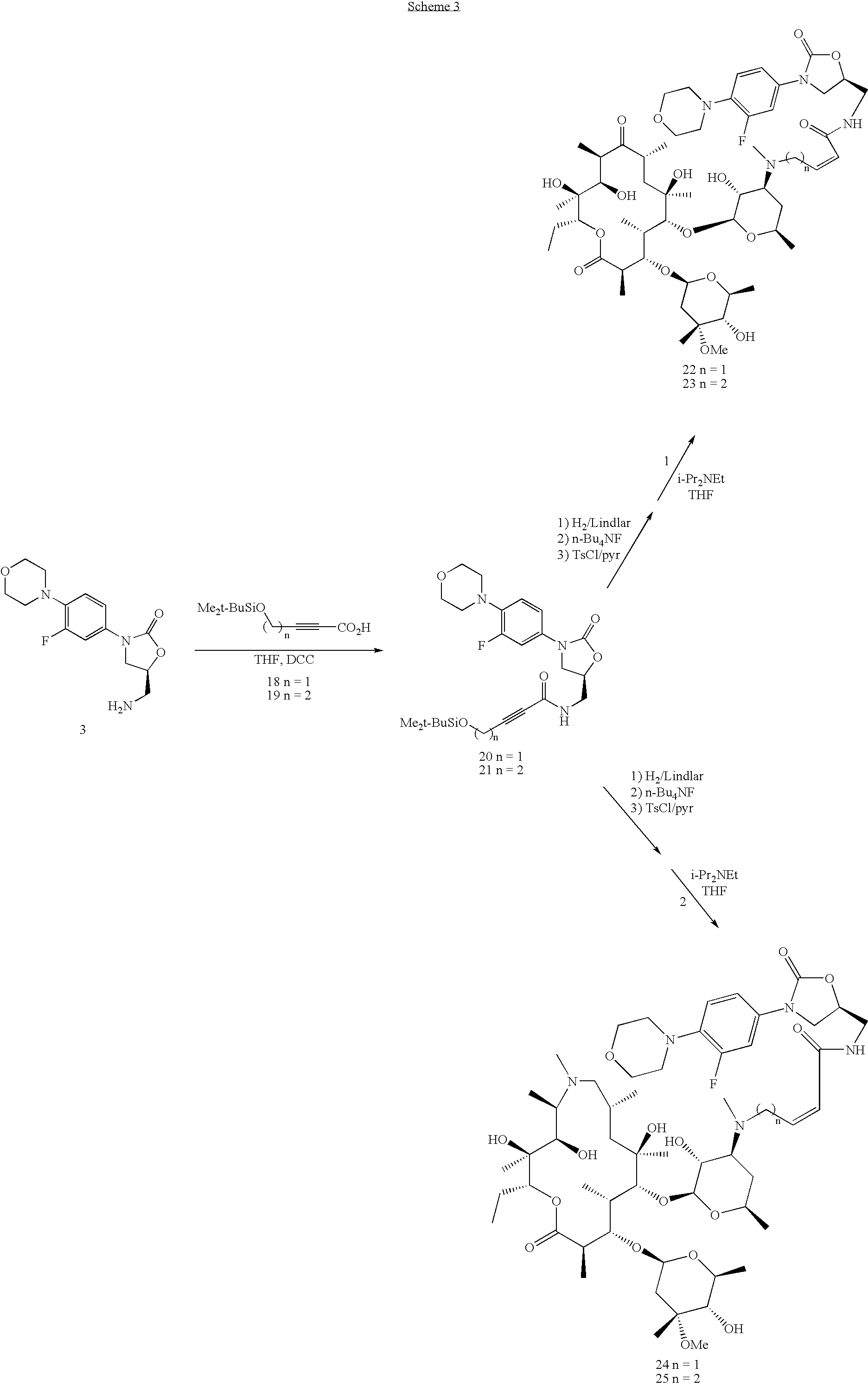

An alternate approach to targets such as 22-25 is shown in Scheme 4. The siloxy groups of alkynes 20 and 21 can be converted to tosylates to serve as alkylating agents for amines 1 and 2. The products of the alkylation reaction can then be reduced to give the cis olefin as above to provide targets 22-25.

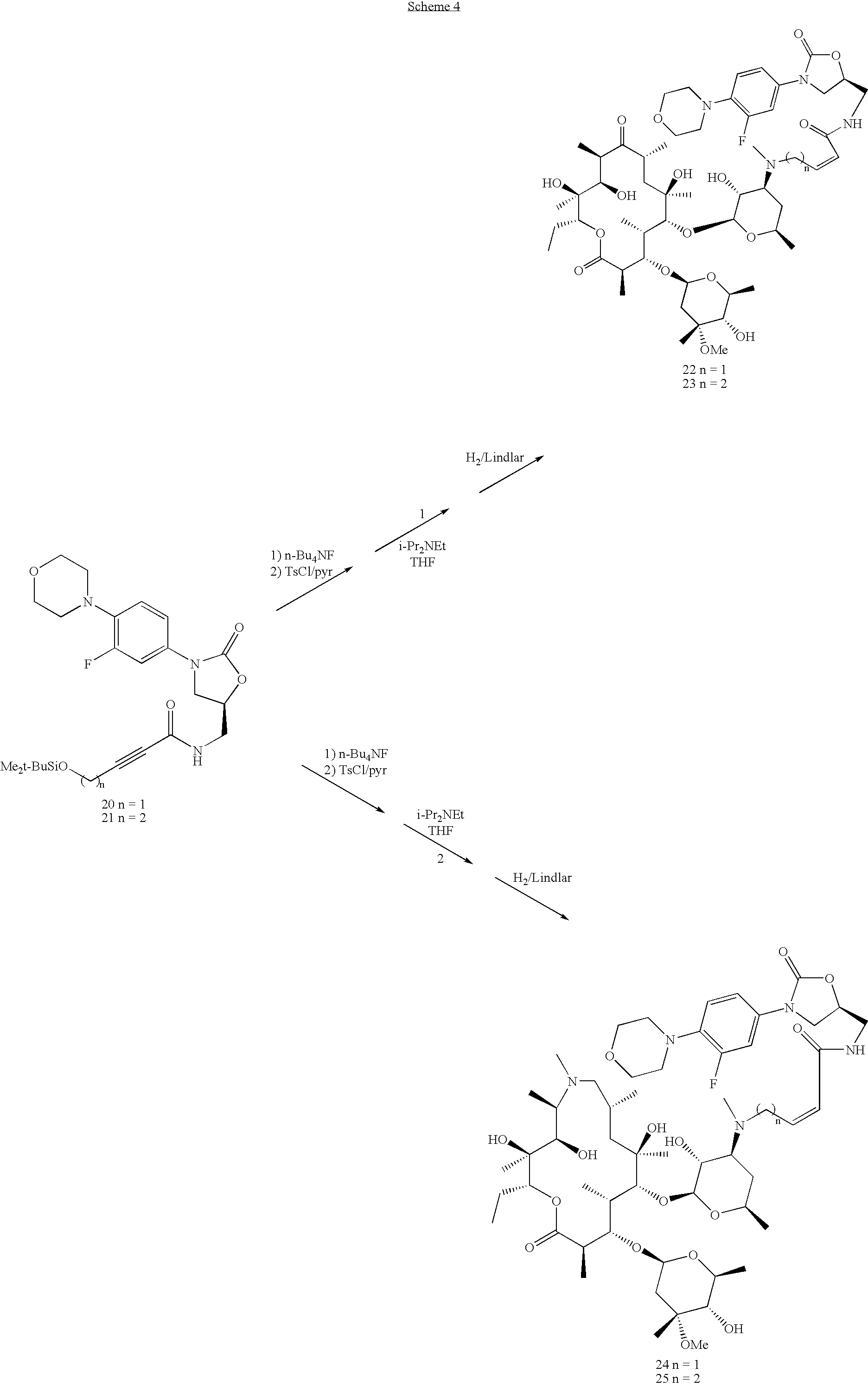

Scheme 5 illustrates how amines such as (but not limited to) 3 can be converted to compounds of the present invention containing linker groups of type L5. Amines 1 and 2 can be alkylated with ω-bromo-1-alkanols to afford alcohols such as 26-29. Isocyanates similar to 30 can be made from amines such as 3 using phosgene, carbonyldiimidazole or other reagents well known to those skilled in the art. The reaction of the primary alcohols of compounds 26-29 with isocyanates such as 30 will lead to carbamate targets such as 31-34. It will be understood that amines of type 3 could be converted to isothiocyanates corresponding to 30, and that these intermediates could be used to make thiocarbamate derivatives related to 31-34, containing linker groups of type L6. It will be further appreciated that the primary alcohols of intermediates such as 26-29 could be converted by chemistry well known in the art to primary amines which could then be used to react with isocyanate 30 or the corresponding isothiocyanate to provide urea- and thiourea-linked compounds of the present invention. It is intended that such urea and thiourea derivatives are included in the scope of the present invention. 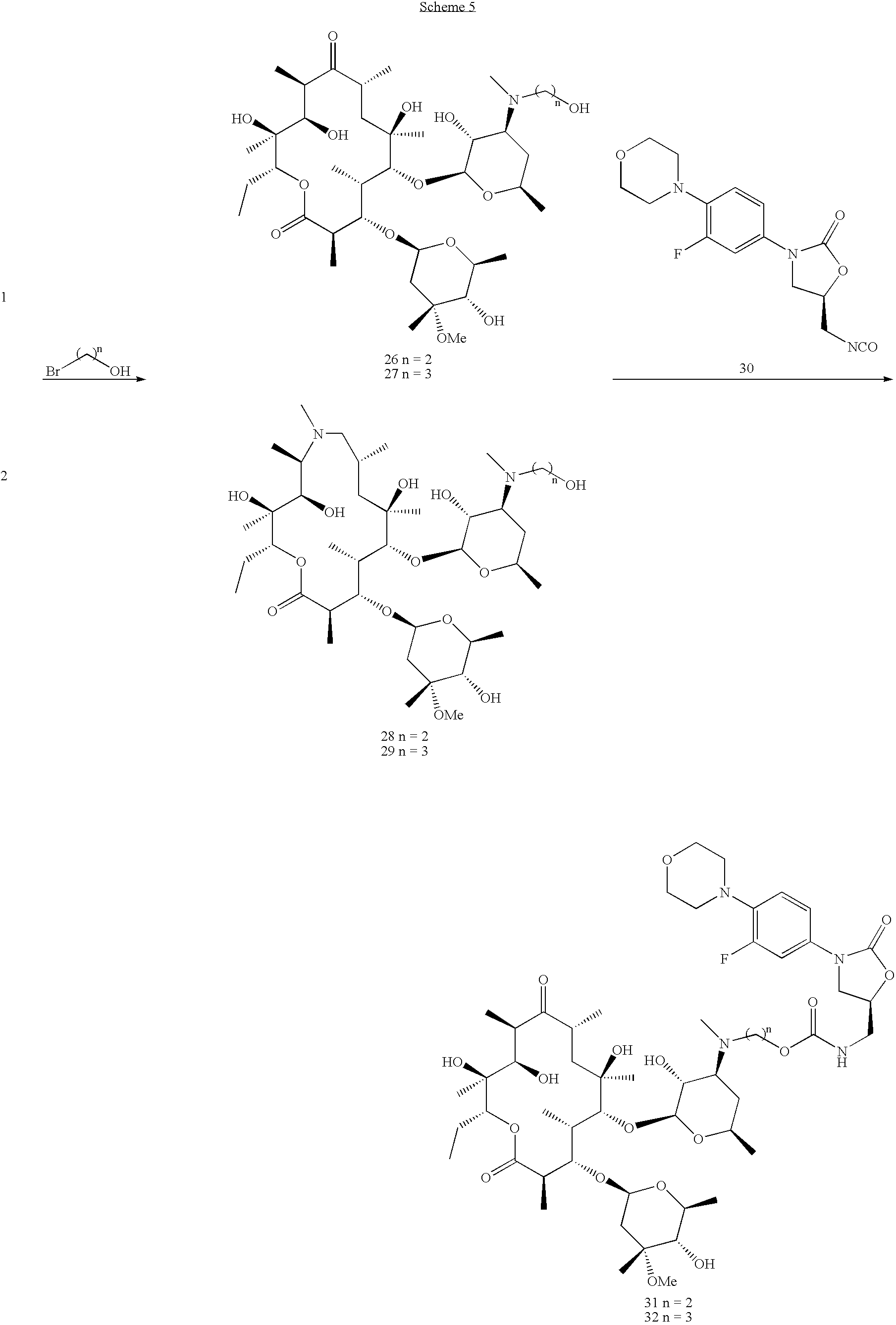 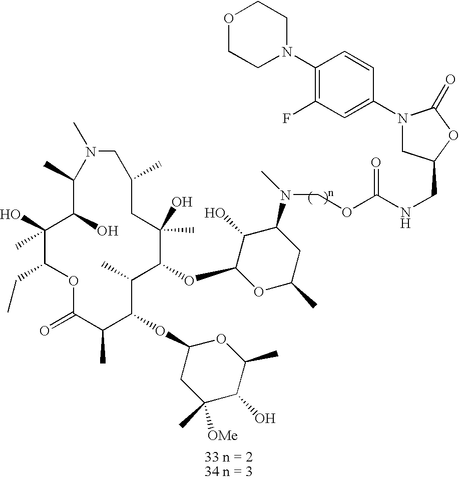
4. Characterization of Compounds of the Invention

Compounds designed, selected and/or optimized by methods described above, once produced, may be characterized using a variety of assays known to those skilled in the art to determine whether the compounds have biological activity. For example, the molecules may be characterized by conventional assays, including but not limited to those assays described below, to determine whether they have a predicted activity, binding activity and/or binding specificity.

Furthermore, high-throughput screening may be used to speed up analysis using such assays. As a result, it may be possible to rapidly screen the molecules described herein for activity, for example, as anti-cancer, anti-bacterial, anti-fungal, anti-parasitic or anti-viral agents. Also, it may be possible to assay how the compounds interact with a ribosome or ribosomal subunit and/or are effective as modulators (for example, inhibitors) of protein synthesis using techniques known in the art. General methodologies for performing high-throughput screening are described, for example, in Devlin (1998) High Throughput Screening, Marcel Deicker; and U.S. Pat. No. 5,763,263. High-throughput assays can use one or more different assay techniques including, but not limited to, those described below.

(1) Surface Binding Studies. A variety of binding assays may be useful in screening new molecules for their binding activity. One approach includes surface plasmon resonance (SPR) that can be used to evaluate the binding properties of molecules of interest with respect to a ribosome, ribosomal subunit or a fragment thereof.

SPR methodologies measure the interaction between two or more macromolecules in real-time through the generation of a quantum-mechanical surface plasmon. One device, (BIAcore Biosensor RTM from Pharmacia Biosensor, Piscatawy, N.J.) provides a focused beam of polychromatic light to the interface between a gold film (provided as a disposable biosensor “chip”) and a buffer compartment that can be regulated by the user. A 100 nm thick “hydrogel” composed of carboxylated dextran that provides a matrix for the covalent immobilization of analytes of interest is attached to the gold film. When the focused light interacts with the free electron cloud of the gold film, plasmon resonance is enhanced. The resulting reflected light is spectrally depleted in wavelengths that optimally evolved the resonance. By separating the reflected polychromatic light into its component wavelengths (by means of a prism), and determining the frequencies that are depleted, the BIAcore establishes an optical interface which accurately reports the behavior of the generated surface plasmon resonance. When designed as above, the plasmon resonance (and thus the depletion spectrum) is sensitive to mass in the evanescent field (which corresponds roughly to the thickness of the hydrogel). If one component of an interacting pair is immobilized to the hydrogel, and the interacting partner is provided through the buffer compartment, the interaction between the two components can be measured in real time based on the accumulation of mass in the evanescent field and its corresponding effects of the plasmon resonance as measured by the depletion spectrum. This system permits rapid and sensitive real-time measurement of the molecular interactions without the need to label either component.

(2) Fluorescence Polarization. Fluorescence polarization (FP) is a measurement technique that can readily be applied to protein-protein, protein-ligand, or RNA-ligand interactions in order to derive IC₅₀s and Kds of the association reaction between two molecules. In this technique one of the molecules of interest is conjugated with a fluorophore. This is generally the smaller molecule in the system (in this case, the compound of interest). The sample mixture, containing both the ligand-probe conjugate and the ribosome, ribosomal subunit or fragment thereof, is excited with vertically polarized light. Light is absorbed by the probe fluorophores, and re-emitted a short time later. The degree of polarization of the emitted light is measured. Polarization of the emitted light is dependent on several factors, but most importantly on viscosity of the solution and on the apparent molecular weight of the fluorophore. With proper controls, changes in the degree of polarization of the emitted light depends only on changes in the apparent molecular weight of the fluorophore, which in-turn depends on whether the probe-ligand conjugate is free in solution, or is bound to a receptor. Binding assays based on FP have a number of important advantages, including the measurement of IC₅₀ss and Kds under true homogenous equilibrium conditions, speed of analysis and amenity to automation, and ability to screen in cloudy suspensions and colored solutions.

(3) Protein Synthesis. It is contemplated that, in addition to characterization by the foregoing biochemical assays, the compound of interest may also be characterized as a modulator (for example, an inhibitor of protein synthesis) of the functional activity of the ribosome or ribosomal subunit.

Furthermore, more specific protein synthesis inhibition assays may be performed by administering the compound to a whole organism, tissue, organ, organelle, cell, a cellular or subcellular extract, or a purified ribosome preparation and observing its pharmacological and inhibitory properties by determining, for example, its inhibition constant (IC₅₀) for inhibiting protein synthesis. Incorporation of ³H leucine or ³⁵S methionine, or similar experiments can be performed to investigate protein synthesis activity. A change in the amount or the rate of protein synthesis in the cell in the presence of a molecule of interest indicates that the molecule is a modulator of protein synthesis. A decrease in the rate or the amount of protein synthesis indicates that the molecule is a inhibitor of protein synthesis.

Furthermore, the compounds may be assayed for anti-proliferative or anti-infective properties on a cellular level. For example, where the target organism is a microorganism, the activity of compounds of interest may be assayed by growing the microorganisms of interest in media either containing or lacking the compound. Growth inhibition may be indicative that the molecule may be acting as a protein synthesis inhibitor. More specifically, the activity of the compounds of interest against bacterial pathogens may be demonstrated by the ability of the compound to inhibit growth of defined strains of human pathogens. For this purpose, a panel of bacterial strains can be assembled to include a variety of target pathogenic species, some containing resistance mechanisms that have been characterized. Use of such a panel of organisms permits the determination of structure-activity relationships not only in regards to potency and spectrum, but also with a view to obviating resistance mechanisms. The assays may be performed in microtiter trays according to conventional methodologies as published by The National Committee for Clinical Laboratory Standards (NCCLS) guidelines (NCCLS. M7-A5-Methods for Dilution Antimicrobial Susceptibility Tests for Bacteria That Grow Aerobically; Approved Standard-Fifth Edition. NCCLS Document M100-S12/M7 (ISBN 1-56238-394-9)).

5. Formulation and Administration

The compounds of the invention may be useful in the prevention or treatment of a variety of human or other animal disorders, including for example, bacterial infection, fungal infections, viral infections, parasitic diseases, and cancer. It is contemplated that, once identified, the active molecules of the invention may be incorporated into any suitable carrier prior to use. The dose of active molecule, mode of administration and use of suitable carrier will depend upon the intended recipient and target organism. The formulations, both for veterinary and for human medical use, of compounds according to the present invention typically include such compounds in association with a pharmaceutically acceptable carrier.

The carrier(s) should be “acceptable” in the sense of being compatible with the other ingredients of the formulations and not deleterious to the recipient. Pharmaceutically acceptable carriers, in this regard, are intended to include any and all solvents, dispersion media, coatings, anti-bacterial and anti-fungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations may conveniently be presented in dosage unit form and may be prepared by any of the methods well known in the art of pharmacy/microbiology. In general, some formulations are prepared by bringing the compound into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, for example, intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences (Gennaro, A., ed.), Mack Pub., (1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or moulding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Moulded tablets may be made by moulding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients.

Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystalline form, for example, in the form of an aqueous microcrystalline suspension. Liposomal formulations or biodegradable polymer systems may also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect may be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in therapeutically effective amounts, e.g., amounts that provide appropriate concentrations of the drug to target tissue for a time sufficient to induce the desired effect.

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

Active compound as identified or designed by the methods described herein can be administered to individuals to treat disorders (prophylactically or therapeutically). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharnacogenomics studies in determining whether to administer a drug as well as tailoring the dosage and/or therapeutic regimen of treatment with the drug.

In therapeutic use for treating, or combating, bacterial infections in mammals, the compounds or pharmaceutical compositions thereof will be administered orally, parenterally and/or topically at a dosage to obtain and maintain a concentration, that is, an amount, or blood-level or tissue level of active component in the animal undergoing treatment which will be anti-microbially effective. The term “effective amount” is understood to mean that the compound of the invention is present in or on the recipient in an amount sufficient to elicit biological activity, for example, anti-microbial activity, anti-fungal activity, anti-viral activity, anti-parasitic activity, and/or anti-proliferative activity. Generally, an effective amount of dosage of active component will be in the range of from about 0.1 to about 100, more preferably from about 1.0 to about 50 mg/kg of body weight/day. The amount administered will also likely depend on such variables as the type and extent of disease or indication to be treated, the overall health status of the particular patient, the relative biological efficacy of the compound delivered, the formulation of the drug, the presence and types of excipients in the formulation, and the route of administration. Also, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

### EXAMPLES

In the following examples, nuclear magnetic resonance (NMR) spectra were obtained on a Bruker Avance 300 or Avance 500 spectrometer, or in some cases a GE-Nicolet 300 spectrometer. Common reaction solvents were either high performance liquid chromatography (HPLC) grade or American Chemical Society (ACS) grade, and anhydrous as obtained from the manufacturer unless otherwise noted. “Chromatography” or “purified by silica gel” refers to flash column chromatography using silica gel (EM Merck, Silica Gel 60, 230-400 mesh) unless otherwise noted.

### Example 1

### Exemplary Compounds

Exemplary compounds synthesized in accordance with the invention are listed in Table 1.

TABLE 1

| Compound Number | Structure |
|---|---|
| 6 | 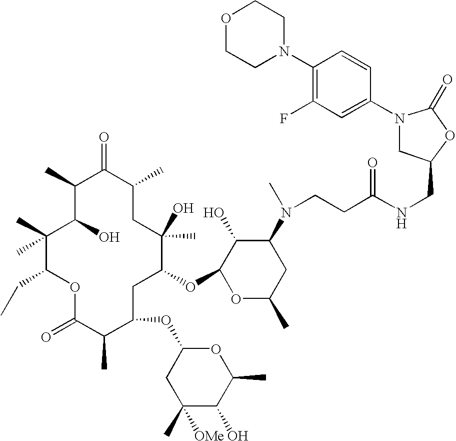 |
| 7 | 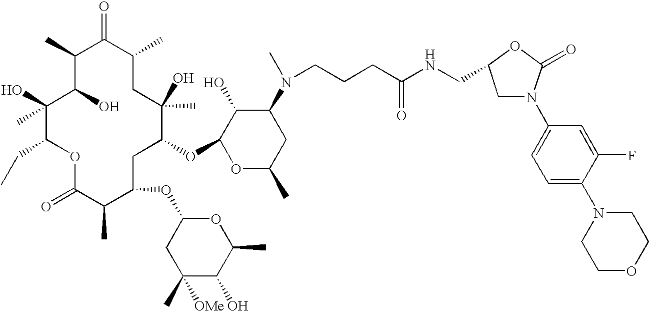 |
| 8 | 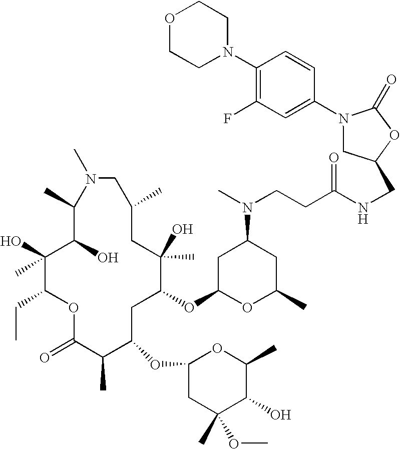 |
| 9 | 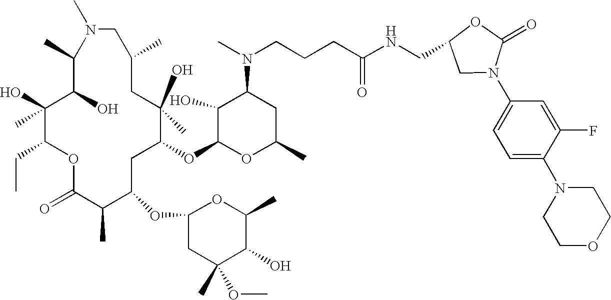 |
| 35 | 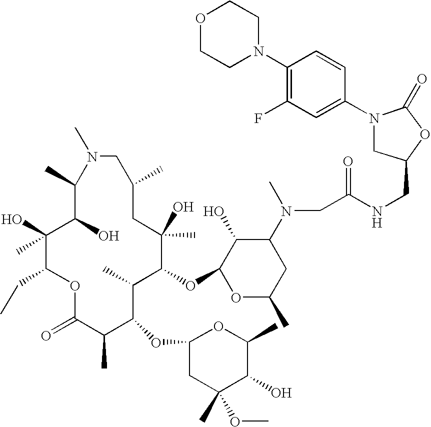 |
| 37 | 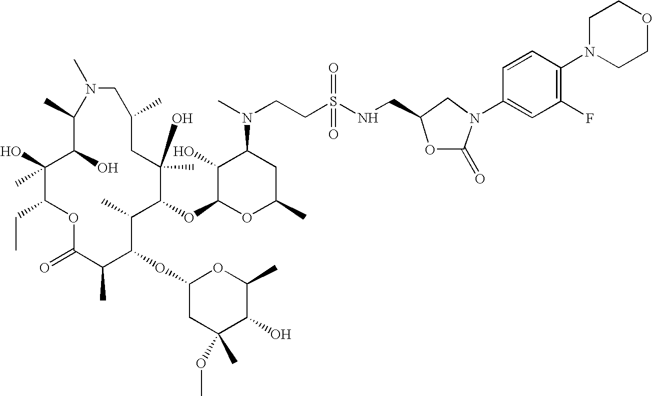 |
| 38 | 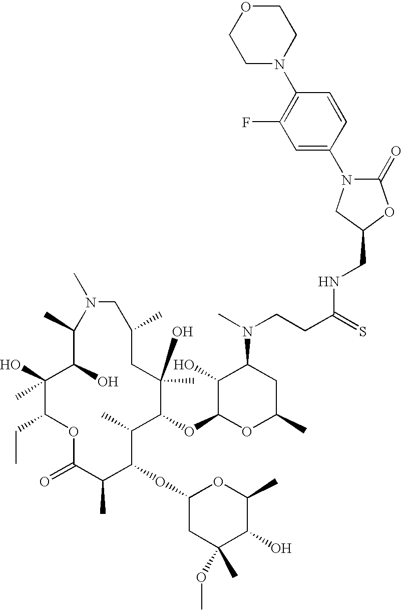 |
| 39 | 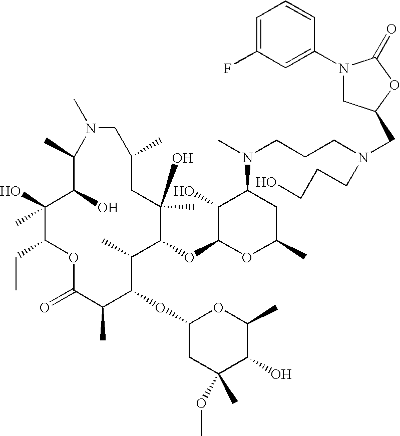 |
| 102 | 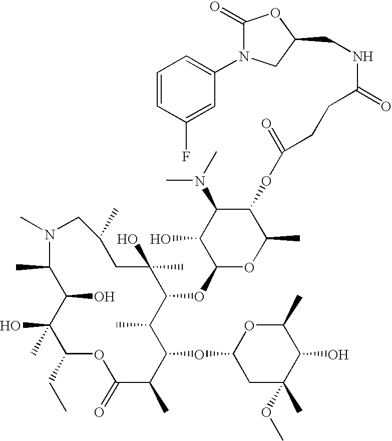 |
| 103 | 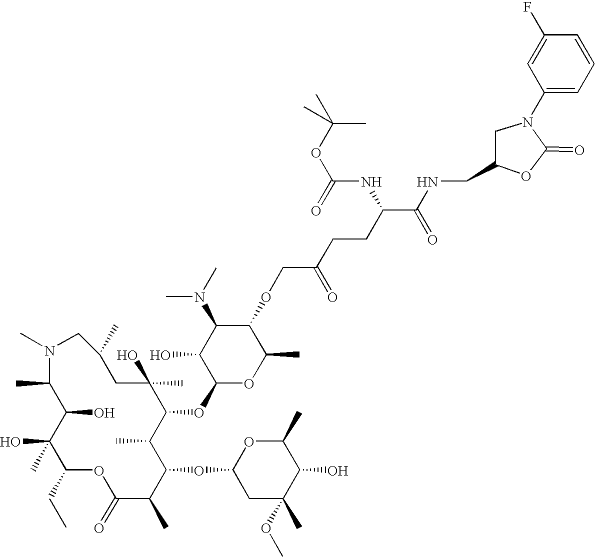 |

### Example 2

### Synthesis of Compounds 6-9

Synthesis of Amine 2

Azithromycin (0.80 g, 1.02 mmol) and sodium acetate (NaOAc) (0.712 g, 8.06 mmol) were dissolved in 80% aqueous methanol (MeOH) (25 mL). The solution was kept at 50° C. followed by addition of iodine (I₂) (0.272 g, 1.07 mmol) in three batches within 3 minutes (min). The reaction was maintained at a pH between 8-9 by adding 1N sodium hydroxide (NaOH) (1 mL) at 10 min and 45 min intervals. The solution turned colorless within 45 min, however, stirring was continued for 2 hours (hr). TLC (methylene chloride/methanol/ammonium hydroxide (CH₂Cl₂/MeOH/NH₄OH) 10:1:0.05) after 2 hr showed a single major product (Rf=0.66). The reaction was cooled to room temperature (RT), poured into water (H₂O) (75 mL) containing NH₄OH (1.5 mL) and extracted with chloroform (CHCl₃) (3×30 mL). The combined organic layer was washed with H₂O (30 mL) containing NH₄OH (1.5 mL), dried over sodium sulfate (Na₂SO₄) and the solvent evaporated to give a white residue. The crude was purified on silica gel column eluting with CH₂Cl₂/MeOH/NH₄OH 18:1:0.05 to 10:1:0.05 to provide amine 2 (0.41 g, 55% yield).

Synthesis of Bromide 4

To a solution of amine 3 (0.43 g, 1.46 mmol) in anhydrous tetrahydrofuran (THF) (10 mL) was added 3-bromopropionic acid (0.23 g, 1.5 mmol) and 1,3-dicyclohexylcarbodiimide (DCC) (0.36 g, 1.72 mmol). A precipitate developed within 2 min. Stirring was continued at RT for 2 hr, and the reaction was then filtered to remove the precipitated urea. CH₂Cl₂ (60 mL) and saturated ammonium chloride (NH₄Cl) were added to the filtrate. The two layers were separated, and the organic layer was washed with saturated NH₄Cl (2×30 mL) then saturated brine (1×30 mL), and dried over Na₂SO₄. The solvent was evaporated, and the residue was purified on a silica gel column eluting with ethyl acetate (EtOAc)/Hexanes 8:1 to 9:1 to give 4 as a white solid (0.42 g, 67% yield). ¹H-NMR (300 MHz, deuterated chloroform (CDCl₃); partial): δ 7.30 (dd, J=14, 3 Hz, 1H), 6.95 (dd, J=9, 2 Hz, 1H), 6.78 (t, J=9 Hz, 1H), 6.19 (t, J=6 Hz, 1H), 4.67 (m, 1H), 3.45-3.89 (m, 10H), 2.92 (t, J=5 Hz, 4H), 2.65 (m, 2H)

Synthesis of Bromide 5

Compound 5 was synthesized from amine 3 and 4-bromobutyric acid as described for amide 4. ¹H-NMR (300 MHz, CDCl₃; partial): δ 7.36 (dd, J=14, 3 Hz, 1H), 7.01 (dd, J=9, 2 Hz, 1H), 6.85 (t, J=9 Hz, 1H), 5.95 (t, J=6 Hz, 1H), 4.69 (m, 1H), 3.79 (t, J=5 Hz, 4H), 3.57-3.70 (m, 4H), 3.33 (t, J=6 Hz, 2H), 2.92 (t, J=5 Hz, 4H), 2.32 (m, 2H), 2.05 (m, 2H).

Synthesis of Compound 8

A mixture of amine 2 (0.128 g, 0.174 mmol) and bromide 4 (0.12 g, 0.28 mmol) in anhydrous THF (15 mL) and N,N-diisopropylethylamine (Hunig's base or i-Pr₂NEt) (3 mL) was heated under reflux overnight. The reaction was poured into CH₂Cl₂ (60 mL), washed with saturated NH₄Cl (3×30 mL) then saturated brine (1×30 mL), and dried over Na₂SO₄. The solvent was evaporated, and the crude material was purified on silica gel column first eluting with 2-6% MeOH in CH₂Cl₂ then with CH₂Cl₂/MeOH/NH₄OH 15:1: 0.05 to 12:1:0.05 to 10:1 to give compound 8 (0.0392 g, 21% yield). ¹H-NMR (300 MHz, CDCl₃; partial): δ 9.01 (bs, 1H), 7.46 (dd, J=14, 2 Hz, 1H), 7.05 (dd, J=9, 2 Hz, 1H), 6.91 (t, J=9 Hz, 1H), 5.10 (bs, 1H), 4.72 (m, 3H), 4.41 (d, J=7 Hz, 1H), 4.26 (bs, 1H), 3.99 (m, 2H), 3.86 (t, J=5 Hz, 4H), 3.05 (t, J=5 Hz, 4H).

Synthesis of Compound 9

Compound 9 was made from amine 2 and bromide 5 as described above for compound 8. ¹H-NMR (300 MHz, CDCl₃; partial): δ 8.99 (bs, 1H), 7.40 (dd, J=14, 3 Hz, 1H), 7.05 (dd, J=9, 2 Hz, 1H), 6.87 (t, J=9 Hz, 1H), 5.02 (d, J=2 Hz, 1H), 4.85 (m, 1H), 4.34 (d, J=7 Hz, 1H), 3.82 (t, J=4 Hz, 4H), 3.01 (t, J=4 Hz, 4H).

Synthesis of Compound 6

Compound 6 was made from amine 1 and amide 4 as described above for compound 8. ¹H-NMR (300 MHz, CDCl₃; partial): δ 8.97 (t, J=6 Hz, 1H), 7.39 (dd, J=14, 3 Hz, 1H), 7.04 (dd, J=9, 2 Hz, 1H), 6.87 (t, J=9 Hz, 1H), 5.01 (d, J=2 Hz, 1H), 4.84 (d, J=4 Hz, 1H), 4.69 (m, 1H), 4.33 (d, J=7 Hz, 1H), 3.82 (t, J=4 Hz, 4H), 3.01 (t, J=5 Hz, 4H).

Synthesis of Compound 7

Compound 7 was made from amine 1 and amide 5 as described above for compound 8. ¹H-NMR (300 MHz, CDCl₃; partial): δ 8.99 (bs, 1H), 7.40 (dd, J=14, 3 Hz, 1H), 7.05 (dd, J=9, 2 Hz, 1H), 6.87 (t, J=9 Hz, 1H), 5.02 (d, J=3 Hz, 1H), 4.85 (m, 1H), 4.69 (m, 1H), 4.34 (d, J=7 Hz, 1H), 3.82 (t, J=4 Hz, 4H),3.01 (t, J=4 Hz, 4H).

### Example 3

### Synthesis of Compound 35

Synthesis of Bromide 36

Compound 36 was synthesized from amine 3 and bromoacetic acid as described for amide 4. ¹H-NMR (300 MHz, CDCl₃; partial): δ 7.36 (dd, J=11, 2 Hz, 1H), 7.02 (m, 2H), 6.93 (dd, J=9, 2 Hz, 1H), 4.68 (m, 1H), 3.93-3.53 (m, 10H), 3.01 (t, J=5 Hz, 4H). LCMS (ESI) m/z 418 (M+H⁺).

Synthesis of Compound 35

Compound 35 was made from amine 2 and amide 36 as described for compound 8. ¹H-NMR (300 MHz, CDCl₃; partial): δ 8.25 (t, J=6 Hz, 1H), 7.40 (dd, J=14, 3 Hz, 1H), 6.99 (dd, J=9, 2Hz, 1H), 6.84 (t, J=9Hz, 1H), 4.96(d, J=4Hz, 1H), 4.69 (m, 2H), 4.31 (d, J=8 Hz, 2H), 4.19 (bs, 1H), 3.80 (t, J=5 Hz, 4H),2.98 (t, J=5 Hz, 4H). LCMS (ESI) m/z 1070 z (M+H⁺).

### Example 4

### Synthesis of Sulfonamide 37

Scheme 6 shows the synthesis of sulfonamide 37. The hydrochloride salt (41) of the known amine (see: Barbachyn, M. et al. International Patent Application WO95/07271) was converted to sulfonamide 42. Sulfonamide 42 was used to alkylate amine 171 to afford sulfonamide 37. 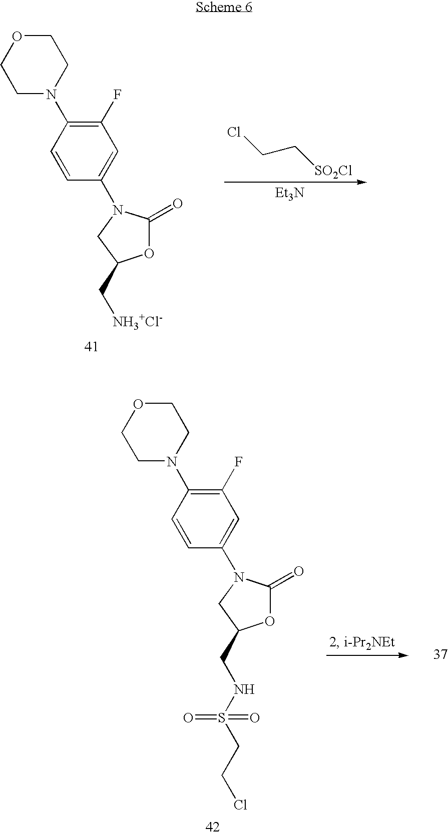
Synthesis of Sulfonamide 42

To a stirred solution of amine salt 41 (332 mg, 1.0 mmol) in CH₂Cl₂ was added sequentially triethylamine (Et₃N) (0.5 mL), and 2-chloro-ethanesulfonyl chloride (ClCH₂CH₂SO₂Cl) (163 mg, 1.0 mmol). The reaction mixture was stirred at (T) for 16 hr, then diluted to 50 mL with CH₂Cl₂ and washed with saturated aqueous sodium bicarbonate (NaHCO₃) (50 m L) and brine (25 mL). The organic fraction was dried over potassium carbonate (K₂CO₃), filtered and concentrated to give 490 mg of a yellow solid which was purified by silica gel chromatography (15 mm×6″ column eluted with 1:1 acetone/hexanes) to afford sulfonamide 42 as a colorless solid (380 mg, 0.90 mmol) which was used as-is for the next reaction.

Synthesis of Sulfonamide 37

To a stirred solution of amine 2 (0.10 g, 0.14 mmol) in 3 mL i-Pr₂NEt was added 42 (0.10 g, 0.24 mmol). The solution was sealed in a 1 dram vial and heated in a 100° C. oil bath for 16 h. The reaction mixture was cooled, then diluted to 50 mL with CH₂Cl₂ and washed with saturated aqueous NaHCO₃ (50 mL) and brine (25 mL). The organic fraction was dried over K₂CO₃, filtered and concentrated to give 170 mg of a yellow oil which was purified by silica gel chromatography (15 mm×6″ column eluted with 33:1 CH₂Cl₂/2N NH₃ in MeOH) to afford sulfonamide 37 as a white solid (105 mg, 0.094 mmol). ¹HNMR (300 MHz, CDCl₃; partial): δ 7.46 (dd, J=14,2 Hz, 1H), 7.06 (t, J=6Hz, 1H), 6.97 (dd, J=9, 2 Hz, 1H), 6.84 (t, J=9 Hz, 1H), 4.83 (d, J=2 Hz, 1 H), 4.58 (d, J=8 Hz, 1H), 4.38 (d, J=7 Hz, 1H), 4.15 (d, J=6 Hz, 1H), (3.22, s, 3H), 2.23 (s, 3H), 2.31 (s, 3H), 1.34-1.27 (m, 8H), 1.27-1.15 (m, 10H), 1.10-0.99 (m, 9H), 0.89-0.78 (m, 6H). LCMS (ESI) m/z 1120 (M+H)⁺, 560.8 (M+2H)²⁺.

### Example 5

### Synthesis of Compound 38

Scheme 7 depicts the synthesis of thioamide 38. Bromide 4 was converted to a mixture of thioamides 43 and 44, which was subsequently used to alkylate amine 2 to afford thioamide 38. 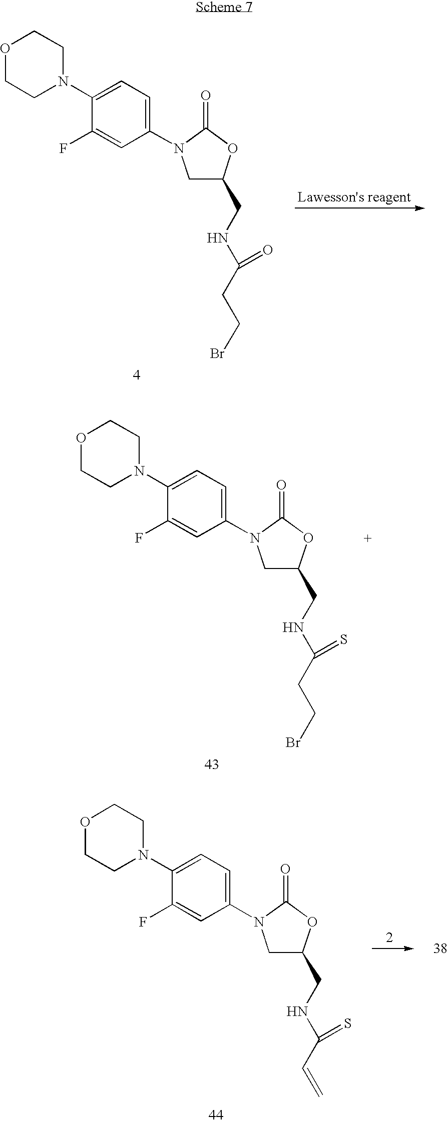
Synthesis of Thioamides 43 and 44

Bromopropionamide 4 (0.190 g, 0.440 mmol) and Lawesson's reagent (2,4-bis(4-methoxyphenyl)-1,3-dithia-2,4-diphosphetane-2,4-disulfide) (0.092 g, 0.220 mmol) were dissolved in anhydrous THF (10 mL) and the solution was heated under reflux for 2 h. The solvent was evaporated; and the crude was purified on a silica gel column, eluting with EtOAc/Hexanes (1:1 to 2:1) to give a brown-white solid. LCMS analysis showed that the isolated product was approximately a 7:3 mixture of thioamide 43 [m/e 448.1 (M+H)⁺] and thioamide 44 [m/e 366.0 (M+H)⁺] respectively (0.072 g, combined yield).

Synthesis of Thioamide 38

A mixture of amine 2 (0.250 g, 0.323 mmol) and thioamides 43 and 44 (0.072 g, 0.160 mmol based on 43) in anhydrous THF (5 mL) and Hunig's base (5 mL) was heated at 100° C. for 12 h. The reaction was poured into CH₂Cl₂ (60 mL), extracted with a mixture containing saturated NH₄Cl/NH₄OH (pH=9.5, 1×30 mL) and saturated brine (1×30 mL) and the organic phase was dried over Na₂SO₄. The solvent was evaporated and the crude was purified on a silica gel column first eluting with CH₂Cl₂/MeOH (18:1) then with CH₂Cl₂/MeOH/NH₄OH (18:1:0.03 to 18:1:0.05 to 15:1:0.05) to give 38 as a white solid (0.027 g, 15% yield). ¹H-NMR (300 MHz, CDCl₃; partial): δ 11.56 (bs, 1H), 7.40 (dd, J=14, 2 Hz, 1H), 6.95 (dd, J=9, 3 Hz, 1h), 6.83 (t, J=9 Hz, 1H), 4.93 (m, 2H), 4.61 (m, 1H), 4.33 (d, J=7 Hz, 1H), 3.78 (t, J=5 Hz, 4H), 2.96 (t, J=5 Hz, 4H). LCMS (ESI) m/z 1100.4 (M+H)⁺.

### Example 6

### Synthesis of Compound 39

Scheme 8 illustrates the synthesis of amine 39. Amine 45 was alkylated with 3-bromopropanol, and the resulting diol 46 was converted to monotosylate 47. Alkylation of amine 2 with tosylate 47 yielded amine 39. 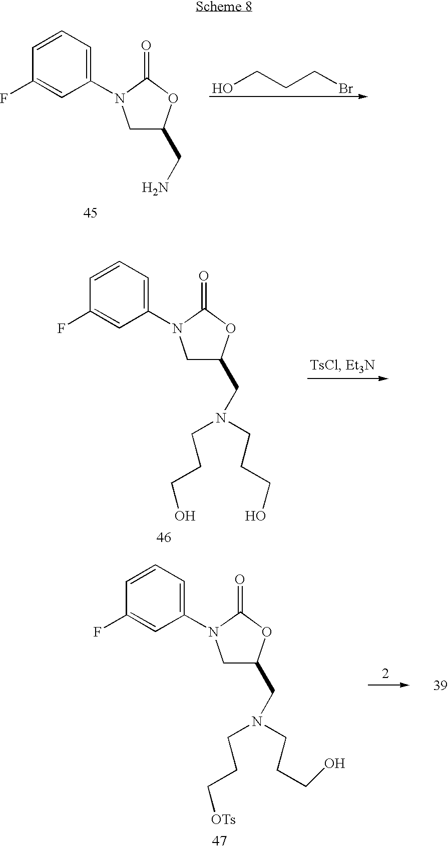
Synthesis of Diol 46

A mixture of amine 45 (0.50 g, 2.38 mmol; synthesized using the chemistry reported in the literature (azide synthesized via Brickner, S .J. et al. J Med Chem. 1996, 39, 673; then reduction of the azide to the amine) and 3-bromopropanol (0.42 mL, 4.68 mmol) in THF (10 mL) and i-Pr₂NEt (10 mL) was heated at 100° C. for 48 h. The solvent was evaporated and the crude was purified on a silica gel column, eluting with CH₂Cl₂/MeOH (14:1 to 12:1) to give alcohol 46 as thick oil (0.71 g, 91% yield). LCMS (EST) m/z 326.9 (M+H)⁺.

Synthesis of Tosylate 47

To a solution of alcohol 46 (0.35 g, 1.07 mmol) in CH₂Cl₂ (10 mL) and Et₃N (0.19 mL, 1.30 mmol) was added p-toluenesulfonyl chloride (TsCl) (0.21 g, 1.07 mmol) at 0° C. The reaction was allowed to warm to RT and stirred for 20 hr. The solvent was evaporated and the crude was purified on a silica gel column, eluting with CH₂Cl₂/MeOH (20:1 to 18:1 to 16:1) to give tosylate 47 (0.096 g, 19% yield). LCMS (ESI) m/z 481.0 (M+H)⁺.

Synthesis of Amine 39

A mixture of amine 2 (0.250 g, 0.323 mmol) and tosylate 47 (0.092 g, 0.192 mmol) in anhydrous THF (5 mL) and i-Pr₂NEt (5 mL) was heated at 100° C. for 24 hr. The solvent was evaporated and the crude was purified on a silica gel column eluting with CH₂Cl₂/MeOH/NH₄OH (18:1:0.04 to 16:1:0.04 to 14:1:0.05 to 12:1:0.05) to give amine 39 as a white solid (0.050 g, 25% yield). ¹H-NMR (300 MHz, CDCl₃; partial): δ 7.37 (m, 1H), 7.25 (m, 1H), 7.18 (m, 1H), 6.77 (m, 1H), 4.94 (d, J=4 Hz, 1H), 4.73 (m, 1H), 4.60 (d, F=9 Hz, 1H), 4.37 (d, J=7 Hz, 1H), 4.20 (d, J=4 Hz, 4H), 3.69 (m, 3H), 0.83 (m, 6H). LCMS (ESI) m/z 1043.6 (M+H)⁺.

### Example 7

### Synthesis of Compound 102

Scheme 9 illustrates the synthesis of compound 102. Amine 45 was condensed with succinic acid monomethyl ester to provide amide ester 110, which was hydrolyzed to carboxylic acid 111. This carboxylic acid 111 was condensed with 4′α-hydroxy-azithromycin 114, synthesis provided in Example 9, to yield compound 102. 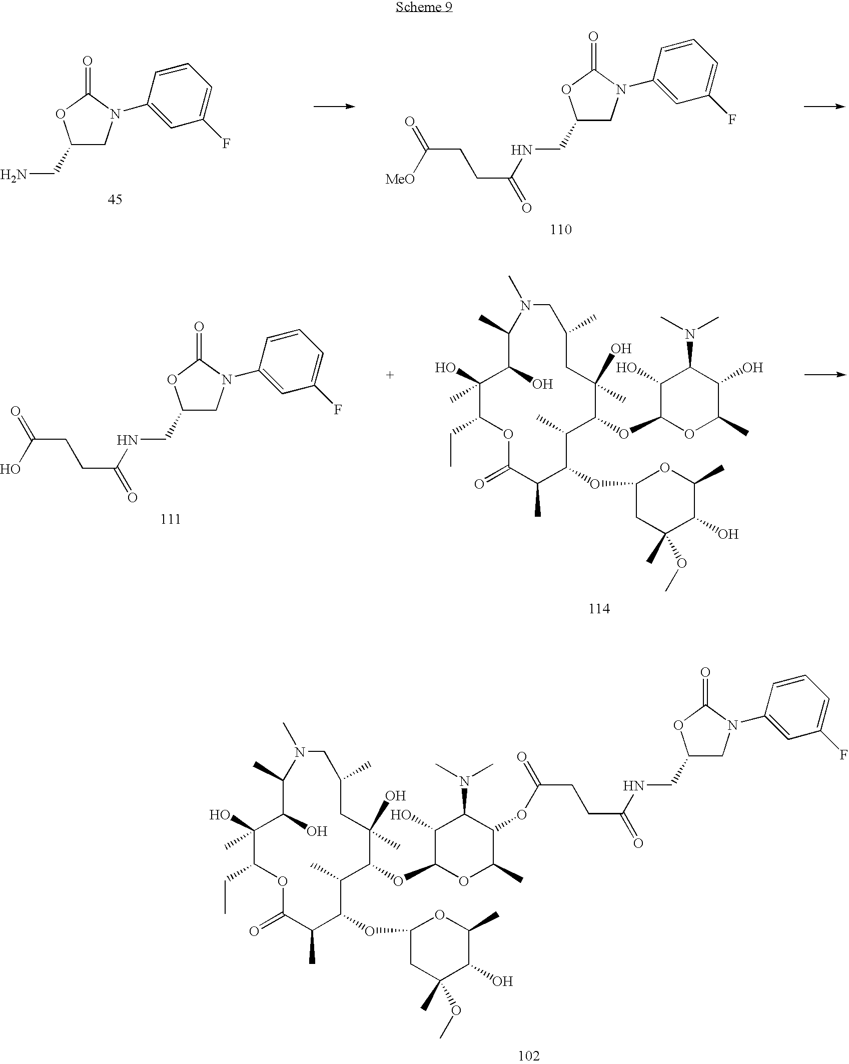
Synthesis of Amide Ester 110

To a solution of amine 45 (50 mg, 0.24 mmol), and succinic acid monomethyl ester (47 mg, 0.36 mmol) in 2 mL CH₂Cl₂, was added 74 mg DCC (0.36 mmol) and the mixture stirred at RT for 4 hr. The reaction mixture was placed directly on a silica gel column and eluted with hexane/acetone (3:1) to afford amide ester 110 (80 mg) as a white solid.

Synthesis of Carboxylic Acid 111

Amide ester 110 was dissolved in 5 mL ethanol (EtOH) and 0.5 mL 2N potassium hydroxide (KOH) (aq) was added. After stirring for 4 hr at RT the reaction mixture was diluted to 50 mL with 0.1N hydrochloric acid (HCl) (aq) and extracted with CH₂Cl₂ (3×30 mL). The combined organic fractions were dried over Na₂SO₄, filtered and concentrated to afford carboxylic acid 111 as a white solid.

Synthesis of Compound 102

To a stirred solution of this carboxylic acid 111 (24 mg, 0.07 mmol) and 4′-α-hydroxy azithromycin 2 (38 mg, 0.05 mmol) in 0.5 mL CH₂Cl₂ was added 76 μL of a 20% w/v solution of DCC in CH₂Cl₂. The mixture was stirred at RT for 15 hr then the entire reaction mixture was placed directly on a silica gel flash column and eluted with 1:20:0.1 MeOH/CH₂Cl₂/NH₃ to afford compound 102 as a white solid (41 mg, 0.04 mmol). LCMS (ESI) m/z 529.5 (M+2H)²⁺.

### Example 8

### Synthesis of Compound 103

Scheme 10 illustrates the synthesis of compound 103. Amine 45 was condensed with Boc-L-glutamic acid 5-benzyl ester to provide amide ester 112, which was hydrolyzed to carboxylic acid 113. This carboxylic acid 113 was condensed with amine 114 to yield compound 103. 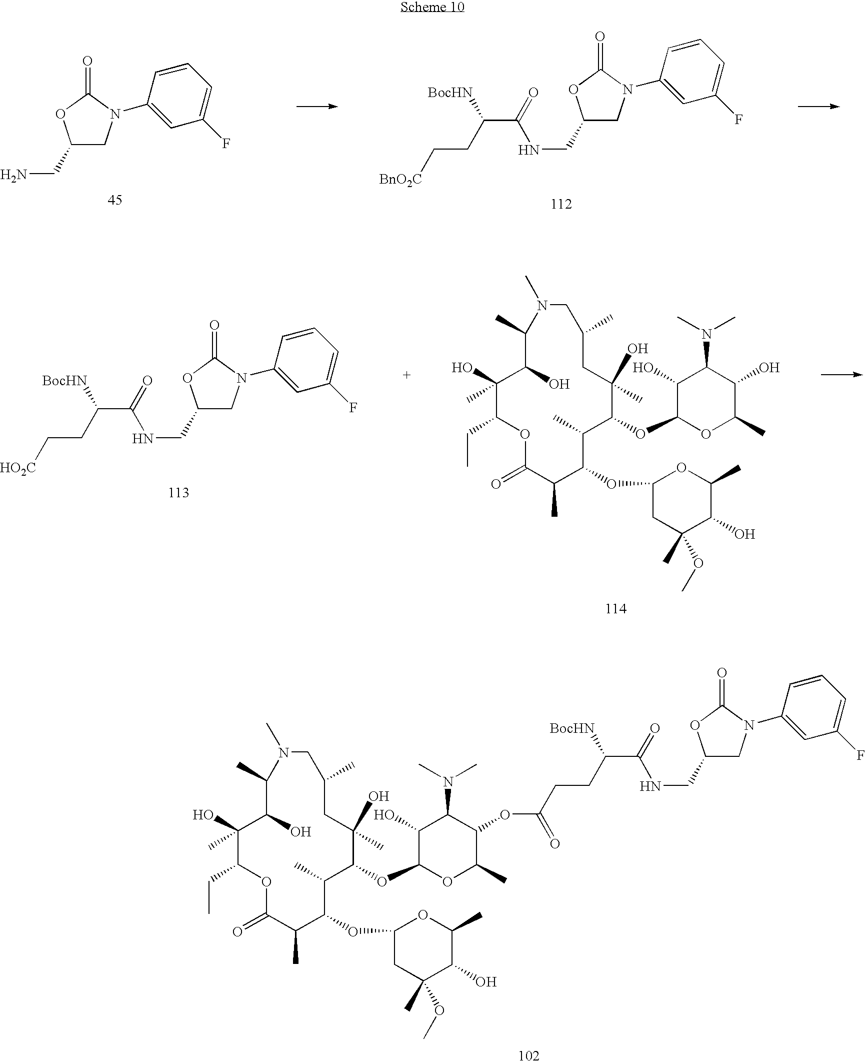
Synthesis of Amide Ester 112

To a solution of amine 45 (50mg, 0.24 mmol), and Boc-L-glutamic acid-5-benzyl ester (97 mg, 0.29 mmol) in 2 mL CH₂Cl₂, was added 74 mg DCC (0.36 mmol) and the mixture stirred at RT for 4 hr. The reaction mixture was placed directly on a silica gel column and eluted with hexane/acetone (4:1) to afford amide ester 112 (138 mg) as a white solid.

Synthesis of Carboxylic Acid 113

Amide ester 112 was dissolved in 5 mL EtOH and 0.5 mL 2N KOH(aq) was added. After stirring for 4 hr at RT the reaction mixture was diluted to 50 mL with 0.1N HCl(aq) and extracted with CH₂Cl₂ (3×30 mL). The combined organic fractions were dried over Na₂SO₄, filtered and concentrated to afford carboxylic acid 113 as a white solid (129 mg).

Synthesis of Compound 103

To a stirred solution of this carboxylic acid 113 (33 mg, 0.08 mmol) and 4′-α-hydroxy azithromycin 2 (38 mg, 0.05 mmol) in 0.5 mL CH₂Cl₂ was added 76 μL of a 20% w/v solution of DCC in CH₂Cl₂. The mixture was stirred at RT for 15 hr then the entire reaction mixture was placed directly on a silica gel flash column and eluted with 1:20:0.1 MeOH/CH₂Cl₂/NH₃ to afford compound 103 as a white solid (41 mg, 0.04 mmol). LCMS (ESI) m/Z 544.0 (M-Boc+2H)²⁺.

### Example 9

### Synthesis of Compound 114

Scheme 11 illustrates the synthesis of 4′α-hydroxy-azithromycin 114 from azithromycin 115 via amine oxide 116. The amide oxide 116 is converted to alkenyl compound 117, which is converted to epoxide N-oxide 118, which is subsequently converted to azido alcohol 119, and then to compound 114. 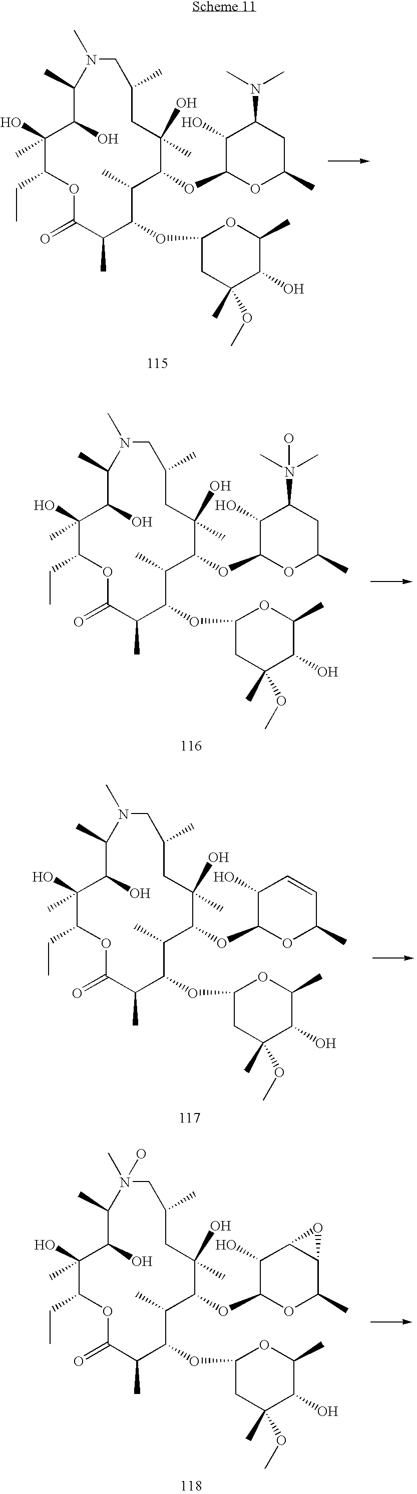 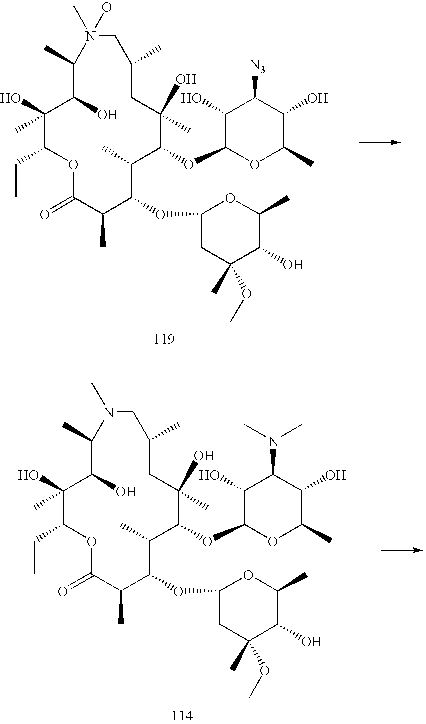
Synthesis of azithromycin-3′-N-oxide 116

Azithromycin 115 (50 g, 66.8 mmol) was dissolved in enough warm acetone to make 150 mL of solution. This solution was allowed to cool to ambient temperature prior to addition of 40 ml of 30% w/w aqueous H₂O₂. Following a mild exotherm, the solution was allowed to cool to ambient temperature and stirred for 3.5 hr. The reaction mixture was diluted to 2 L with CH₂Cl₂ and the resulting gelatinous mixture was stirred vigorously for 1 hr to afford a cloudy suspension. This suspension was washed with a 5:1 mixture of saturated aqueous NaHCO₃ and 10% w/v aqueous Na₂S₂O₃ (2×600 mL) and with brine (1×800 mL). The aqueous washes were combined and adjusted to pH 12 with 2N KOH and then further extracted with CH₂Cl₂ (3×300 mL). The combined organic extracts were dried over K₂CO₃, filtered, and concentrated in vacuo. As the volume of the extracts was reduced crystals began to form; when the total volume of the extracts had been reduced to 700 mL the solution was placed in a stoppered flask and stored at RT overnight. The solids were collected by vacuum filtration, rinsed with cold ether, and dried under vacuum to afford 34 g of white needle-like crystals. The filtrate was treated as before to yield two additional crops of crystalline product 201 for a total yield of 51 g (66.7 mmol 99%). LCMS (ESI) m/z 765.6 (M+H)⁺.

Synthesis of 3′ desdimethylamino4′-dehydro-azithromycin (alkenyl compound) 117

A 300 mL pear-shaped recovery flask was charged with Azithromycin-3′-N-oxide 116 (35 g, 45.8 mmol) and placed on a rotary evaporator. The pressure was reduced to 0.5 torr and the flask was rotated slowly in an oil bath while the temperature was gradually increased to 175° C. The mixture was held under vacuum at this temperature for 1.5 hr then cooled to RT and flushed with argon. The resulting tan solid was dissolved in 800 mL of boiling acetonitrile (CH₃CN). The solution was allowed to cool slowly to RT and then placed in a −20° C. freezer overnight. The solids were collected by vacuum filtration and washed with cold acetonitrile to afford 19.1 g of 117 as off-white crystals. The filtrate was concentrated and the residue treated as above to afford two additional crops of 117 product for a total yield of 27.7 g (39.4 mmol, 86%). LCMS (ESI) m/z 704.5 (M+H)⁺.

Synthesis of 3′ desdimethylamino-4′-dehydro-3′,4′-epoxy-9′N-oxo-azithromycin (epoxide N-oxide) 118

To a methanol solution of 117 (25.0 g, 35.5 mmol in 100 mL) was added meta-chloroperbenzoic acid (20.4 g, 89 mmol). The reaction mixture was stirred at RT for 14 hr at which time an additional 10 g portion of mCPBA was added. The solution was stirred for an additional 4 hr, then diluted with 1200 mL CH₂Cl₂ and washed with saturated aqueous NaHCO₃ (2×500 mL) and brine (1×500 mL). The aqueous washes were back-extracted with CH₂Cl₂ (2×500 mL). The combined organic extracts were dried on K₂CO₃, filtered, and concentrated to give a white foam (30.7 g) which was purified by silica gel chromatography (125 mm×6″ column eluted with 7.5% 2N ammonia (NH₃) in MeOH/CH₂Cl₂) to afford compound 118 as a white solid (25.7 g, 35.0 mmol, 98%). LCMS (ESI) m/z 779.6 (M+H)⁺.

Synthesis of 3′β-azido4′α-hydroxy-9′N-oxo-3′-desdimethylamino-azithromycin (azido alcohol) 119

Epoxide N-oxide 118 (20.0 g, 27.2 mmol) was dissolved in 88 mL of 10:1 dimethylsulfoxide-water (DMSO-H₂O) to which was added sodium azide (NaN₃) (17.7 g, 270 mmol) and magnesium perchlorate octahydrate (Mg(ClO₄)·8H₂O) (13.5 g, 40.8 mmol). The mixture was stirred under argon at 85° C. for 16 hr then cooled to RT and poured into saturated aqueous NaHCO₃ (1L) and extracted with CH₂Cl₂ (5×500 mL). The combined organic extracts were dried over K₂CO₃, filtered, and concentrated to afford a white foam (29 g). This material was dissolved in hot CH₃CN (1.2 L) and allowed to sit overnight at RT. The solids were filtered from the solution and rinsed with additional CH₃CN. The 8.7 g of crystalline solid thus obtained was confirmed by NMR and x-ray analysis to be pure 3′α-hydroxy-4′β-azido-9′N-oxo-3′-desdimethylamino-azithromycin formed by addition of the azide at the 4′ carbon of the epoxide. The mother liquors were concentrated and the residue again dissolved in boiling CH₃CN from which a second 3.0 g crop of the undesired isomer was obtained in pure form. The mother liquors, now enriched in the product 119, were concentrated and the residue purified by silica gel chromatography (50 mm×8″ column eluted with 0-8% 2N NH₃ in MeOH/CH₂Cl₂) to afford an additional 2.9 g of the earlier-eluting 4′β-azide along with compound 119 (6.5 g, 8.3 mmol, 31%). LCMS (ESI) m/z 736.6 (M+H)⁺.

Synthesis of 4′α-hydroxy-azithromycin 114

A heavy-walled pressure tube was charged with an ethanol solution of 119 (1.73 g, 2.22 mmol in 20 mL) and 20% palladium (Pd) on charcoal (C) (0.14 g containing 50% H₂O). The reaction mixture was stirred under an hydrogen (H₂) atmosphere (15 psig) at RT for 14 hr at which time 2 mL 37% aqueous formaldehyde (CH_{2 O}), 1 mL formic acid (HCO₂H), and an additional 50 mg Pd on C were added. The hydrogen pressure was increased to 30 psig and stirring was continued for 24 hr, at which time an additional 100 mg charge of Pd was added and the H₂ pressure was increased to 90 psig. After an additional 24 hr at this pressure the reaction mixture was purged with argon, filtered, diluted with 100 mL toluene, and concentrated in vacuo to afford 1.9 g of a colorless glass. The crude product was purified by silica gel chromatography (25 mm×6″ column eluted with 7% 2N NH₃ in MeOH/CH₂Cl₂) to afford compound 114 as a white solid (0.78 g, 1.0 mmol, 45%). LCMS (ESI) m/z 765.5 (M+H)⁺.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound having the formula: 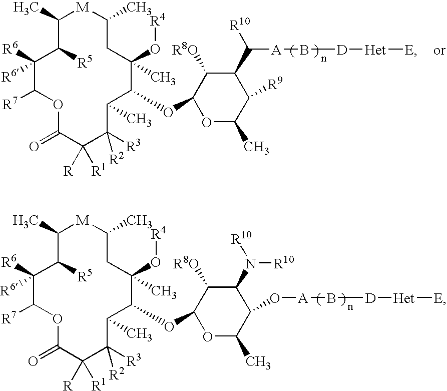
or pharmaceutically acceptable salt, ester or prodrug thereof,
wherein:
D-Het is selected from the group consisting of: 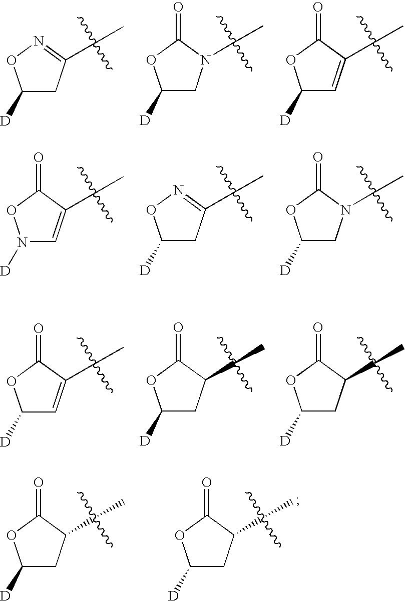
A is selected from the group consisting of:
a) carbonyl, b) C₁₋₆ alkyl, c) C₂₋₆ alkenyl d) —C(O)—C₁₋₆ alkyl, and e) —C(O)—C₂₋₆ alkenyl,
wherein
i) 0-2 carbon atoms of the C₁₋₆ alkyl and C₂₋₆ alkenyl groups in any of b)-e) optionally are replaced by a moiety selected from the group consisting of O, S(O)ₚ, and NR¹¹, and
ii) any of b)-e) optionally is substituted with one or more R¹² groups;
B is selected from the group consisting of:
a) —C(O)NH—, b) —C(S)NH—, c) —NHC(O)—, d) —NHC(S)—, e) —S(O)₂NH—, f) —NHS(O)₂—, g) —OC(O)NH—, h) —OC(S)NH—, i) —NHC(O)NH—, j) —NHC(S)NH—, k) —NHC(O)O—, 1) —NHC(S)O—, and m) —NR¹¹—;
n is 0 or 1;
D is selected from the group consisting of:
a) —CH₂—, b) —C(O)—, c) —C(S)—, d) —C(═NOR¹¹)—, e) —CH₂CH₂—, f) —OCH₂—g) —SCH₂—, h) —S(O)CH₂—, i) —S(O)₂CH₂—, j) —NR¹¹CH₂—, k) —C(O)CH₂—, l) —C(S)CH₂—, and m) —C(═NOR¹¹)CH₂—;
E is selected from the group consisting of: 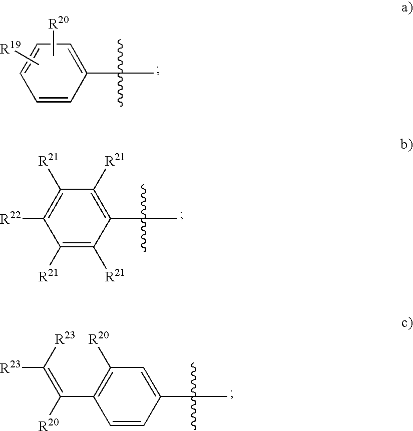
d) 5-10 membered saturated, unsaturated, or aromatic heterocycle containing one or more heteroatoms selected from the group consisting of nitrogen, oxygen, and sulfur, and optionally substituted with one or more R¹² groups;
e) C₅₋₁₀ saturated, unsaturated, or aromatic carbocycle, optionally substituted with one or more R¹² groups;
f) C₁₋₈ alkyl,
g) C₂₋₈ alkenyl,
h) C₂₋₈ alkynyl,
i) C₁₋₈ alkoxy,
j) C₁₋₈ alkylthio,
k) C₁₋₈ acyl,
l) S(O)ᵣR¹¹; and
m) hydrogen, wherein any of f)-k) optionally is substituted with
i) one or more R¹² groups;
ii) 5-6 membered saturated, unsaturated, or aromatic heterocycle containing one or more heteroatoms selected from the group consisting of nitrogen, oxygen, and sulfur, and optionally substituted with one or more R¹² groups; or
iii) C₅₋₁₀ saturated, unsaturated, or aromatic carbocycle, optionally substituted with one or more R¹² groups;
M is selected from the group consisting of:
a) —C(O)—, b) —C(═NOR¹¹)—, c) —CH(—OR¹¹)—, d) —NR¹¹—CH₂—, e) —CH₂—NR¹¹—, f) —CH(NR¹¹R¹¹)—, g) —C(═NNR¹¹R¹¹)—, h) —NR¹¹—C(O)—, i) —C(O)NR¹¹—, and j) —C(═NR¹¹)—;
R is selected from the group consisting of H and C₁₋₆ alkyl;
R¹ is selected from the group consisting of:
a) H, b) Cl, c) F, d) Br, e) I, f) —NR¹¹R¹¹ g) —NR¹¹C(O)R¹¹, h) —OR¹¹, i) —OC(O)R¹¹, j) —OC(O)OR¹¹, k) —OC(O)NR¹¹R¹¹, l) —O—C₁₋₆ alkyl-R¹², m) —OC(O)—C₁₋₆ alkyl-R¹², n) —OC(O)O—C₁₋₆ alkyl-R¹², o) —OC(O)NR¹¹—C₁₋₆ alkyl-R¹², p) C₁₋₆ alkyl, q) C₁₋₆ alkenyl, r) C₁₋₆ alkynyl,
wherein any of l)-r) optionally is substituted with one or more R¹² groups;
R² is H;
R³ is selected from the group consisting of:
a) H, b) —OR¹¹, c) —O—C₁₋₆ alkyl-R¹², d) —OC(O)R¹¹, e) —OC(O)—C₁₋₆ alkyl-R¹², f) —OC(O)OR¹¹, g) —OC(O)O—C₁₋₆ alkyl-R¹², h) —OC(O)NR¹¹R¹¹, i) —OC(O)NR¹¹—C₁₋₆ alkyl-R¹², and 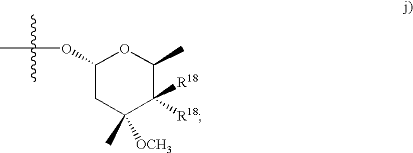
alternatively, R² and R³ taken together form a carbonyl group;
R⁴ is selected from the group consisting of:
a) H, b) R¹¹, c) —C(O)R¹¹ d) —C(O)OR¹¹ e) —C(O)NR¹¹R¹¹, f) —C₁₋₆ alkyl-G-R¹¹, g) —C₂₋₆ alkenyl-G-R¹¹, and h) —C₂₋₆ alkynyl-G-R¹¹;
alternatively R³ and R⁴, taken together with the atoms to which they are bonded, form: 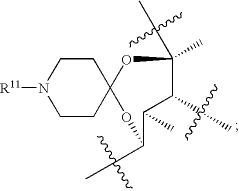
G is selected from the group consisting of:
a) —C(O)—, b) —C(O)O—, c) —C(O)NR¹¹—, d) —C(═NR¹¹)—, e) —C(═NR¹¹)O—, f) —C(═NR¹¹)NR¹¹—, g) —OC(O)—, h) —OC(O)O—, i) —OC(O)NR¹¹—, j) —NR¹¹C(O)—, k) —NR¹¹C(O)O—, l) —NR¹¹C(O)NR¹¹—, m) —NR¹¹C(═NR¹¹)NR¹¹—, and o) —S(O)ₚ—;
R⁵ is selected from the group consisting of:
a) R¹¹, b) —OR¹¹, c) —NR¹¹R¹¹, d) —O—C₁₋₆ alkyl-R¹², e) —C(O)—R¹¹, f) —C(O)—C₁₋₆ alkyl-R¹², g) —OC(O)—R¹¹, h) —OC(O)—C₁₋₆ alkyl-R¹², i) —OC(O)O—R¹¹, j) —OC(O)O—C₁₋₆ alkyl-R¹², k) —OC(O)NR¹¹R¹¹, l) —OC(O)NR¹¹—C₁₋₆ alkyl-R¹², m) —C(O)—C₂₋₆ alkenyl-R¹², and n) —C(O)—C₂₋₆ alkynyl-R¹²;
alternatively, R⁴ and R⁵, taken together with the atoms to which they are bonded, form: 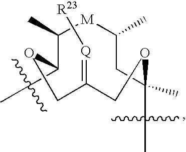
wherein
Q is CH or N, and
R²³ is —OR¹¹, or R¹¹;
R⁶ is selected from the group consisting of:
a) —OR¹¹, b) —C₁₋₆ alkoxy-R¹², c) —C(O)R¹¹, d) —OC(O)R¹¹, e) —OC(O)OR¹¹, f) —OC(O)NR¹¹R¹¹, and g) —NR¹¹R¹¹;
alternatively, R⁵ and R⁶ taken together with the atoms to which they are attached form a 5-membered ring by attachment to each other through a linker selected from the group consisting of:
a) —OC(R¹²)₂O—, b) —OC(O)O—, c) —OC(O)NR¹¹—, d) —NR¹¹C(O)O—, e) —OC(O)NOR¹¹—, f) —NOR¹¹—C(O)O—, g) —OC(O)NNR¹¹R¹¹—, h) —NNR¹¹R¹¹—C(O)O—, i) —OC(O)C(R¹²)₂—, j) —C(R¹²)₂C(O)O—, k) —OC(S)O—, l) —OC(S)NR¹¹—, m) —NR¹¹C(S)O—, n) —OC(S)NOR¹¹—, o) —NOR¹¹—C(S)O—, p) —OC(S)NNR¹¹R¹¹—, q) —NNR¹¹R¹¹—C(S)O—, r) —OC(S)C(R¹²)₂—, and s) —C(R¹²)₂C(S)O—;
alternatively, M, R⁵, and R⁶ taken together with the atoms to which they are attached form: 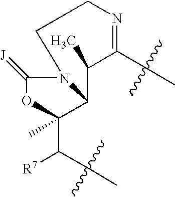
wherein J is selected from the group consisting of O and NR¹¹;
R6′ is selected from the group consisting of
a) —H, b) —C₁₋₄ alkyl, c) C₂₋₄ alkenyl, which can be further substituted with C₁₋₁₂ alkyl or one or more halogens, d) C₂₋₄ alkynyl, which can be further substituted with C-₁₋₁₂ alkyl or one or more halogens, e) aryl or heteroaryl, which can be further substituted with C-₁₋₁₂ alkyl or one or more halogens, f) —C(O)H, g) —COOH, h) —CN, i) —COOR¹¹, j) —C(O)NR¹¹R¹¹, k) —C(O)R¹¹, and l) —C(O)SR¹¹, wherein b) is further substituted with one or more substituents selected from the group consisting of aa) —OR¹¹, bb) halogen, cc) —SR¹¹, dd) C₁₋₁₂ alkyl, which can be further substituted with halogen, hydroxyl, C₁₋₆ alkoxy, or amino, ee) —OR¹¹, ff) —SR¹¹, gg) —NR¹¹R¹¹, hh) —CN, ii) —NO₂, jj) —NC(O)R¹¹, kk) —COOR¹¹, ll) —N₃, mm) ═N—O—R¹¹, nn) ═NR¹¹, oo) ═N—NR¹¹R¹¹, pp) ═N—NH—C(O)R¹¹, and qq) ═N—NH—C(O)NR¹¹R¹¹;
alternatively R6 and R6′ are taken together with the atom to which they are attached to form an epoxide, a carbonyl, an olefin, or a substituted olefin, or a C₃-C₇ carbocyclic, carbonate, or carbamate, wherein the nitrogen of said carbamate can be further substituted with a C₁-C₆ alkyl;
R⁷ is selected from the group consisting of:
a) C₁₋₆ alkyl, b) C₂₋₆ alkenyl, and c) C₂₋₆ alkynyl,
wherein any of a)—c) optionally is substituted with one or more R¹² groups;
R⁸ is selected from the group consisting of H and —C(O)R¹¹;
R⁹ is selected from the group consisting of H, OH, and OR¹¹;
R¹⁰ is selected from the group consisting of:
a) H, b) R¹¹, c) —C₁₋₆ alkyl-G-R¹², d) —C₂₋₆ alkenyl-G-R¹², and e) —C₂₋₆ alkynyl-G-R¹²,
wherein the C₁₋₆-alkyl, C₂₋₆ alkenyl, and C₂₋₆ alkynyl group in any of c)-e) optionally is substituted with one or more R¹² groups;
R¹¹, at each occurrence, independently is selected from the group consisting of:
a) H, b) C₁₋₆ alkyl, c) C₂₋₆ alkenyl, d) C₂₋₆ alkynyl, e) C₆₋₁₀ saturated, unsaturated, or aromatic carbocycle, f) 3-12 membered saturated, unsaturated, or aromatic heterocycle containing one or more heteroatoms selected from the group consisting of nitrogen, oxygen, and sulfur, g) —C(O)—C₁₋₆ alkyl, h) —C(O)—C₂₋₆ alkenyl, i) —C(O)—C₂₋₆ alkynyl, j) —C(O)—C₆₋₁₀ saturated, unsaturated, or aromatic carbocycle, k) —C(O)-3-12 membered saturated, unsaturated, or aromatic heterocycle containing one or more heteroatoms selected from the group consisting of nitrogen, oxygen, sulfur, l) —C(O)O—C₁₋₆ alkyl, m) —C(O)O—C₂₋₆ alkenyl, n) —C(O)O—C₂₋₆ alkynyl, o) —C(O)O—C₆₋₁₀ saturated, unsaturated, or aromatic carbocycle, p) —C(O)O-3-12 membered saturated, unsaturated, or aromatic heterocycle containing one or more heteroatoms selected from the group consisting of nitrogen, oxygen, and sulfur, and q) —C(O)NR¹³R¹³,
wherein any of b)—p) optionally is substituted with one or more R¹² groups,
alternatively, NR¹¹R¹¹ forms a 3-7 membered saturated, unsaturated or aromatic ring including the nitrogen atom to which the R¹¹ groups are bonded and optionally one or more moieties selected from the group consisting of: O, S(O)ₚ, and NR¹⁵;
R¹² is selected from the group consisting of:
a) R¹⁴, b) C₁₋₈ alkyl, c) C₂₋₈ alkenyl, d) C₂₋₈ alkynyl, e) C₃₋₁₂ saturated, unsaturated, or aromatic carbocycle, f) 3-12 membered saturated, unsaturated, or aromatic heterocycle containing one or more heteroatoms selected from the group consisting of nitrogen, oxygen, and sulfur, and g) —NR¹⁵C(O)OR¹⁵,
wherein any of b)-f) optionally is substituted with one or more R¹⁴ groups;
R¹³, at each occurrence, independently is selected from the group consisting of:
a) H, b) C₁₋₆ alkyl, c) C₂₋₆ alkenyl, d) C₂₋₆ alkynyl, e) C₃₋₁₀ saturated, unsaturated, or aromatic carbocycle, and f) 3-10 membered saturated, unsaturated, or aromatic heterocycle containing one or more heteroatoms selected from the group consisting of nitrogen, oxygen, and sulfur,
wherein any of b)-f) optionally is substituted with one or more moieties selected from the group consisting of:
carbonyl; formyl; F; Cl; Br; I; CN; NO₂; OR¹⁵; —S(O)ₚR¹⁵; —C(O)R¹⁵; —C(O)OR¹⁵; —OC(O)R¹⁵; —C(O)NR¹⁵R¹⁵; —OC(O)NR¹⁵R¹⁵; —C(═NR¹⁵R¹⁵; —C(R¹⁵)(R¹⁵)OR¹⁵; —C(R¹⁵)₂OC(O)R¹⁵; —C(R¹⁵)(OR¹⁵)(CH₂)ᵣNR¹⁵R¹⁵; —NR¹⁵R¹⁵; —NR¹⁵OR¹⁵; —NR¹⁵C(O)R¹⁵; —NR¹⁵C(O)OR¹⁵; —NR¹⁵C(O)NR¹⁵R¹⁵; —NR¹⁵S(O)ᵣR¹⁵; —C(OR¹⁵)(OR¹⁵R¹⁵; —C(R¹⁵)₂NR¹⁵R¹⁵; ═NR¹⁵; —C(S)NR¹⁵R¹⁵; —NR¹⁵C(S)R¹⁵; —OC(S)NR¹⁵R¹⁵; —NR¹⁵C(S)OR¹⁵; —NR¹⁵C(S)NR¹⁵R¹⁵; —SC(O)R¹⁵; C₁₋₈ alkyl, C₂₋₈ alkenyl; C₂₋₈ alkynyl; C₁₋₈ alkoxy; C₁₋₈ alkylthio; C₁₋₈ acyl; saturated, unsaturated, or aromatic C₃₋₁₀ carbocycle; and saturated, unsaturated, or aromatic 3-10 membered heterocycle containing one or more heteroatoms selected from the group consisting of nitrogen, oxygen, and sulfur,
alternatively, NR¹³R¹³ forms a 3-10 membered saturated, unsaturated or aromatic ring including the nitrogen atom to which the R¹³ groups are attached and optionally one or more moieties selected from the group consisting of O, S(O)ₚ, NR¹⁵, and N;
alternatively, CR¹³R¹³ forms a carbonyl group;
R¹⁴, at each occurrence, is selected from the group consisting of:
a) H, b) carbonyl, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣNR¹³(CR¹³R¹³)ₜR¹⁶, k) (CR¹³R¹³)ᵣOR¹⁶, l) (CR¹³R¹³)ᵣS(O)ₚ(CR¹³R¹³)ₜR¹⁶, m) (CR¹³R¹³)ᵣC(O)(CR¹³R¹³)ₜR¹⁶, n) (CR¹³R¹³)ᵣOC(O)(CR¹³R¹³)ₜR¹⁶, o) (CR¹³R¹³)ᵣSC(O)(CR¹³R¹³)ₜR¹⁶, p) (CR¹³R¹³ )ᵣC(O)O(CR¹³R¹³)ₜR¹⁶,q) (CR¹³R¹³ )ᵣNR¹³C(O)(CR¹³R¹³)ₜR¹⁶, r) (CR¹³R¹³)ᵣC(O)NR¹³(CR¹³R¹³)ₜR¹⁶, s) (CR¹³R¹³)ᵣC(═NR¹³)(CR¹³R¹³)ₜR¹⁶, t) (CR¹³R¹³)ᵣC(═NNR¹³R¹³)(CR¹³R¹³)ₜR¹⁶, u) (CR¹³R¹³)ᵣC(═NNR¹³C(O)R¹³)(CR¹³R¹³)ₜR¹⁶, v) (CR¹³R¹³)ᵣC(═NOR¹⁶)(CR¹³R¹³)ₜR¹⁶, w) (CR¹³R¹³)ᵣNR¹³C(O)O(CR¹³R¹³)ₜR¹⁶, x) (CR¹³R¹³)ᵣOC(O)NR¹³(CR¹³R¹³)ₜR¹⁶, y) (CR¹³R¹³)ᵣNR¹³C(O)NR¹³(CR¹³R¹³)ₜR¹⁶, z) (CR¹³R¹³)ᵣNR¹³S(O)ₚ(CR¹³R¹³)ₜR¹⁶, aa) (CR¹³R¹³)ᵣS(O)ₚNR¹³(CR¹³R¹³)ₜR¹⁶, bb) (CR¹³R¹³)ᵣNR¹³S(O)ₚNR¹³(CR¹³R¹³)ₜR¹⁶, cc) (CR¹³R¹³)ᵣNR¹³R¹³, dd) C₁₋₆ alkyl, ee) C₂₋₆ alkenyl, ff) C²⁻⁶ alkynyl, gg) (CR¹³R¹³)ᵣ—C₃₋₁₀ saturated, unsaturated, or aromatic carbocycle, and hh) (CR¹³R¹³)ᵣ—3-10 membered saturated, unsaturated, or aromatic heterocycle containing one or more heteroatoms selected from the group consisting of nitrogen, oxygen, and sulfur,
wherein any of dd)—hh) optionally is substituted with one or more R¹⁶ groups;
alternatively, two R¹⁴ groups may form —O(CH₂)ₛO—;
R¹⁵ is selected from the group consisting of:
a) H, b) C₁₋₆ alkyl, c) C₂₋₆ alkenyl, d) C₂₋₆ alkynyl, e) C₃₋₁₀ saturated, unsaturated, or aromatic carbocycle, f) 3-10 membered saturated, unsaturated, or aromatic heterocycle containing one or more heteroatoms selected from the group consisting of nitrogen, oxygen, and sulfur, g) —C(O)—C₁₋₆ alkyl, h) —C(O)—C₁₋₆ alkenyl, g) —C(O)—C₁₋₆ alkynyl, i) —C(O)—C₃₋₁₀ saturated, unsaturated, or aromatic carbocycle, and j) —C(O)—3-10 membered saturated, unsaturated, or aromatic heterocycle containing one or more heteroatoms selected from the group consisting of nitrogen, oxygen, and sulfur,
wherein any of b)-j) optionally is substituted with one or more moieties selected from the group consisting of H; F; Cl; Br; I; CN; NO₂; OH; NH₂; NH(C₁₋₆ alkyl); N(C₁₋₆ alkyl)₂; C₁₋₆ alkoxy; aryl; substituted aryl; heteroaryl; substituted heteroaryl; and C₁₋₆ alkyl, optionally substituted with one or more moieties selected from the group consisting of aryl, substituted aryl, heteroaryl, substituted heteroaryl, F, Cl, Br, I, CN, NO₂, and OH;
R¹⁶, at each occurrence, independently is selected from the group consisting of:
a) R¹⁷, b) C₁₋₆ alkyl, c) C₂₋₆ alkenyl, d) C₂₋₆ alkynyl, e) —C₃₋₁₀ saturated, unsaturated, or aromatic carbocycle, and f) —3-10 membered saturated, unsaturated, or aromatic heterocycle containing one or more heteroatoms selected from the group consisting of nitrogen, oxygen, and sulfur,
wherein any of b)-f) optionally is substituted with one or more R¹⁷ groups;
R¹⁷, at each occurrence, independently is selected from the group consisting of:
a) H, b) carbonyl, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣ(CR¹³R¹³), k) (CR¹³R¹³ ᵣOR¹¹, l) (CR¹³R¹³)ᵣS(O)ₚR¹³, m) (CR¹³R¹³)ᵣC(O)R¹³, n) (CR¹³R¹³)ᵣC(O)OR¹³, o) (CR¹³R¹³)ᵣOC(O)R¹³, p) (CR¹³R¹³)ᵣNR¹³C(O)R¹³, q) (CR¹³R¹³)ᵣC(O)NR¹³R¹³, r) (CR¹³R¹³)ᵣC(═NR¹³R¹³, s) (CR¹³R¹³)ᵣNR¹³C(O)NR¹³R¹³, t) (CR¹³R¹³)ᵣNR¹³S(O)ₚR¹³, u) (CR¹³R¹³)ᵣS(O)ₚNR¹³R¹³, v) (CR¹³R¹³)ᵣNR¹³S(O)ₚNR¹³R¹³, w ) C₁₋₆ alkyl, x) C₂₋₆ alkenyl, y) C₂₋₆ alkynyl, z) (CR¹³R¹³)ᵣ—C₃₋₁₀ saturated, unsaturated, or aromatic carbocycle, and aa) (CR¹³R¹³)ᵣ—3-10 membered saturated, unsaturated, or aromatic heterocycle containing one or more heteroatoms selected from the group consisting of nitrogen, oxygen, and sulfur,
wherein any of w)-aa) optionally is substituted with- one or more moieties selected from the group consisting of R¹³; F; Cl; Br; I; CN; NO₂; —OR¹³; —NH₂; —NH(C₁₋₆ alkyl); —N(C₁₋₆ alkyl)₂; C₁₋₆ alkoxy; C₁₋₆ alkylthio; and C₁₋₆ acyl;
R¹⁸, at each occurrence, independently is selected from the group consisting of:
a) H, b) OR¹⁵, c) —O—C₁₋₆ alkyl-OC(O)R¹⁵, d) —O—C₁₋₆ alkyl-OC(O)OR¹⁵, e) —O—C₁₋₆ alkyl-OC(O)NR¹⁵R¹⁵, f) —O—C₁₋₆ alkyl-C(O)NR¹⁵R¹⁵, g) —O—C₁₋₆ alkyl-NR¹⁵C(O)R¹⁵, h) —O—C₁₋₆ alkyl-NR¹⁵C(O)OR¹⁵, i) —O—C₁₋₆ alkyl-NR¹⁵C(O)NR¹⁵R¹⁵, j) —O—C₁₋₆ alkyl-NR¹⁵C(═NH)NR¹⁵R¹⁵, k) —O—C₁₋₆ alkyl-S(O)ₚR¹⁵, l) —O—C₂₋₆alkenyl-OC(O)R¹⁵, m) —O—C₂₋₆ alkenyl-OC(O)OR¹⁵, n) —O—C₂₋₆ alkenyl-OC(O)NR¹⁵R¹⁵, o) —O—C₂₋₆ alkenyl-C(O)NR¹⁵R¹⁵, p) —O—C₂₋₆ alkenyl-NR¹⁵C(O)R¹⁵, q) —O—C₂₋₆ alkenyl-NR¹⁵C(O)OR¹⁵, r) —O—C₂₋₆ alkenyl-NR¹⁵C(O)NR¹⁵R¹⁵, s) —O—C₂₋₆ alkenyl-NR¹⁵C(═NH)NR¹⁵R¹⁵, t) —O—C₂₋₆ alkenyl-S(O)ₚR¹⁵, u) —O—C₂₋₆ alkynyl-OC(O)R¹⁵, v) —O—C₂₋₆ alkynyl-OC(O)OR¹⁵, w) —O—C₂₋₆ alkynyl-OC(O)NR¹⁵R¹⁵, x) —O—C₂₋₆ alkynyl-C(O)NR¹⁵R¹⁵, y) —O—C₂₋₆ alkynyl-NR¹⁵C(O)R¹⁵, z) —O—C₂₋₆ alkynyl-NR⁵C(O)OR¹⁵, aa) —O—C₂₋₆ alkynyl-NR¹⁵C(O)NR¹⁵R¹⁵, bb) —O—C₂₋₆ alkynyl-NR¹⁵C(═NH)NR¹⁵R¹⁵, cc) —O—C₂₋₆ alkynyl-S(O)ₚR¹⁵; and dd) —NR¹⁵R¹⁵;
alternatively, two R¹⁸ groups taken together form ═O, ═NOR¹⁵, or ═NNR¹⁵R¹⁵;
R¹⁹ is R¹²;
R²⁰ is selected from the group consisting of:
a) R¹³), b) F, c) Cl, d) Br, e) I, f) CN, g) NO₂, and h) —OR¹¹;
alternatively, R¹⁹ and R²⁰ taken together are —O(CH₂)ᵣO—;
R²¹, at each occurrence, independently is selected from the group consisting of:
a) H, b) F, c) Cl, d) Br, e) I, f) CN, g) —OR¹¹, h) NO₂, i) —NR¹¹R¹¹, j) C₁₋₆ alkyl, k) C₁₋₆ acyl, and l) C₁₋₆ alkoxy;
R²² is selected from the group consisting of:
a) C₁₋₆ alkyl, b) C₂₋₆ alkenyl, c) C₂₋₆ alkynyl, d) C₁₋₆ acyl, e) C₁₋₆ alkoxy, f) C₁₋₆ alkylthio, g) saturated, unsaturated, or aromatic C₅₋₁₀ carbocycle, h) saturated, unsaturated, or aromatic 5-10 membered heterocycle containing one or more heteroatoms selected from the group consisting of nitrogen, oxygen, and sulfur, i) —O—C₁₋₆ alkyl-saturated, unsaturated, or aromatic 5-10 membered heterocycle containing one or more heteroatoms selected from the group consisting of nitrogen, oxygen, and sulfur, j) —NR¹¹—C₁₋₆ alkyl-saturated, unsaturated, or aromatic 5-10 membered heterocycle containing one or more heteroatoms selected from the group consisting of nitrogen, oxygen, and sulfur, k) saturated, unsaturated, or aromatic 10-membered bicyclic ring system optionally containing one or more heteroatoms selected from the group consisting of nitrogen, oxygen, and sulfur, 1) saturated, unsaturated, or aromatic 13-membered tricyclic ring system optionally containing one or more heteroatoms selected from the group consisting of nitrogen, oxygen, and sulfur, m) —OR¹¹, n) —NR¹¹R¹¹, o) S(O)ᵣR¹¹, and p) R²¹,
wherein any of a)-l) optionally is substituted with one or more R¹² groups;
alternatively, R²² and one R²¹ group, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;
R²³ at each occurrence, independently is selected from the group consisting of:
a) hydrogen; b) an electron-withdrawing group; c) aryl; d) substituted aryl;
e) heteroaryl; f) substituted heteroaryl; and g) C₁₋₆ alkyl, optionally substituted with one or more R¹² groups;
alternatively, any R²³ and any R²⁰, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;
p, at each occurrence, is selected from the group consisting of 0, 1, and 2;
r, at each occurrence, is selected from the group consisting of 0, 1, and 2;
s, at each occurrence, is selected from the group consisting of 1, 2, 3, or 4;
t, at each occurrence, is selected from the group consisting of 0, 1, or 2;
u, at each occurrence, is selected from the group consisting of 1, 2, 3, 4, or 5; and,
v, at each occurrence, is selected from the group consisting of 0, 1, 2, or 3.

2. A compound according to claim 1, having the formula selected from the group consisting of: 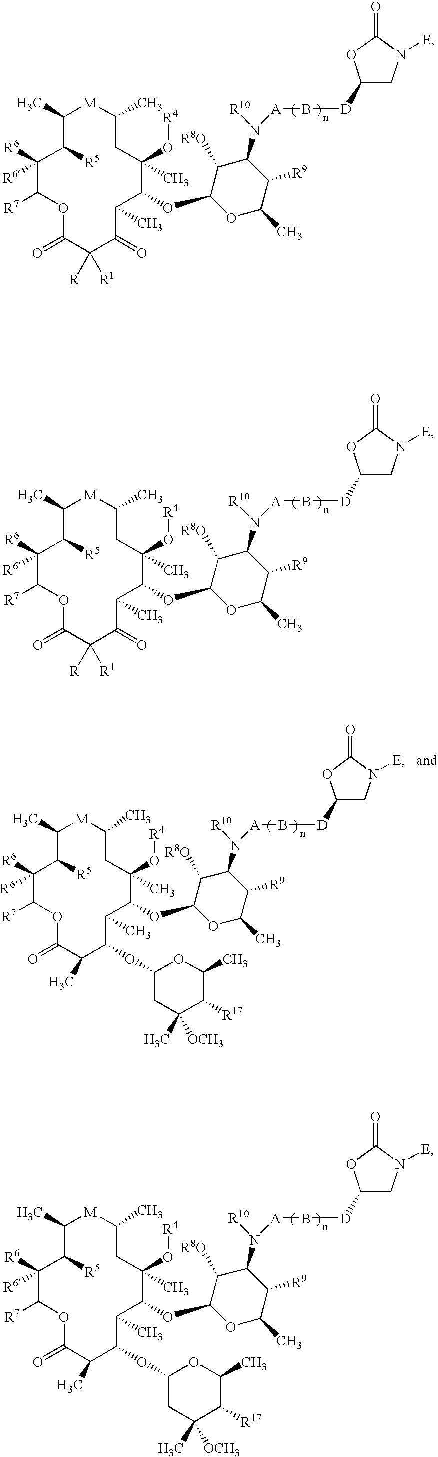
or pharmaceutically acceptable salt, ester, or prodrug thereof,
wherein A, B, n, D, E, R, R¹, R⁴, R⁵, R⁶, R⁶′, R⁷, R⁸, R⁹, R¹⁰ and R¹⁷ are as defined in claim 1.

3. A compound according to claim 1, having the formula selected from the group consisting of: 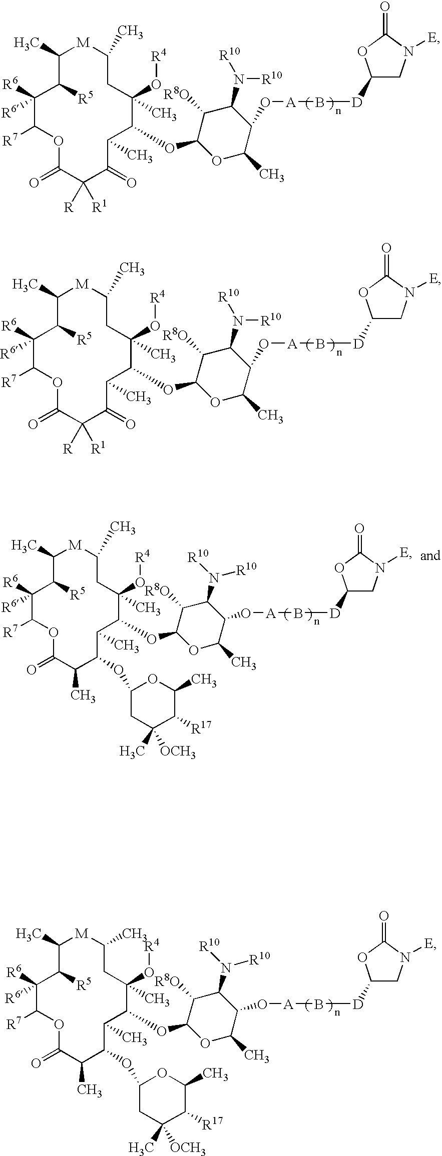
or pharmaceutically acceptable salt, ester, or prodrug thereof,
wherein A, B, n, D, E, R, R¹, R⁴, R⁵, R⁶, R⁶′, R⁷, R⁸, R⁹, R10 and R¹⁷ are as defined in claim 1.

4. A compound according to claim 1, having the formula selected from the group consisting of: 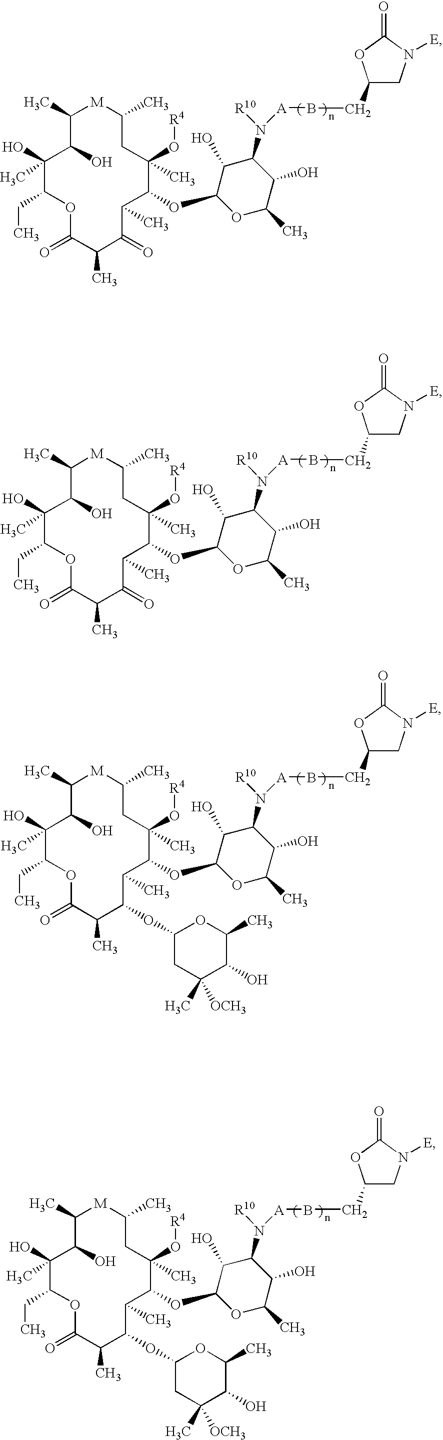 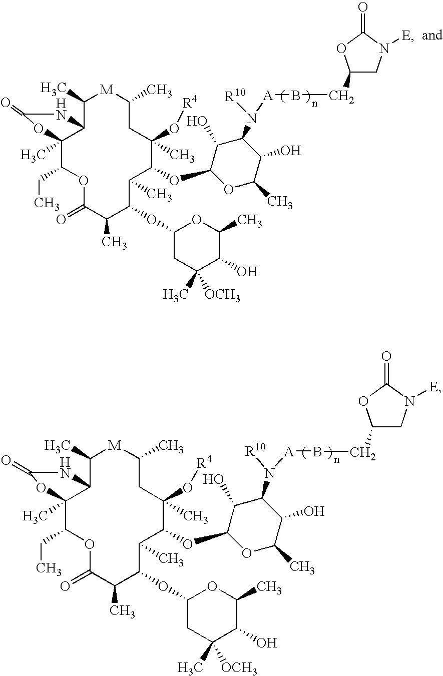
or a pharmaceutically acceptable salt, ester, or prodrug thereof,
wherein A, B, n, E, R⁴, and R¹⁰ are as defined in claim 1.

5. A compound according to claim 1, having the formula selected from the group consisting of: 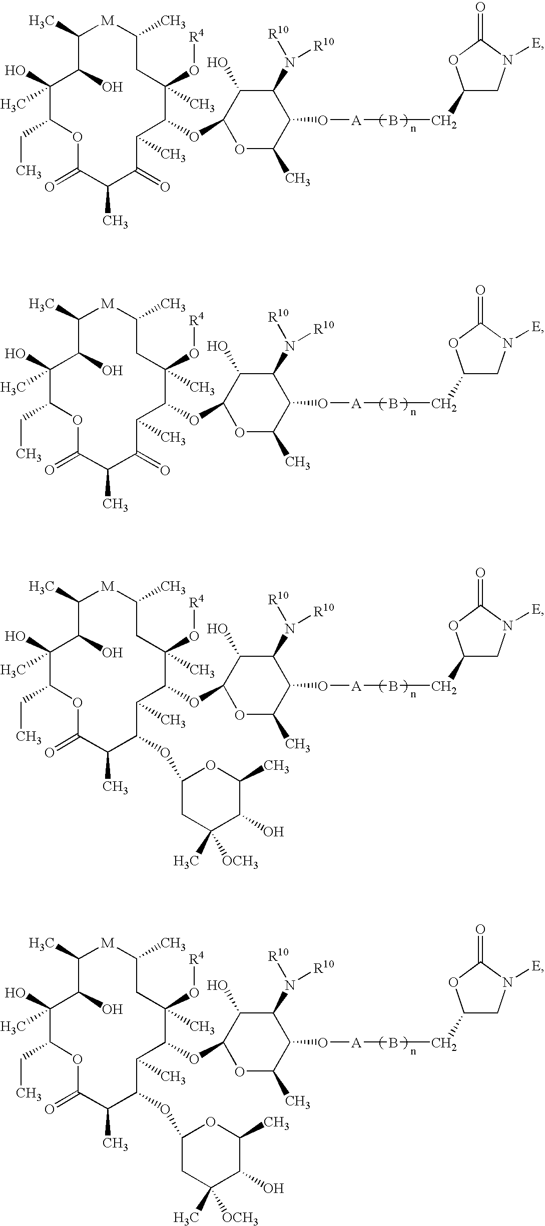 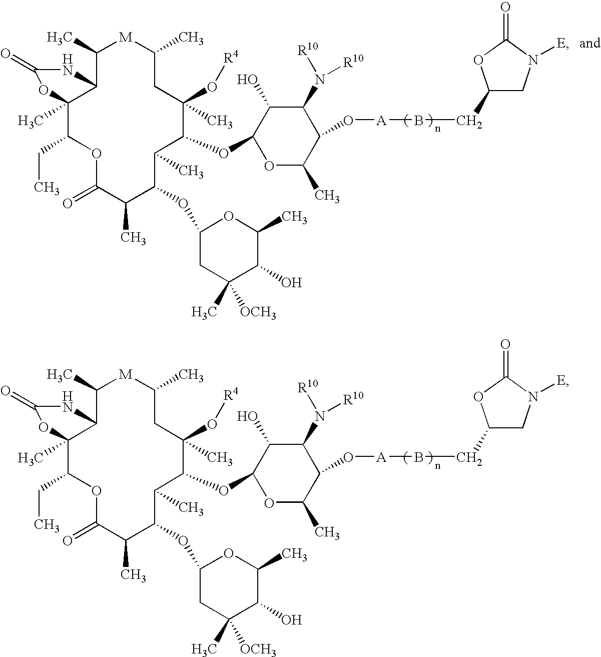
or a pharmaceutically acceptable salt, ester, or prodrug thereof,
wherein A, B, n, B, R⁴, and R¹⁰ are as defined in claim 1.

6. A compound according to claim 1, having the formula selected from the group consisting of: 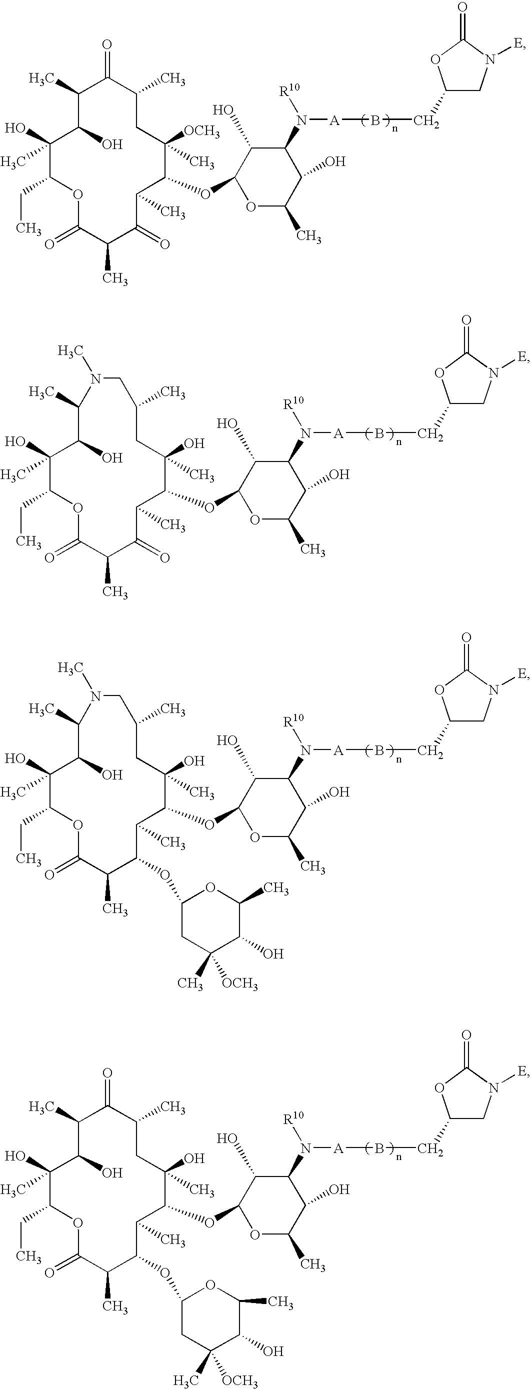 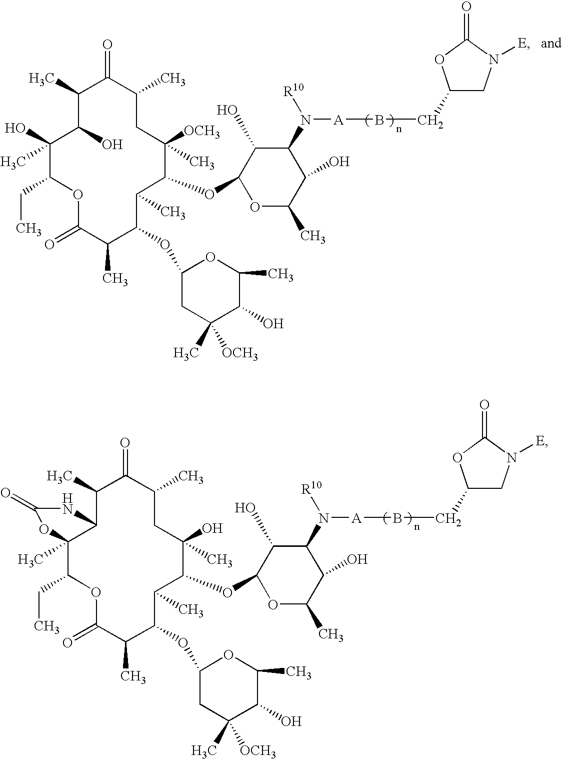
or a pharmaceutically acceptable salt, ester, or prodrug thereof,
wherein A, B, n, B, and R¹⁰ are as defined in claim 1.

7. A compound according to claim 1, having the formula selected from the group consisting of: 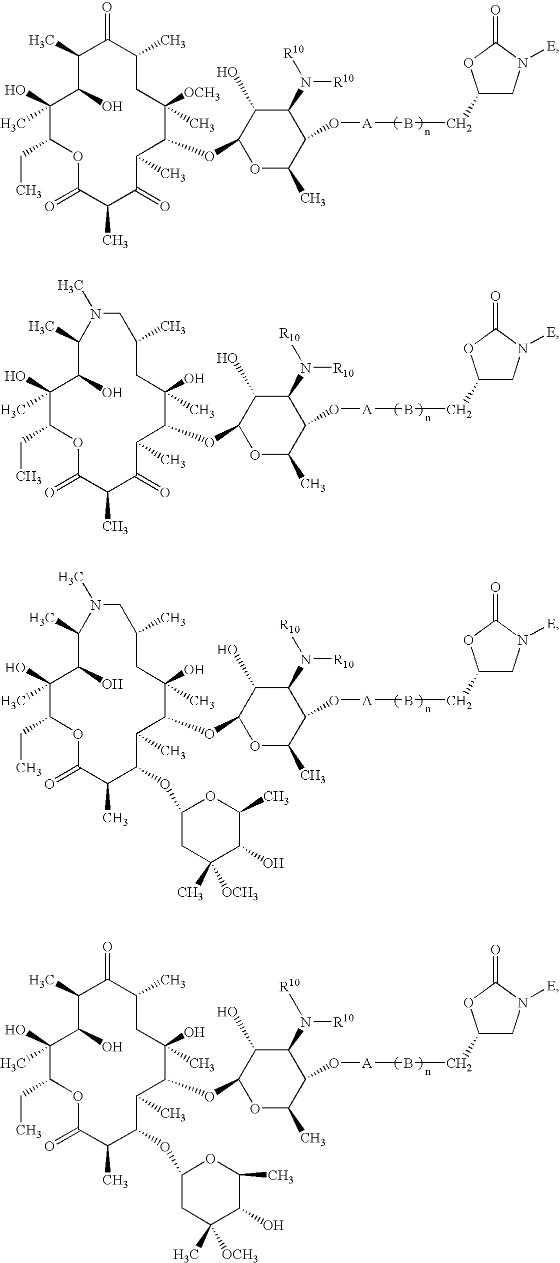 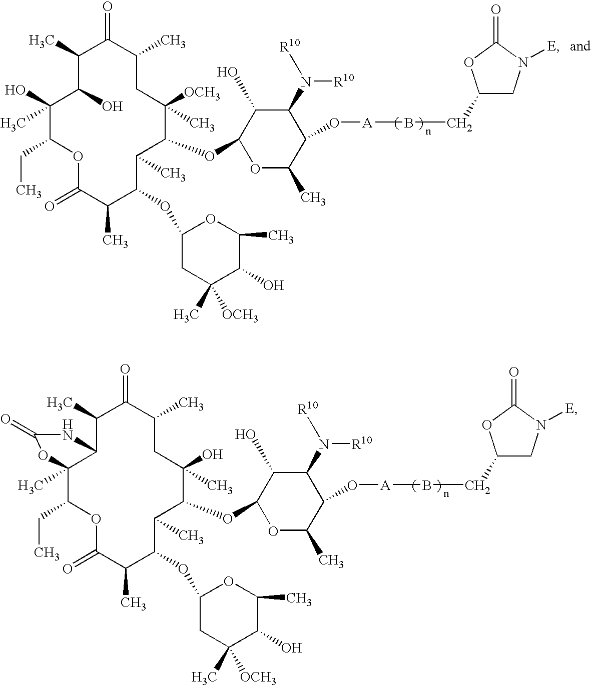
or a pharmaceutically acceptable salt, ester, or prodrug thereof,
wherein A, B, n, E, and R¹⁰ are as defined in claim 1.

8. The compound according to claim 1, wherein n is 1.

9. The compound according to claim 1, wherein A—(B)ₙ—D is:
A—C(O)NH—D.

10. The compound according to claim 1, wherein A—(B)ₙ—D is:
A—SO₂NH—D.

11. The compound according to claim 1, wherein A—(B)ₙ—D is:
A—C(S)NH—D.

12. A compound having the formula
M—(CH₂)ₘ—B—O
or a pharmaceutically acceptable salt, ester, or prodrug thereof,
wherein M is a macrolide selected from the group consisting of 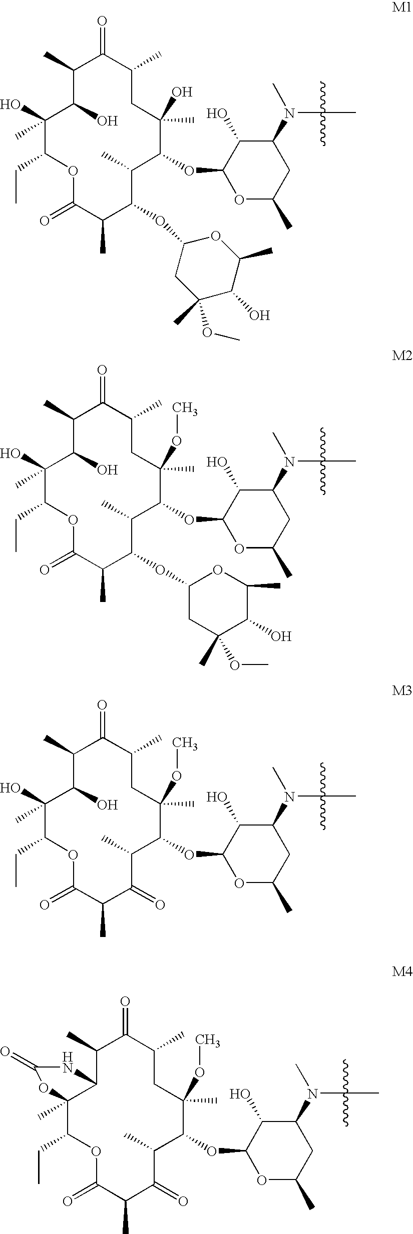 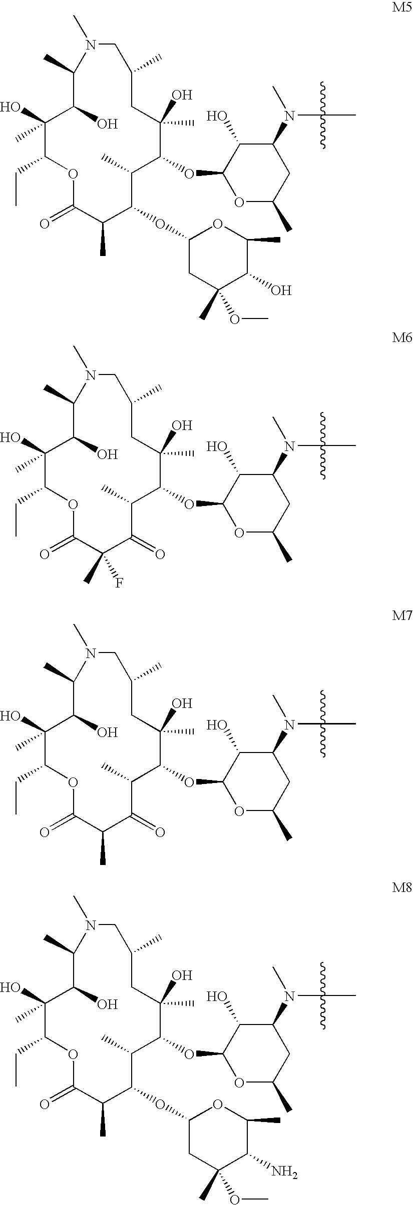 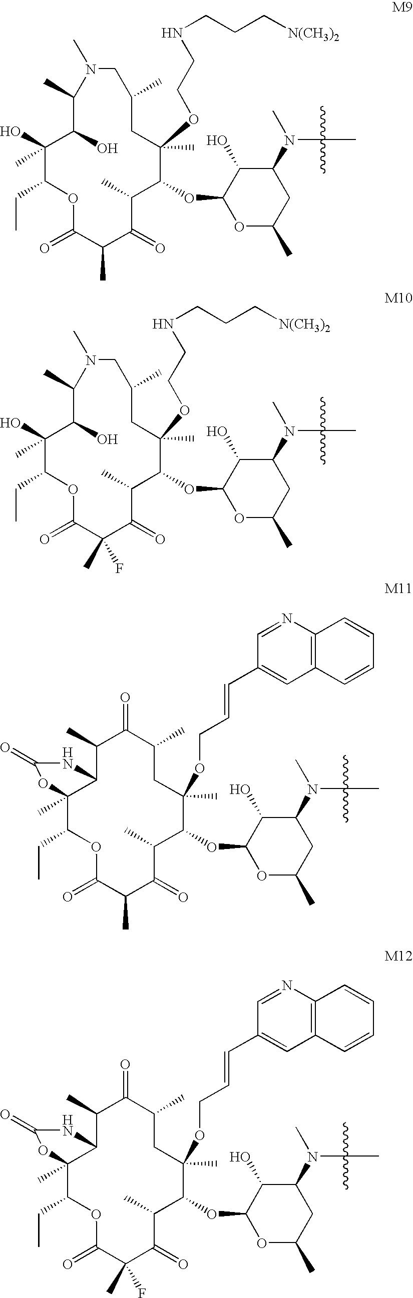 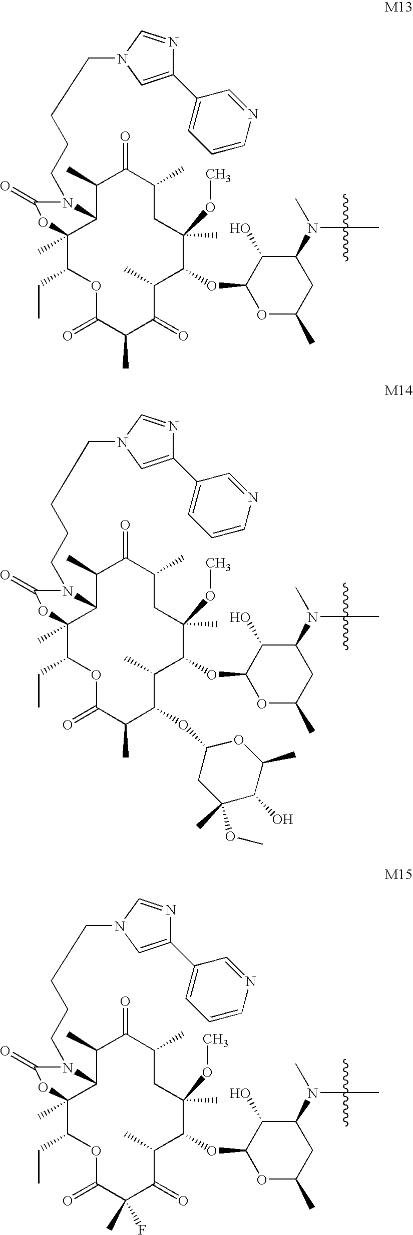 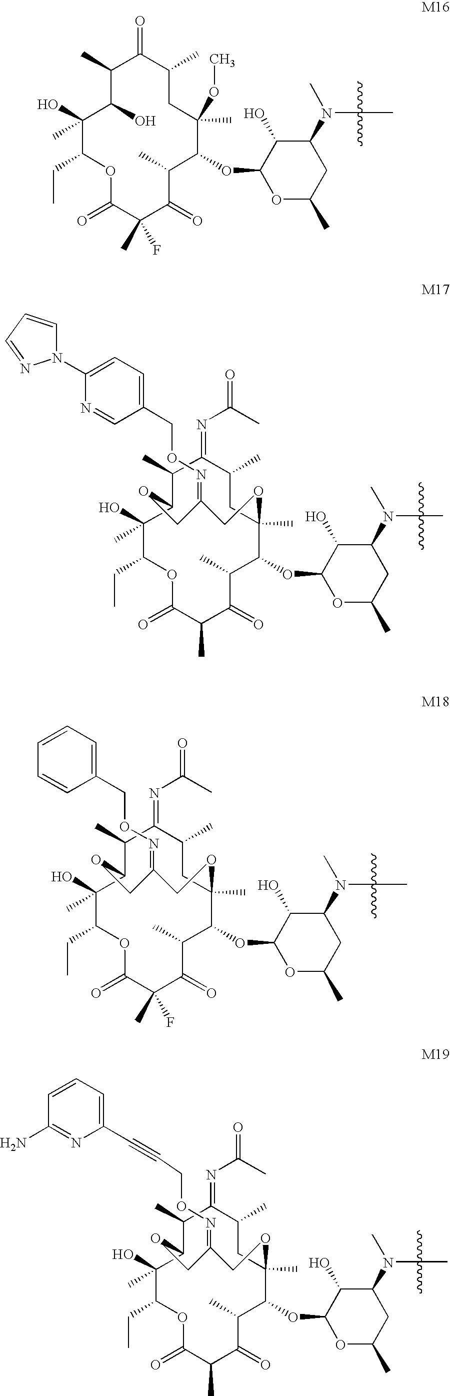 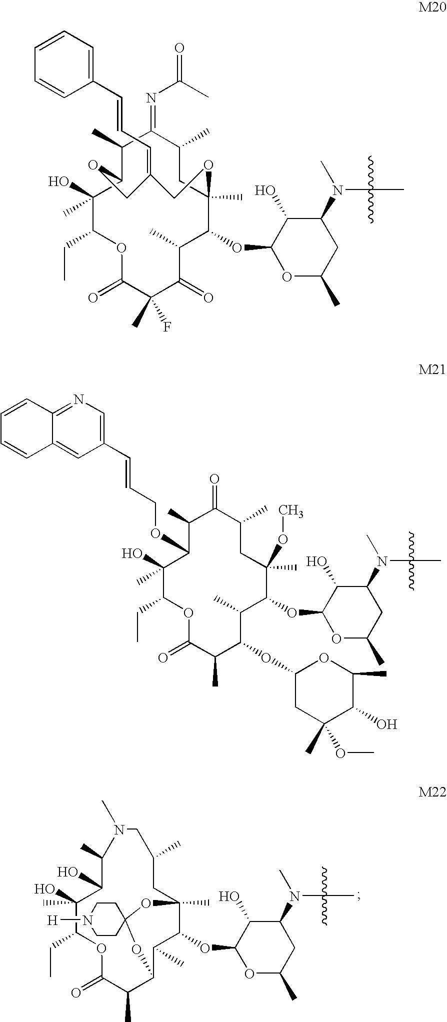
B is a linker selected from the group consisting of 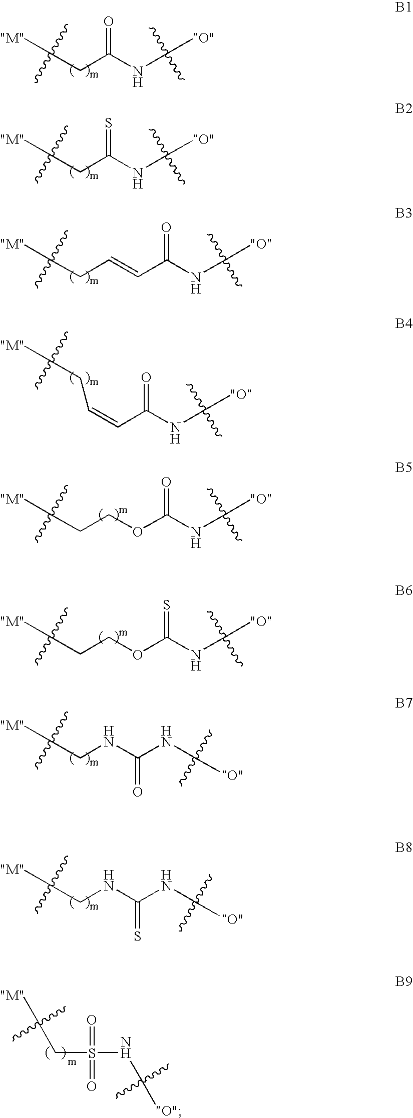
O is a heterocyclic side chain selected from the group consisting of 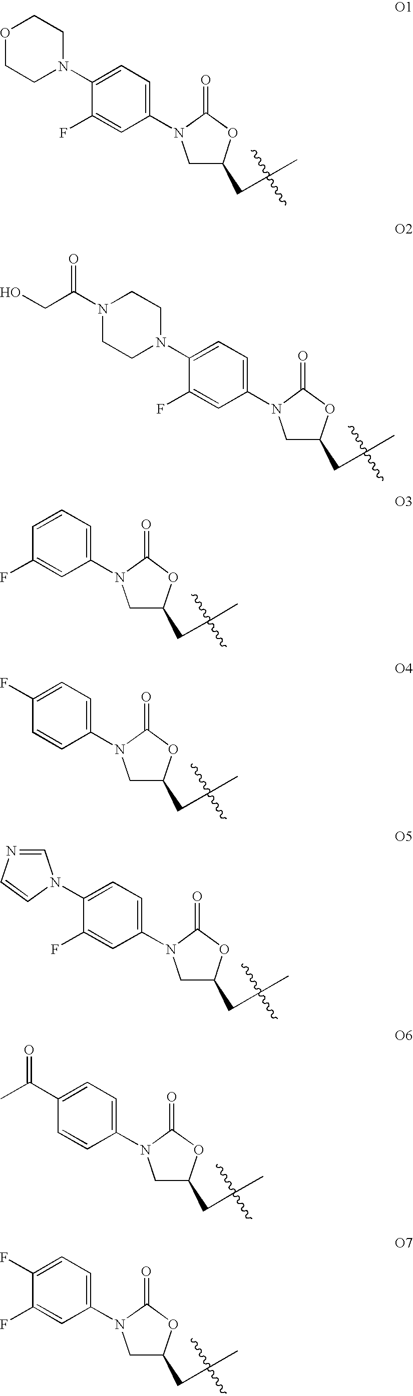 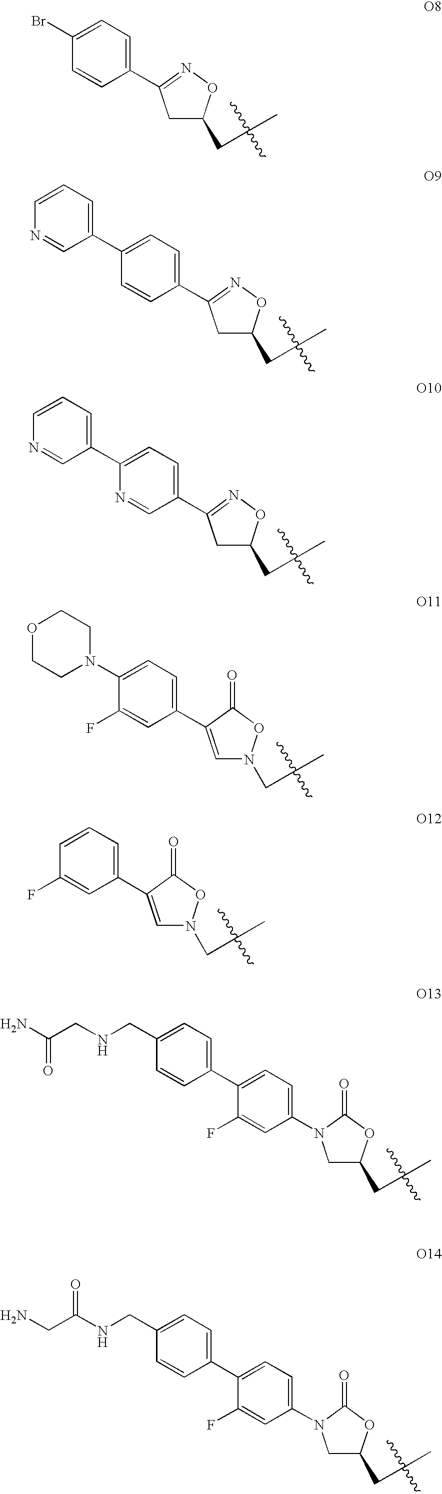 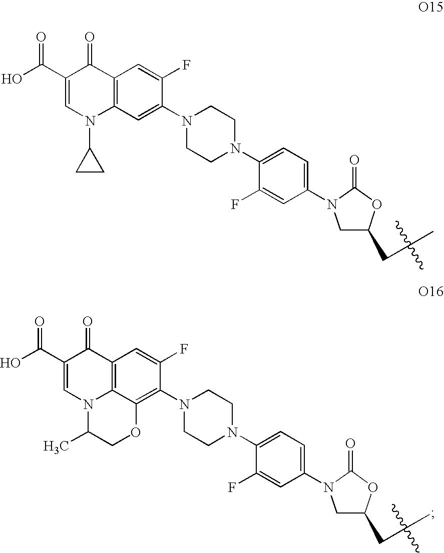
and m is an integer from 1-4.

13. A compound having the formula selected from the group consisting of: 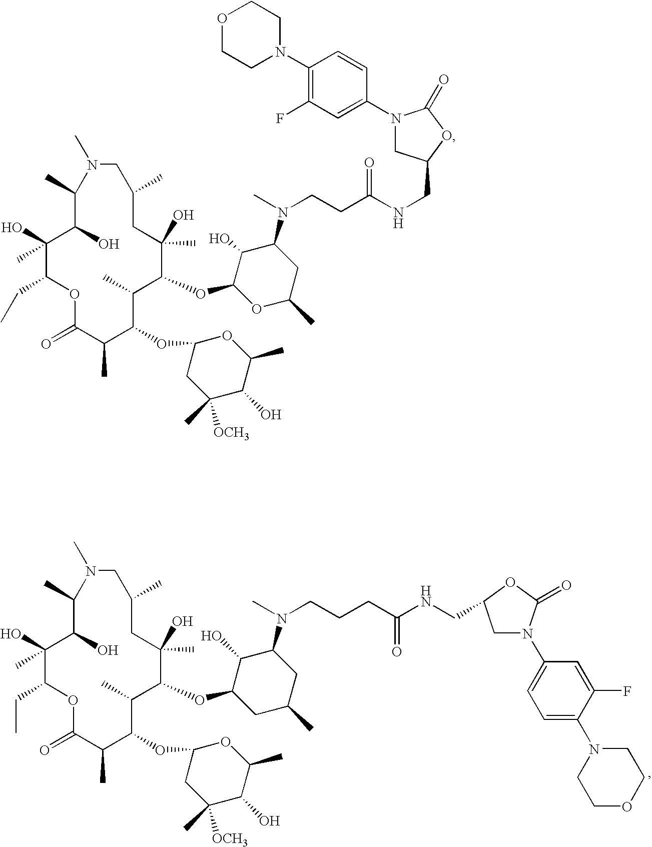 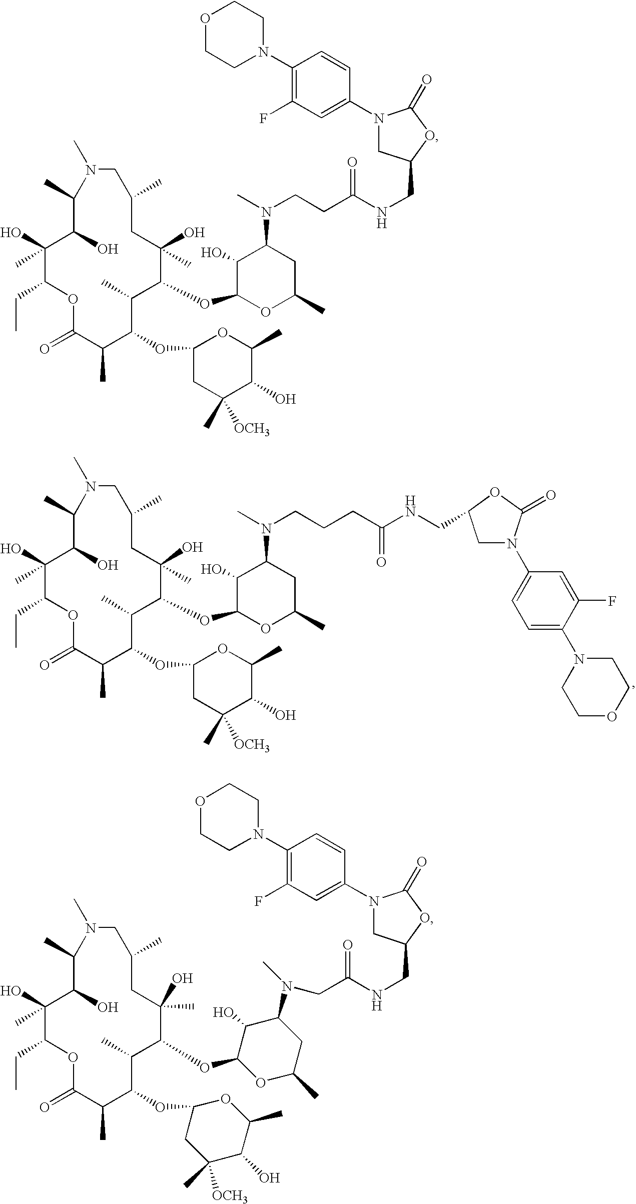 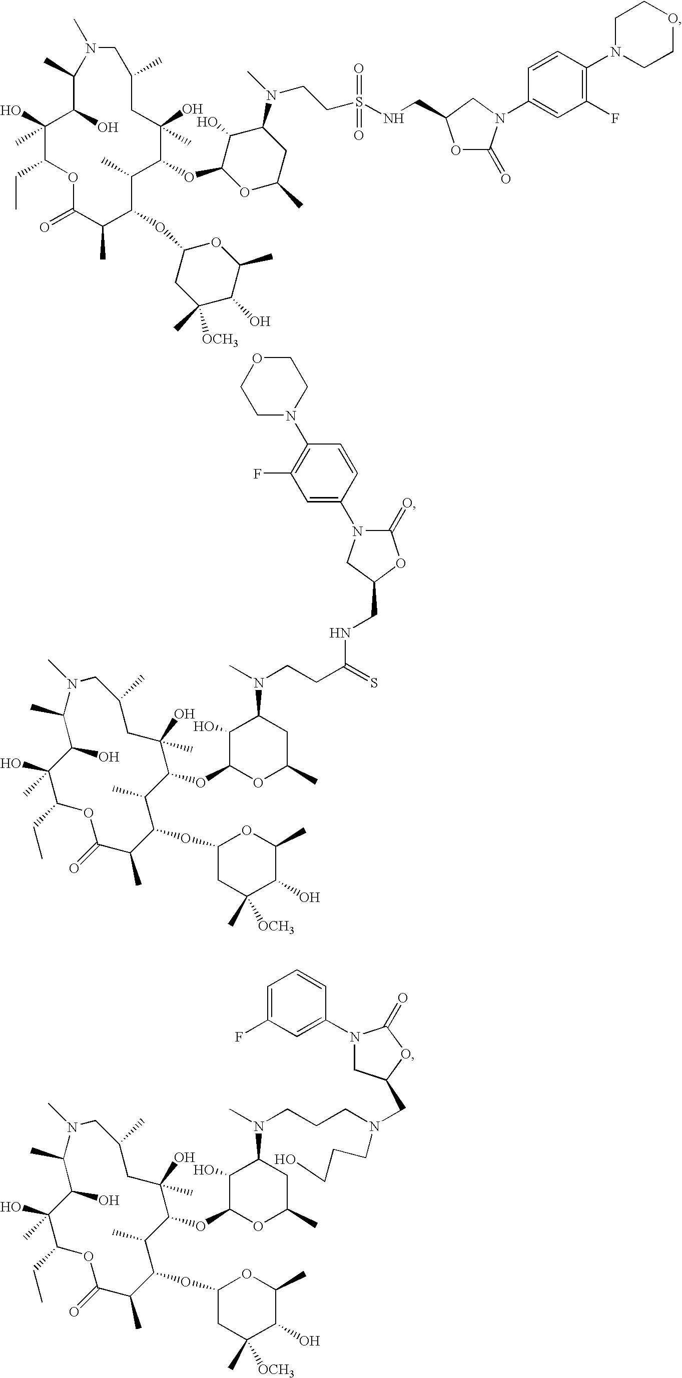 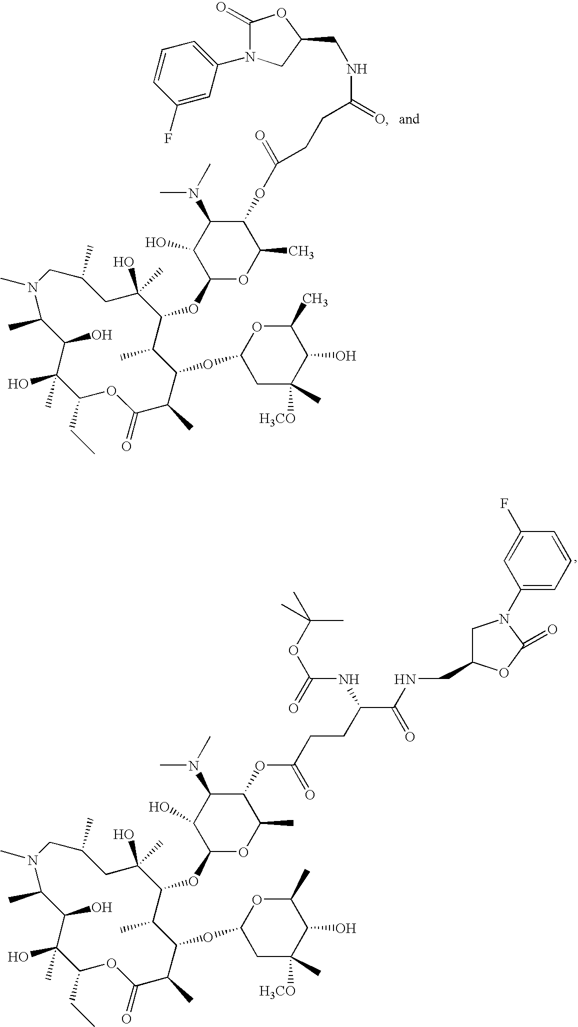
or a pharmaceutically acceptable salt, ester, or prodrug thereof.

14. A pharmaceutical composition comprising a compound according to claim 1 and a pharmaceutically acceptable carrier.

15. A method of treating a microbial infection in a mammal comprising administering to the mammal an effective amount of a compound according to claim 1.

16. A method of treating a fungal infection in a mammal comprising administering to the mammal an effective amount of a compound according to claim 1.

17. A method of treating a parasitic disease in a mammal comprising administering to the mammal an effective amount of a compound according to claim 1.

18. A method of treating a proliferative disease in a mammal comprising administering to the mammal an effective amount of a compound according to claim 1.

19. A method of treating a viral infection in a mammal comprising administering to the mammal an effective amount of a compound according to claim 1.

20. A method of treating an inflammatory disease in a mammal comprising administering to the mammal an effective amount of a compound according to claim 1.

21. A method of treating a gastrointestinal motility disorder in a mammal comprising administering to the mammal an effective amount of a compound according to claim 1.

22. The method according to claim 15 wherein the compound is administered orally, parentally, or topically.

23. A method of synthesizing a compound according to claim 1.

24. A medical device containing a compound according to claim 1.

25. The medical device according to claim 24, wherein the device is a stent.
